(19) United States
(12) Patent Application Publication
(10) Pub. No.: US 2025/0049957 A1
(43) Pub. Date: Feb. 13, 2025

(54) CSF TRANSPORT PATHWAY FOR DELIVERY OF AGENTS TO INNER EAR

(71) Applicants: University of Rochester, Rochester, NY (US); University of Copenhagen, Copenhagen (DK); Karolinska Institutet, Solna (SE)

(72) Inventors: Maiken Nedergaard, Rochester, NY (US); Barbara K. Mathiesen, Copenhagen (DK); Christopher R. Cederroth, Stockholm (SE); Barbara Canlon, Stockholm (SE)

(73) Assignees: University of Rochester, Rochester, NY (US); University of Copenhagen, Copenhagen (DK); Karolinska Institutet, Solna (SE)

(21) Appl. No.: 18/722,690

(22) PCT Filed: Dec. 22, 2022
(86) PCT No.: PCT/US2022/082225

§ 371 Date: Jun. 21, 2024

Related U.S. Application Data
(60) Provisional application No. 63/293,209, filed on Dec. 23, 2021.

(51) Int. Cl. A61K 48/00; A61P 27/16; C07K 14/705; C12N 15/86

(52) U.S. Cl. CPC A61K 48/0058; A61P 27/16; C07K 14/70571; C12N 15/86; C12N 2750/14143

(57) ABSTRACT

This disclosure relates to delivery of various agents to the inner ear of a subject.

### CROSS REFERENCE TO RELATED APPLICATION

This application claims priority to U.S. Provisional Application No. 63/293,209 filed on Dec. 23, 2021. The content of the application is incorporated herein by reference in its entirety.

### FIELD OF THE INVENTION

This disclosure relates to delivery of various agents to the inner ear of a subject.

### BACKGROUND

Hearing loss affects about 12% of individuals over the age of twelve, or around 30 million Americans (NIDCD 2010). The likelihood of having bilateral hearing loss doubles each decade after the age of fifty, to the point that over 60% of people aged 70 or older have hearing loss. An estimated 2.45 billion of individuals are predicted to have mild-to-complete hearing impairment by 2050. Current ameliorating therapy using hearing aids or cochlear implants remain insufficient in restoring the full capacities of auditory function. Gene therapy administered through intracochlear or intravestibular injections in early post-natal mice have recently been shown to restore hearing in models of genetic deafness (Bankoti et al., 2021). However, gene therapy beyond the post-natal stages has proven unsuccessful (Bankoti et al., 2021) and these procedures are invasive surgeries that can cause damage to inner ear structures. There is a need for therapeutic and methods for restoring hearing loss.

### SUMMARY

This disclosure addresses the need mentioned above in a number of aspects.

In one aspect, the disclosure provides a method for delivering an agent to an inner ear of a subject comprising administering the agent to the cerebrospinal fluid (CSF) in the subject.

The agent can be a therapeutic agent. Examples of the therapeutic agent include one or more selected from the group consisting of a small molecule, a large molecule, a peptide, a protein, an antibody, a nucleic acid, a vector, and a cell. In one embodiment, the therapeutic agent is the nucleic acid or the vector. The nucleic acid or the vector can comprise a gene that encodes a protein or polypeptide. Examples of the gene, protein or polypeptide include VGLUT3, MYO7A (USH1B), USH1C, CDH23, PCDH15 (USH1F), SANS (USH1G), USH2A, ADGRV1/VLGR1, WHRN (DFNB31), USH3A (CLRN1), HARS, Cx26 (GJB2), Cx30 (GJB6), Cx29 (GJC3), Cx31 (GJB3), ACTG1, FSCN2, RDX, POU4F3, TRIOBP, TPRN, XIRP2, ATOH1, GFI1, CHRNA9, CIB3, CDH23, PCDH15, KNCN, DFNB59, OTOF, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, CABP2, EPS8, EPS8L2, ESPN, ESPNL, PRPH2, STRC, SLC8A2, ZCCHC12, LRTOMT2, LRTOMT1, USH1C, ELFN1, TTC24, DYTN, KCP, CCER2, LRTM2, KCNA10, NTF3, CLRN1, CLRN2, SKOR1, TCTEX1 D1, FCRLB, SLC17A8, GRXCR2, BDNF, SERPINE3, NHLH1, HSP70, HSP90, ATF6, PERK, IRE1, BIP, GJB2, and USH1G. In one embodiment, the gene encodes one selected from the group consisting of SEQ ID NOs: 1-15. In one embodiment, the gene is VGLUT3. In one embodiment, the gene encodes SEQ ID NO: 1.

In another embodiment, the therapeutic agent is selected from the group consisting of a Jun N terminal kinase inhibitor, a brain derived neurotrophic factor ligand, a PPAR agonist, a gamma-secretase inhibitor, a beta-catenin stimulator, a stem cell stimulator, an activator of Lgr5-positive epithelial stem cell proliferation, a cell differentiation modulator, a sensory hair cell regenerating compound, a glutathione peroxidase stimulator, a Vitamin K dependent protein C stimulator, an otoprotectant, a chemoprotectant, a hair cell regenerating compound, a Glycogen synthase kinase-3 inhibitor, a 5-HT 3 receptor antagonist, a calcineurin inhibitor, a free radical scavenger, an anti-inflammatory agent, an apoptosis inhibitor, a neuroprotectant, a cyclin-dependent kinase-2 inhibitor, an apoptotic protease-activating factor 1 inhibitor, a metabotropic glutamate receptor 7 antagonist, a toll-like receptor (TLR) antagonist, a TLR-2 antagonist, a TLR-4 antagonist, a TLR-9 antagonist, a tropomyosin receptor kinase (Trk)-C agonist, a Heat shock protein stimulator, a Guanylate cyclase stimulator, a PDE 5 inhibitor, an antiviral agent, a DNA polymerase inhibitor, a Transferase inhibitor, a KCNC potassium channel 1 modulator, aKCNC potassium channel 2 modulator, and a HSF1 gene stimulator. For example, the therapeutic agent can be selected from the group consisting of brimapitide, OTO-413, pioglitazone, OTO-510, NXT-596, FX-322, pioglitazone hydrochloride, PIPE-505, otopotin, LY-3056480, ebselen, SPI-3005, ancrod, sodium thiosulfate, ACOU-085, OTO-6XX, DB-020, ORC-13661, FX-345, arazasetron besylate, disufenton sodium, acetylcysteine, AC-102, AZD-5438, LPT-99, NT-12, OR-112, PGT-117, P-13, PIPE-336, OR-102C, ebselen, small heat shock protein, TOP-M119, AP-001, ganciclovir, dendrogenin B, AUT-00206, Dexamethasone (DEX), DEX-salvianolic acid B (DEX-SAL) conjugate, HB-097, and plexaris.

In a further embodiment, the therapeutic agent is a cell, such as a stem cell. Examples of the stem cell include an embryonic stem cell, ES-like stem cell, fetal stem cell, adult stem cell, pluripotent stem cell, induced pluripotent stem cell, multipotent stem cell, oligopotent stem cell, or unipotent stem cell.

The above-mentioned vector can be a plasmid, cosmid, artificial chromosome, or viral vector. The viral vector can be any suitable viral vector, including but not limited to an adeno-associated virus (AAV) vector, an adenovirus vector, and a lentivirus vector. In a preferred embodiment, the viral vector is an AAV vector. The serotype of the AAV vector can be one selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PUP.S.

The agent mentioned above can be an imaging agent. Examples of the imaging agent include one or more selected from the group consisting of an organic molecule, a fluorophore, a metal ion, a salt or chelate, a particle, a peptide, a protein, a nucleic acid, a polymer and a liposome.

In some embodiments, the method described above can further comprise examining a level of the agent in the inner ear after the administering step.

In a second aspect, the disclosure provides a method of treating a subject having or at risk of developing hearing loss. The method comprises administering an effective amount of a therapeutic agent to the CSF in the subject. The therapeutic agent can be one selected from the group consisting of a small molecule, a large molecule, a peptide, a protein, an antibody, a nucleic acid, a vector, and a cell.

In some embodiments, the therapeutic agent is the nucleic acid or the vector. The nucleic acid or the vector can comprise a gene that encodes a protein or polypeptide. Examples of the gene include VGLUT3, MYO7A (USH1B), USH1C, CDH23, PCDH15 (USH1F), SANS (USH1G), USH2A, ADGRV1/VLGR1, WHRN (DFNB31), USH3A (CLRN1), HARS, Cx26 (GJB2), Cx30 (GJB6), Cx29 (GJC3), Cx31 (GJB3), ACTG1, FSCN2, RDX, POU4F3, TRIOBP, TPRN, XIRP2, ATOH1, GFI1, CHRNA9, CIB3, CDH23, PCDH15, KNCN, DFNB59, OTOF, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, CABP2, EPS8, EPS8L2, ESPN, ESPNL, PRPH2, STRC, SLC8A2, ZCCHC12, LRTOMT2, LRTOMT1, USH1C, ELFN1, TTC24, DYTN, KCP, CCER2, LRTM2, KCNA10, NTF3, CLRN1, CLRN2, SKOR1, TCTEX1 D1, FCRLB, SLC17A8, GRXCR2, BDNF, SERPINE3, NHLH1, HSP70, HSP90, ATF6, PERK, IRE1, BIP, GJB2, and USH1G. In one embodiment, the gene encodes one selected from the group consisting of SEQ ID NOs. 1-15. In one embodiment, the gene is VGLUT3. In one embodiment, the gene encodes SEQ ID NO: 1.

The agent can be (i) the nucleic acid that is a RNAi agent or an antisense sequence comprising a sequence that is complementary to a segment of a target gene, or (ii) the vector that encodes the RNAi agent or the antisense sequence. Examples may include siRNA based therapeutics such as HES gene modulators developed by Otologic Pharmaceutics Inc.

In some embodiments, the therapeutic agent is selected from the group consisting of a Jun N terminal kinase inhibitor, a brain derived neurotrophic factor ligand, a PPAR agonist, a gamma-secretase inhibitor, a beta-catenin stimulator, a stem cell stimulator, an activator of Lgr5-positive epithelial stem cell proliferation, a cell differentiation modulator, a sensory hair cell regenerating compound, a glutathione peroxidase stimulator, a Vitamin K dependent protein C stimulator, an otoprotectant, a chemoprotectant, a hair cell regenerating compound, a Glycogen synthase kinase-3 inhibitor, a 5-HT 3 receptor antagonist, a calcineurin inhibitor, a free radical scavenger, an anti-inflammatory agent, an apoptosis inhibitor, a neuroprotectant, a cyclin-dependent kinase-2 inhibitor, an apoptotic protease-activating factor 1 inhibitor, a metabotropic glutamate receptor 7 antagonist, a toll-like receptor (TLR) antagonist, a TLR-2 antagonist, a TLR-4 antagonist, a TLR-9 antagonist, a tropomyosin receptor kinase (Trk)-C agonist, a Heat shock protein stimulator, a Guanylate cyclase stimulator, a PDE 5 inhibitor, an antiviral agent, a DNA polymerase inhibitor, a Transferase inhibitor, a KCNC potassium channel 1 modulator, aKCNC potassium channel 2 modulator, and a HSF1 gene stimulator. For example, the therapeutic agent can be selected from the group consisting of brimapitide, OTO-413, pioglitazone, OTO-510, NXT-596, FX-322, pioglitazone hydrochloride, PIPE-505, otopotin, LY-3056480, ebselen, SPI-3005, ancrod, sodium thiosulfate, ACOU-085, OTO-6XX, DB-020, ORC-13661, FX-345, arazasetron besylate, disufenton sodium, acetylcysteine, AC-102, AZD-5438, LPT-99, NT-12, OR-112, PGT-117, P-13, PIPE-336, OR-102C, ebselen, small heat shock protein, TOP-M119, AP-001, ganciclovir, dendrogenin B, AUT-00206, Dexamethasone (DEX), DEX-salvianolic acid B (DEX-SAL) conjugate, HB-097, and plexaris.

In some embodiments, the therapeutic agent is a cell, such as a stem cell. Examples of the stem cell include an embryonic stem cell, ES-like stem cell, fetal stem cell, adult stem cell, pluripotent stem cell, induced pluripotent stem cell, multipotent stem cell, oligopotent stem cell, or unipotent stem cell.

The above-mentioned vector can be a plasmid, cosmid, artificial chromosome, or viral vector. The viral vector can be any suitable viral vector, including but not limited to an adeno-associated virus (AAV) vector, an adenovirus vector, and a lentivirus vector. In a preferred embodiment, the viral vector is an AAV vector. The serotype of the AAV vector can be one selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PUP.S.

The hearing loss mentioned above can be genetic hearing loss, such as autosomal dominant hearing loss, autosomal recessive hearing loss, or X-linked hearing loss. The hearing loss can be acquired hearing loss, such as noise-induced hearing loss, age-related hearing loss, disease or infection-related hearing loss, head trauma-related hearing loss, or ototoxic drug-induced hearing loss.

In a third aspect, the disclosure features a method of increasing expression of a gene in an inner ear cell of a subject, The method includes administering to the CSF in the subject a nucleic acid or a vector comprising the gene. Examples of the inner ear cell include inner hair cells, outer hair cells, vestibular hair cells, cochlear cells and vestibular supporting cells. The vector can be a plasmid, cosmid, artificial chromosome, or viral vector (e.g., an AAV vector, an adenovirus vector, and a lentivirus vector). In a preferred embodiment, the viral vector is an AAV vector. The serotype of the AAV vector can be one selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. The subject may have or be at risk of developing hearing loss. The hearing loss can be genetic hearing loss (e.g., autosomal dominant hearing loss, autosomal recessive hearing loss, or X-linked hearing loss) or acquired hearing loss (e.g., noise-induced hearing loss, age-related hearing loss, disease or infection-related hearing loss, head trauma-related hearing loss, or ototoxic drug-induced hearing loss). In one embodiment, the method may further comprise evaluating the hearing of the subject prior to the administering step, after the administering step, or at both time points.

In the methods described above, the agent, therapeutic agent, nucleic acid, or vector is administered intracisternally or intrathecally.

The details of one or more embodiments of the disclosure are set forth in the description below. Other features, objectives, and advantages of the disclosure will be apparent from the description and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, 1G, 1H and 1I are a set of graphs showing that cerebrospinal fluid tracers readily enter cochlea via cochlear aqueduct. FIG. 1A shows the experimental setup for MRI and CT scans. FIG. 1B show the anatomy of the ear. FIG. 1C shows dynamic contrast-enhanced magnetic resonance (DCE-MR) imaging after cisterna magna injection of Gadovist (0.6 kDa) and shows that the tracer enter the brain cisterns and the cochlea (white arrow). FIGS. 1D and 1E are time-series showing that Gadovist in the subarachnoid space enters the inner ear through an opening identified as the cochlear aqueduct (red arrow in D). FIG. 1F shows time series of MRI regions of interest (ROI) means (+/−SEM) and depicts that Gadovist arrive outside the cochlear aqueduct, then move into the cochlear aqueduct and lastly arrive in the ear (arrival time statistics in FIG. 4A). FIGS. 1G, 1H, and 1I show high spatial resolution CT time series of ROI means (+/−SEM) performed after cisterna magna injection of Omnipaque (0.8 kDa), confirming that tracer signal first increases in the aqueduct (red arrows in G) before it moves into the base of scala tympani, and that no significant tracer arrives in scala vestibuli within the 30 minutes of scanning. Repeated t-test against zero mean with Sidak's multiple comparison test; * P<0.0021.

FIGS. 2A, 2B, 2C, 2D, 2E, and 2F are a set of graphs showing cochlear aqueduct is wrapped by a lymphatic-like membrane. FIG. 2A is a schematic of cochlear. FIG. 2B and FIG. 2C show that microspheres (0.2 μm) injected in cisterna magna are present in the cochlear aqueduct and are often associated with fiber-like structures located therein. The microspheres are also present in the base of scala tympani and in the cochlea. FIG. 2D shows lymphatic marker analysis and reveals that an arachnoid membrane forms a diaphragm-like structure (green arrowheads) at the cochlear end of the aqueduct (red arrowheads), which is PROX1, CRABP2 and podoplanin positive but LYVE1 negative. There is Iba1 and LYVE1 positive macrophage-like cells attached to the diaphragm (red arrowheads). FIG. 2E shows a large number of cisterna magna injected microspheres get entrapped in the membrane and is phagocytized by the Iba1 positive microspheres.

FIGS. 3A, 3B, 3C, 3D, 3E, 3F, 3G, 3H and 3I are a set of graphs showing that CSF-delivered VGLUT3 overexpressing virus rescues hearing and the auditory afferent synapse in a mouse model of human deafness. FIGS. 3A and 3B show auditory brainstem responses (FIG. 3A) and distortion products of otoacoustic emissions (FIG. 3B) of Slc17a8+/+ (green) and −/− (red) mice at baseline (dashed line, open circles) and 2 weeks post-injection (full line, filled circles) treated with either aCSF or VGLUT3-AAV (rescued). Since injection of GFP-AAV had no impact on hearing, aCSF and GFP-AAV were merged together and served as controls. Data are mean+/−SEM, and individual responses are shown in the background. WT treated with VGLUT3 ABR thresholds were elevated at 32 and 40 kHz by 25 and 16 dB, respectively when compared to baseline (two-way ANOVA, Treatment Factor: F_{(1,96)}=7.98, p=0.0058; Frequency Factor: F_{(5,96)}=88.4, p<0.0001, (A), whereas other frequencies remained unaffected. KO mice displayed improved ABR thresholds at all frequencies except 48 kHz (two-way ANOVA, Genotype Factor: F_{(1,72)}=237, p<0.0001; Frequency Factor: F_{(5,72)}=24.9, p<0.0001). Hearing in the KO mice was at a level similar to WT animals, with the exception of 24 kHz (two-way ANOVA, Genotype Factor: F_{(1,72)}=18, p<0.0001; Frequency Factor: F_{(5,72)}=72, p<0.0001; 24 kHz, p=0.0065 in Sidak's post-hoc testing), where KO mice had higher thresholds by 5 dB than WT mice. The function of the outer hair cells as measured by distortion product emissions (B) was not affected by the aCSF or viral treatment in neither the WT nor the KO mice. FIG. 3C shows averaged auditory brainstem response wave forms with SEM (shaded) for Slc17a8+/+(green) and −/− (red) mice showing the evoked responses with decreasing stimulus intensity. FIGS. 3D, 3E, and 3F show representative micrographs of Slc17a8+/+(D) and −/− (E) mice treated with aCSF or −/− rescued (F) stained for CtBP2 (red), GluR2 (green) and Myosin VIIa (blue), or VGLUT3 (white) at basal, middle and apical turns. Rows of inner hair cells (IHC) are marked. Bar is 5 m. FIGS. 3G, 3H, and 3I show quantification of pre-synaptic ribbons (G), post-synaptic GluR2 (H) and paired synapses (I) from Slc17a8+/+(green) and −/− (red) mice treated with aCSF (empty bars) or rescued (filled bars). Data are mean+/−SEM, with individual values shown, N=3-10. Stars in indicate significant differences; *: p<0.05; **: p<0.01; ***: p<0.0001; three-way ANOVA with Sidak's post hoc analysis.

FIGS. 4A, 4B, 4C, 4D, 4E, 4F, and 4G are a set of graphs showing that Aqp4-independent but dispersive mechanisms drive CSF transport into inner ear. FIG. 4A is MRI data showing tracer arriving significantly faster in the cochlear aqueduct compared to the ear. FIG. 4B shows time activity curves for all ROIs measured in the CT. No notable rise in cochlear nerve. FIGS. 4C, 4D, and 4E show that there was no significant difference in tracer influx after CM injection in wild type and AQP4-knockouts. FIG. 4F shows tracer concentrations in the cochlear aqueduct, shown on both a time- and a spatial axis from CT images (dots) correspond to dispersion-model predictions (full lines). FIG. 4G shows the dispersion coefficients estimated from data in cochlear aqueduct similar to the Stokes-Einstein free diffusion of Omnipaque in water.

FIG. 5 shows that microspheres are abundant in the cochlear aqueduct after 1 h and have reached the basal part of the ear after 24 hours.

FIGS. 6A, 6B, and 6C shows CSF-delivered virus result in significantly higher expression in the hair cells compared to intravenous injected viruses. FIG. 6A shows a schematic representation of the virus entering the ear from the CSF. FIG. 6B shows expression of virus in the inner ear after cisterna magna injection and intravenous injection. The virus is highly expressed in the cochlea base and middle in the cisterna magna injected animals. FIG. 6C shows quantification of virus expression in inner ear in cisterna magna injected animals compared to intravenous injected animals show significantly higher expression in the base and middle of the cisterna magna injected animals.

FIGS. 7A and 7B show auditory brainstem response wave 1 amplitude (A) and latency (B) from Slc17a8+/+ (green) and −/− (red) treated with aCSF (dashed lines, open circles) or rescued (full lines, filled circles) showed that the KO rescued group recovered to 62% of the WT treated group (two-way ANOVA, Genotype Factor: F_{(3,94)}=24, p<0.0001; Stimulus intensity Factor: F_{(4,94)}=18.8, p<0.0001. Wave 1, 3 and 5 peak latencies in Slc17a8+/+ (green) and −/− (red) mice treated with aCSF (empty bars) or rescued (filled bars). Data are mean+/−SEM, with individual values shown, N=6-10. Null symbol represents the lack of ABR response for the Slc17a8 −/− untreated mice. Stars indicate significant differences; *: p<0.05; **: p<0.01; one-way ANOVA with Sidak's post hoc analysis.

FIG. 8 shows placement of regions of interest in CT.

### DETAILED DESCRIPTION

This disclosure relates to delivery various agents to an inner ear of a subject, which can be used to restore hearing loss. Certain aspects of this disclosure are based, at least in part, on unexpected discoveries that hearing is rescued in adult deaf mice by using a cerebrospinal fluid route to deliver therapeutics, such as gene therapeutics, to the inner ear.

### Cerebrospinal Fluid as Deliver Route to Inner Ear.

Cerebrospinal fluid (CSF) flow via the glymphatic system is emerging as a new approach for brain-wide delivery of drug delivery. The inner ear fluids and CSF are directly connected via the cochlear aqueduct, which exhibits lymphatic-like characteristics. Real-time magnetic resonance imaging, computed tomography and optical fluorescence microscopy showed that large CSF tracers reach the inner ear by dispersive transport via the cochlear aqueduct in adult mice.

As disclosed in the examples herein, a single intracisternal injection of adeno-associated virus expressing VGLUT3 transduced inner hair cells, rescued hearing thresholds, and re-established auditory synapses in Slc17A8 −/− mice, a model of autosomal dominant deafness. CSF transport thus comprises a novel pathway for effective gene delivery to the adult inner ear.

An estimated 2.45 billion of individuals are predicted to have mild-to-complete hearing impairment by 2050 (Collaborators, 2021). Current ameliorating therapy using hearing aids or cochlear implants remain insufficient in restoring the full capacities of auditory function. Gene therapy administered through intracochlear or intravestibular injections in early post-natal mice have recently been shown to restore hearing in models of genetic deafness (Bankoti et al., 2021). However, gene therapy beyond the post-natal stages has proven unsuccessful (Bankoti et al., 2021) and these procedures are invasive surgeries that can cause damage to inner ear structures.

In the brain, cerebrospinal fluid (CSF) reaches deep brain regions by transport along the perivascular spaces in what has been dubbed the glymphatic system. Glymphatic fluid transport plays an important homeostatic role as fluid efflux clears metabolic waste products, such as amyloid-β, tau and lactate (Iliff et al., 2012; Xie et al., 2013). The hair cells in the inner ear are protected by the blood-labyrinth barrier but are also highly metabolically active (Nyberg et al., 2019; Spinelli et al., 2012). Yet, the ear is similar to the brain and eye largely devoid of lymphatic vessels (Salt et al., 2015). Recently an ocular glymphatic clearance system has been described (Wang et al., 2020), raising the possibility that the ear, like other neural tissue, exports metabolic waste products by active CSF transport. From a therapeutic perspective, it is important to note that the glymphatic fluid transport bypasses the blood brain barrier (BBB) and can be used as a route for delivering BBB-impermeable drugs to the brain. Therapeutic agents in CSF can reach deep brain structures within minutes by perivascular CSF transport (Lilius et al., 2019; Plog et al., 2018).

To inventors' knowledge no previous studies have demonstrated the possibility of using the CSF route for delivering gene therapy to rescue hearing in a mouse model of human deafness. Herein, inventors assessed the connection between inner ear fluid and CSF surrounding the brain and whether the connection can be used for drug delivery. It was found that CSF within minutes enters the inner ear and this easily accessible CSF route can deliver various agents, including viral gene therapy, to restore the function of an inherited mutation associated with deafness in an adult mouse model of human deafness. Accordingly, the CSF route can be used to deliver various agents to the inner ear.

### Therapeutic Agents

As disclosed herein, the agent can be a therapeutic agent. Examples of the therapeutic agent include a small molecule, a large molecule, a peptide, a protein, an antibody, a nucleic acid, a vector, or a cell.

In one embodiment, the therapeutic agent is selected from the group consisting of Jun N terminal kinase inhibitor, brain derived neurotrophic factor ligand, PPAR agonist, gamma-secretase inhibitor, beta-catenin stimulator, stem cell stimulator, activator of Lgr5-positive epithelial stem cell proliferation, cell differentiation modulator, sensory hair cell regenerating compound, glutathione peroxidase stimulator, Vitamin K dependent protein C stimulator, otoprotectant, chemoprotectant, hair cell regenerating compound, Glycogen synthase kinase-3 inhibitor, 5-HT 3 receptor antagonist, calcineurin inhibitor, free radical scavenger, anti-inflammatory agent, apoptosis inhibitor, neuroprotectant, cyclin-dependent kinase-2 inhibitor, apoptotic protease-activating factor 1 inhibitor, metabotropic glutamate receptor 7 antagonist, toll-like receptor (TLR) antagonist, TLR-2 antagonist; TLR-4 antagonist, TLR-9 antagonist, tropomyosin receptor kinase (Trk)-C agonist, Heat shock protein stimulator, Guanylate cyclase stimulator; PDE 5 inhibitor, antiviral agent, DNA polymerase inhibitor; Transferase inhibitor, KCNC potassium channel 1 modulator; KCNC potassium channel 2 modulator, and HSF1 gene stimulator. For example, the therapeutic agent can be selected from the group consisting of brimapitide, OTO-413, pioglitazone, OTO-510, NXT-596, FX-322, pioglitazone hydrochloride, PIPE-505, otopotin, LY-3056480, ebselen, SPI-3005, ancrod, sodium thiosulfate, ACOU-085, OTO-6XX, DB-020, ORC-13661, FX-345, arazasetron besylate, disufenton sodium, acetylcysteine, AC-102, AZD-5438, LPT-99, NT-12, OR-112, PGT-117, P-13, PIPE-336, OR-102C, ebselen, small heat shock protein, TOP-M119, AP-001, ganciclovir, dendrogenin B, AUT-00206, Dexamethasone (DEX), DEX-salvianolic acid B (DEX-SAL) conjugate, HB-097, and plexaris.

In another embodiment, the therapeutic agent is a compound that promotes proliferation and/or differentiation of an inner ear cell, such as a hair cell or a supporting cell. Examples of such a compound include, but are not limited to, a retinoid receptor signaling activator, a Wnt signaling activator, a bone morphogenetic protein (BMP) signaling inhibitor, a cyclin-dependent kinase (CDK) activator, an E box-dependent transcriptional activator; a Notch signaling activator, a histone deacetylase (HDAC) inhibitor, a protein degradation inhibitor, a PI3K-Akt signaling inhibitor, and a cAMP response element binding protein (CREB) activator. Additional examples are described in US 20190010449, the disclosure of which is incorporated herein by reference.

In one example, the therapeutic agent comprises a fusosome. A “fusosome” refers to a bilayer of amphipathic lipids enclosing a lumen or cavity and a fusogen that interacts with the amphipathic lipid bilayer. In some embodiments, the fusosome comprises a nucleic acid. In some embodiments, the fusosome is a membrane enclosed preparation. In some embodiments, the fusosome is derived from a source cell. Fusosomes can take various forms. For example, in some embodiments, a fusosome described herein is derived from a source cell. A fusosome may be or comprise, e.g., an extracellular vesicle, a microvesicle, a nanovesicle, an exosome, a microparticle, or any combination thereof. In some embodiments, a fusosome is released naturally from a source cell, and in some embodiments, the source cell is treated to enhance formation of fusosomes. In some embodiments, the fusosome is between about 10-10,000 nm in diameter, e.g., about 30-100 nm in diameter. In some embodiments, the fusosome comprises one or more synthetic lipids.

In some embodiments, the fusosome is or comprises a virus, e.g., a retrovirus, e.g., a lentivirus. For instance, in some embodiments, the fusosome's bilayer of amphipathic lipids is or comprises the viral envelope. The viral envelope may comprise a fusogen, e.g., a fusogen that is endogenous to the virus or a pseudotyped fusogen. In some embodiments, the fusosome's lumen or cavity comprises a viral nucleic acid, e.g., a retroviral nucleic acid, e.g., a lentiviral nucleic acid. The viral nucleic acid may be a viral genome. In some embodiments, the fusosome further comprises one or more viral non-structural proteins, e.g., in its cavity or lumen.

Fusosomes may have various structures or properties that facilitate delivery of a payload to a target cell. For instance, in some embodiments, the fusosome and the source cell together comprise nucleic acid(s) sufficient to make a particle that can fuse with a target cell. In embodiments, these nucleic acid(s) encode proteins having one or more of (e.g., all of) the following activities: gag polyprotein activity, polymerase activity, integrase activity, protease activity, and fusogen activity.

### Nucleic Acids and Vectors

As disclosed herein, a gene therapy may be delivered via a recombinant expression cassette or expression vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Examples of vectors include plasmids, cosmids, viruses (bacteriophage, animal viruses, and plant viruses), and artificial chromosomes (e.g., YACs). One of skill in the art would be well equipped to construct a vector through standard recombinant techniques.

The term “expression vector” refers to a vector containing a nucleic acid sequence coding for at least part of a gene product capable of being transcribed. In some cases, RNA molecules are then translated into a protein, polypeptide, or peptide. In other cases, such as with miRNAs, these sequences are not translated. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### Promoter and Transgene

A promoter is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression.

It may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression. The promoters employed may be constitutive, tissue-specific, inducible, and/or useful under the appropriate conditions to direct expression of the introduced DNA segment in vivo or in vitro (e.g., in the large-scale production of recombinant proteins and/or peptides). The promoter may be heterologous or endogenous. The identity of tissue-specific promoters or elements, as well as assays to characterize their activity, is well known to those of skill in the art.

One aspect of the disclosure provides a polynucleotide comprising (i) a regulatory sequence that is at least 85% (e.g., 90%, 95%, 96%, 96%, 98%, 99%, and 100%) identical to a promoter or enhancer sequence of a gene selected from the group consisting of SIX1, ATOH1, SOX21, MYO7a, those listed below, and those in Table A and (ii) a transgene operably linked to the regulatory sequence, wherein the transgene is selected from the group consisting of VGLUT3, MYO7A (USH1B), USH1C, CDH23, PCDH15 (USH1F), SANS (USH1G), USH2A, ADGRV1/VLGR1, WHRN (DFNB31), USH3A (CLRN1), HARS, Cx26 (GJB2), Cx30 (GJB6), Cx29 (GJC3), Cx31 (GJB3), ACTG1, FSCN2, RDX, POU4F3, TRIOBP, TPRN, XIRP2, ATOH1, GFI1, CHRNA9, CIB3, CDH23, PCDH15, KNCN, DFNB59, OTOF, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, CABP2, EPS8, EPS8L2, ESPN, ESPNL, PRPH2, STRC, SLC8A2, ZCCHC12, LRTOMT2, LRTOMT1, USH1C, ELFN1, TTC24, DYTN, KCP, CCER2, LRTM2, KCNA10, NTF3, CLRN1, CLRN2, SKOR1, TCTEX1 D1, FCRLB, SLC17A8, GRXCR2, BDNF, SERPINE3, NHLH1, HSP70, HSP90, ATF6, PERK, IRE1, BIP, GJB2, and USH1G. Amino acid sequences and related nucleic acid sequences encoding these polypeptides are known in the art. Listed in Table B below are GenBank accession numbers for some examples.

In some embodiments, the transgene is selected from the group consisting of VGLUT3, MYO7A (USH1B), USH1C, CDH23, PCDH15 (USH1F), SANS (USH1G), USH2A, ADGRV1/VLGR1, WHRN (DFNB31), USH3A (CLRN1), HARS, Cx26 (GJB2), Cx30 (GJB6), Cx29 (GJC3), and Cx31 (GJB3). Exemplary amino acid sequences are shown in Table C below.

A number of genes are known to specifically express in cochlea cells, e.g., hair cells (HCs), supporting cells (SCs), and spiral ganglion neurons (SGN). See the able below. The methods described herein can be used to increase expression of any of these genes in those cells too.

TABLE A

| HCs | SCs | SGNs |
|---|---|---|
| Ocm | Gib2 | Syn |
| Slc26a5 | Lgr5 | NeuN |
| Otof | GFAP | Map2 |
| Atp2a3 | Fgfr3 | Tuj 1 |
| Tpbgl | Tak1 | Calb1 |
| Dnajc5b | Sox21 | Calb2 |
| Myo7a | Sox2 | Nos 1 |
| Myo6 | PLP1 | Runx1 |
| Zfp | CD44 | Prph |
| Tmc1 | Prox1 | Cacna1h |
| Tmc2 | CX30 | Slc6a4 |
| Cabp2 | Aquaporin4 | Grm8 |
| Brip 1 | | Brn3a |
| Zmat3 | | Trim54 |
| Strip2 | | |

One or more of the genes are specifically expressed during cochlea sensory epithelium development and are distinguished by their low or absent expression the adult brain. See, e.g., US20210388045, US20200392516, Ahmed et al., Developmental Cell 22, 377-390, Feb. 14, 2012, Wingard and Zhao, Frontiers in Cellular Neuroscience, volume 9, article 202, 2015, Lan et al., Gene Ther 27(7): 329-337, 2020). All of these references are incorporated by reference in their entireties. The promoters of these genes will allow cochlear epithelial cell selective gene expression.

TABLE B

| Name | GenBank Accession Number |
|---|---|
| VGLUT3 | AJ459241.1 |
| MYO7A (USH1B) | NM_000260.4, NM_001127180.2, NM_001369365.1 |
| USHIC | NM_005709.4, NR_123738.2, NM_153676.4, NM_001297764.2 |
| CDH23 | AY358617.1 |
| PCDH15 (USHIF) | EU718482.1, NM_001142763.2, NM_001142764.2, NM_033056.4 |
| SANS (USH1G) | MG100207.1, NM_173477.5, NM_001282489.3. XM_011524296.2 |
| USH2A | AF055580.2, NM_007123.6, NM_206933.4 |
| ADGRV1/VLGR1 | AF055084.1, NM_032119.4, NR_003149.2 |
| WHRN (DFNB31) | NM_015404.4, NM_001083885.3, NM_001173425.2, |
| | NM_001346890.1 |
| USH3A (CLRN1) | AF388366.1, AF388368.1, AF495717.1, NM_174878.3 |
| HARS | NM_002109.6, NM_001258040.3, NM_001258041.3, |
| | NM_001258042.3 |
| Cx26 (GJB2) | NM_004004.6, MH188083.1, KF638275.1 |
| Cx30 (GJB6) | NM_001110219.3, NM_001110220.3, NM_006783.5, |
| | NM_001110221.3 |
| Cx29 (GJC3) | NM_181538.3 |
| Cx31 (GJB3) | AF052692.1, AF099730.1, NM_024009.3, NM_001005752.2 |
| ACTG1 | NM_001199954.3 |
| FSCN2 | NM_012418.4 |
| RDX | DQ916742.1 |
| POU4F3 | NM_002700.3 |
| TRIOBP | NM_007032.5 |
| TPRN | NM_001128228.3 |
| XIRP2 | NM_152381.6 |
| ATOH1 | NM_005172.2 |
| GFI1 | NM_005263.5 |
| CHRNA9 | NM_017581.4 |
| CIB3 | NM_054113.4 |
| CDH23 | AY358617.1 |
| PCDH15 | NM_001142763.2 |
| KNCN | NM_001322255.2 |
| DFNB59 | DQ365827.1 |
| OTOF | NM_194248.3 |
| MKRN2OS | NM_001195279.2 |
| LHX3 | NM_178138.6 |
| TMC1 | NM_138691.3 |
| MYO15 | NM_016239.4 |
| MYO7A | NM_000260.4 |
| MYO6 | NM_004999.4 |
| MYO3A | NM_017433.5 |
| MYO3B | NM_001083615.4 |
| GRXCR1 | NM_001080476.3 |
| PTPRQ | NM_001145026.2 |
| LCE6A | NM_001128600.2 |
| LOXHD1 | NM_144612.7 |
| ART1. | NM_004314.3 |
| ATP2B2 | NM_001001331.4 |
| CIB2 | NM_006383.4 |
| CACNA2D4 | NM_172364.5 |
| CABP2 | AF170811.1 |
| EPS8 | NM_001413831.1 |
| EPS8L2 | NM_022772.4 |
| ESPN | NM_031475.3 |
| ESPNL | NM_194312.4 |
| PRPH2 | NM_000322.5 |
| STRC | NM_153700.2 |
| SLC8A2 | NM_015063.3 |
| ZCCHC12 | NM_173798.4 |
| LRTOMT2 | NM_001145308 |
| LRTOMT1 | BC012855.1 |
| USHIC | NM_005709.4 |
| ELFN1 | NM_001128636.4 |
| TTC24 | NM_001105669.4 |
| DYTN | NM_001093730.1 |
| KCP | NM_001366122.1 |
| CCER2 | NM_001243212.2 |
| LRTM2 | NM_001039029.3 |
| KCNA10 | NM_005549.2 |
| NTF3 | NM_001102654.2 |
| CLRN1 | NM_174878.3 |
| CLRN2 | NM_001079827.2 |
| SKOR1 | NM_001365915.1 |
| TCTEX1 D1 | BC106885.2 |
| FCRLB | AF329493.1 |
| SLC17A8 | NM_139319.3 |
| GRXCR2 | NM_001080516.2 |
| BDNF | X91251.1 |
| SERPINE3 | NM_001386375.1 |
| NHLH1 | NM_005598.4 |
| HSP70 | L12723.2 |
| HSP90 | NM_001017963.3 |
| ATF6 | AB015856.1 |
| PERK | NM_004836.7 |
| IRE1 | AF059198.1 |
| BIP | X87949.1 |

TABLE C

| SEQ | | |
|---|---|---|
| ID NO | Name | Amino Acid Sequence |
| 1 | VGLUT3 | MPFKAFDTFKEKILKPGKEGVKNAVGDSLGILQRKIDGTTEEEDNIELNEEGRPVQTS |
| | | RPSPPLCDCHCCGLPKRYIIAIMSGLGFCISFGIRCNLGVAIVEMVNNSTVYVDGKPE |
| | | IQTAQFNWDPETVGLIHGSFFWGYIMTQIPGGFISNKFAANRVFGAAIFLTSTLNMFI |
| | | PSAARVHYGCVMCVRILQGLVEGVTYPACHGMWSKWAPPLERSRLATTSFCGSYAGAV |
| | | VAMPLAGVLVQYIGWSSVFYIYGMFGIIWYMFWLLQAYECPAAHPTISNEEKTYIETS |
| | | IGEGANVVSLSKFSTPWKRFFTSLPVYAIIVANFCRSWTFYLLLISQPAYFEEVFGFA |
| | | ISKVGLLSAVPHMVMTIVVPIGGQLADYLRSRQILTTTAVRKIMNCGGFGMEATLLLV |
| | | VGFSHTKGVAISFLVLAVGFSGFAISGFNVNHLDIAPRYASILMGISNGVGTLSGMVC |
| | | PLIVGAMTRHKTREEWQNVFLIAALVHYSGVIFYGVFASGEKQEWADPENLSEEKCGI |
| | | IDQDELAEEIELNHESFASPKKKMSYGATSQNCEVQKKEWKGQRGATLDEEELTSYQN |
| | | EERNFSTIS |
| 2 | MYO7A | MVILQQGDHVWMDLRLGQEFDVPIGAVVKLCDSGQVQVVDDEDNEHWISPQNATHIK |
| | (USH1B), | PMHPTSVHGVEDMIRLGDLNEAGILRNLLIRYRDHLIYTYTGSILVAVNPYQLLSIYS |
| | | PEHIRQYTNKKIGEMPPHIFAIADNCYFNMKRNSRDQCCIISGESGAGKTESTKLILQ |
| | | FLAAISGQHSWIEQQVLEATPILEAFGNAKTIRNDNSSRFGKYIDIHFNKRGAIEGAK |
| | | IEQYLLEKSRVCRQALDERNYHVFYCMLEGMSEDQKKKLGLGQASDYNYLAMGNCITC |
| | | EGRVDSQEYANIRSAMKVLMFTDTENWEISKLLAAILHLGNLQYEARTFENLDACEVL |
| | | FSPSLATAASLLEVNPPDLMSCLTSRTLITRGETVSTPLSREQALDVRDAFVKGIYGR |
| | | LFVWIVDKINAAIYKPPSQDVKNSRRSIGLLDIFGFENFAVNSFEQLCINFANEHLQQ |
| | | FFVRHVFKLEQEEYDLESIDWLHIEFTDNQDALDMIANKPMNIISLIDEESKFPKGTD |
| | | TTMLHKLNSQHKLNANYIPPKNNHETQFGINHFAGIVYYETQGFLEKNRDTLHGDIIQ |
| | | LVHSSRNKFIKQIFQADVAMGAETRKRSPTLSSQFKRSLELLMRTLGACQPFFVRCIK |
| | | PNEFKKPMLFDRHLCVRQLRYSGMMETIRIRRAGYPIRYSFVEFVERYRVLLPGVKPA |
| | | YKQGDLRGTCQRMAEAVLGTHDDWQIGKTKIFLKDHHDMLLEVERDKAITDRVILLQK |
| | | VIRGFKDRSNFLKLKNAATLIQRHWRGHNCRKNYGLMRLGFLRLQALHRSRKLHQQYR |
| | | LARQRIIQFQARCRAYLVRKAFRHRLWAVLTVQAYARGMIARRLHQRLRAEYLWRLEA |
| | | EKMRLAEEEKLRKEMSAKKAKEEAERKHQERLAQLAREDAERELKEKEAARRKKELLE |
| | | QMERARHEPVNHSDMVDKMFGFLGTSGGLPGQEGQAPSGFEDLERGRREMVEEDLDAA |
| | | LPLPDEDEEDLSEYKFAKFAATYFQGTTTHSYTRRPLKQPLLYHDDEGDQLAALAVWI |
| | | TILRFMGDLPEPKYHTAMSDGSEKIPVMTKIYETLGKKTYKRELQALQGEGEAQLPEG |
| | | QKKSSVRHKLVHLTLKKKSKLTEEVTKRLHDGESTVQGNSMLEDRPTSNLEKLHFIIG |
| | | NGILRPALRDEIYCQISKQLTHNPSKSSYARGWILVSLCVGCFAPSEKFVKYLRNFIH |
| | | GGPPGYAPYCEERLRRTFVNGTRTQPPSWLELQATKSKKPIMLPVTFMDGTTKTLLTD |
| | | SATTAKELCNALADKISLKDRFGFSLYIALFDKVSSLGSGSDHVMDAISQCEQYAKEQ |
| | | GAQERNAPWRLFFRKEVFTPWHSPSEDNVATNLIYQQVVRGVKFGEYRCEKEDDLAEL |
| | | ASQQYFVDYGSEMILERLLNLVPTYIPDREITPLKTLEKWAQLAIAAHKKGIYAQRRT |
| | | DAQKVKEDVVSYARFKWPLLFSRFYEAYKFSGPSLPKNDVIVAVNWTGVYFVDEQEQV |
| | | LLELSFPEIMAVSSSRECRVWLSLGCSDLGCAAPHSGWAGLTPAGPCSPCWSCRGAKT |
| | | TAPSFTLATIKGDEYTFTSSNAEDIRDLVVTFLEGLRKRSKYVVALQDNPNPAGEESG |
| | | FLSFAKGDLIILDHDTGEQVMNSGWANGINERTKQRGDFPTDSVYVMPTVTMPPREIV |
| | | ALVTMTPDQRQDVVRLLQLRTAEPEVRAKPYTLEEFSYDYFRPPPKHTLSRVMVSKAR |
| | | GKDRLWSHTREPLKQALLKKLLGSEELSQEACLAFIAVLKYMGDYPSKRTRSVNELTD |
| | | QIFEGPLKAEPLKDEAYVQILKQLTDNHIRYSEERGWELLWLCTGLFPPSNILLPHVQ |
| | | RFLQSRKHCPLAIDCLQRLQKALRNGSRKYPPHLVEVEAIQHKTTQIFHKVYFPDDTD |
| | | EAFEVESSTKAKDFCQNIATRLLLKSSEGFSLFVKIADKVLSVPENDFFFDFVRHLTD |
| | | WIKKARPIKDGIVPSLTYQVFFMKKLWTTTVPGKDPMADSIFHYYQELPKYLRGYHKC |
| | | TREEVLQLGALIYRVKFEEDKSYFPSIPKLLRELVPQDLIRQVSPDDWKRSIVAYFNK |
| | | HAGKSKEEAKLAFLKLIFKWPTFGSAFFEVKQTTEPNFPEILLIAINKYGVSLIDPKT |
| | | KDILTTHPFTKISNWSSGNTYFHITIGNLVRGSKLLCETSLGYKMDDLLTSYISQMLT |
| | | AMSKQRGSRSGK |
| 3 | USH1C, | MDRKVAREFRHKVDFLIENDAEKDYLYDVLRMYHQTMDVAVLVGDLKLVINEPSRLPL |
| | | FDAIRPLIPLKHQVEYDQLTPRRSRKLKEVRLDRLHPEGLGLSVRGGLEFGCGLFISH |
| | | LIKGGQADSVGLQVGDEIVRINGYSISSCTHEEVINLIRTKKTVSIKVRHIGLIPVKS |
| | | SPDEPLTWQYVDQFVSESGGVRGSLGSPGNRENKEKKVFISLVGSRGLGCSISSGPIQ |
| | | KPGIFISHVKPGSLSAEVGLEIGDQIVEVNGVDFSNLDHKEAVNVLKSSRSLTISIVA |
| | | AAGRELFMTDRERLAEARQRELQRQELLMQKRLAMESNKILQEQQEMERQRRKEIAQK |
| | | AAEENERYRKEMEQIVEEEEKFKKQWEEDWGSKEQLLLPKTITAEVHPVPLRKPKYDQ |
| | | GVEPELEPADDLDGGTEEQGEQDFRKYEEGFDPYSMFTPEQIMGKDVRLLRIKKEGSL |
| | | DLALEGGVDSPIGKVVVSAVYERGAAERHGGIVKGDEIMAINGKIVTDYTLAEAEAAL |
| | | QKAWNQGGDWIDLVVAVCPPKEYDDELTFF |
| 4 | CDH23, | MGCHVATSCHVAWLLVLISGCWGQVNRLPFFTNHFFDTYLLISEDTPVGSSVTQLLAQ |
| | | DMDNDPLVFGVSGEEASRFFAVEPDTGVVWLRQPLDRETKSEFTVEFSVSDHQGVITR |
| | | KVNIQVGDVNDNAPTFHNQPYSVRIPENTPVGTPIFIVNATDPDLGAGGSVLYSFQPP |
| | | SQFFAIDSARGIVTVIRELDYETTQAYQLTVNATDQDKTRPLSTLANLAIIITDVQDM |
| | | DPIFINLPYSTNIYEHSPPGTTVRIITAIDQDKGRPRGIGYTIVSGNTNSIFALDYIS |
| | | GVLTLNGLLDRENPLYSHGFILTVKGTELNDDRTPSDATVTTTFNILVIDINDNAPEF |
| | | NSSEYSVAITELAQVGFALPLFIQVVDKDENLGLNSMFEVYLVGNNSHHFIISPTSVQ |
| | | GKADIRIRVAIPLDYETVDRYDFDLFANESVPDHVGYAKVKITLINENDNRPIFSQPL |
| | | YNISLYENVTVGTSVLTVLVSPRFTAGPLSSPGPTVVRHPEGFCPRDLSNQGRRHPQI |
| | | PELCLLVY |
| 5 | PCDH15 | MFRQFYLWTCLASGIILGSLFEICLGQYDDDCKLARGGPPATIVAIDEESRNGTILVD |
| | (USH1F), | NMLIKGTAGGPDPTIELSLKDNVDYWVLMDPVKQMLFLNSTGRVLDRDPPMNIHSIVV |
| | | QVQCINKKVGTIIYHEVRIVVRDRNDNSPTFKHESYYATVNELTPVGTTIFTGFSGDN |
| | | GATDIDDGPNGQIEYVIQYNPDDPTSNDTFEIPLMLTGNIVLRKRLNYEDKTRYFVII |
| | | QANDRAQNLNERRTTTTTLTVDVLDGDDLGPMFLPCVLVPNTRDCRPLTYQAAIPELR |
| | | TPEELNPIIVTPPIQAIDQDRNIQPPSDRPGILYSILVGTPEDYPRFFHMHPRTAELS |
| | | LLEPVNRDFHQKFDLVIKAEQDNGHPLPAFAGLHIEILDENNQSPYFTMPSYQGYILE |
| | | SAPVGATISDSLNLTSPLRIVALDKDIEDTKDPELHLFLNDYTSVFTVTQTGITRYLT |
| | | LLQPVDREEQQTYTFSITAFDGVQESEPVIVNIQVMDANDNTPTFPEISYDVYVYTDM |
| | | RPGDSVIQLTAVDADEGSNGEITYEILVGAQGDFIINKTTGLITIAPGVEMIVGRTYA |
| | | LTVQAADNAPPAERRNSICTVYIEVLPPNNQSPPRFPQLMYSLEISEAMRVGAVLLNL |
| | | QATDREGDSITYAIENGDPQRVFNLSETTGILTLGKALDRESTDRYILIITASDGRPD |
| | | GTSTATVNIVVTDVNDNAPVFDPYLPRNLSVVEEEANAFVGQVKATDPDAGINGQVHY |
| | | SLGNFNNLFRITSNGSIYTAVKLNREVRDYYELVVVATDGAVHPRHSTLTLAIKVLDI |
| | | DDNSPVFTNSTYTVLVEENLPAGTTILQIEAKDVDLGANVSYRIRSPEVKHFFALHPF |
| | | TGELSLLRSLDYEAFPDQEASITFLVEAFDIYGTMPPGIATVTVIVKDMNDYPPVFSK |
| | | RIYKGMVAPDAVKGTPITTVYAEDADPPGLPASRVRYRVDDVQFPYPASIFEVEEDSG |
| | | RVITRVNLNEEPTTIFKLVVVAFDDGEPVMSSSATVKILVLHPGEIPRFTQEEYRPPP |
| | | VSELATKGTMVGVISAAAINQSIVYSIVSGNEEDTFGINNITGVIYVNGPLDYETRTS |
| | | YVLRVQADSLEVVLANLRVPSKSNTAKVYIEIQDENNHPPVFQKKFYIGGVSEDARMF |
| | | TSVLRVKATDKDTGNYSVMAYRLIIPPIKEGKEGFVVETYTGLIKTAMLFHNMRRSYF |
| | | KFQVIATDDYGKGLSGKADVLVSVVNQLDMQVIVSNVPPTLVEKKIEDLTEILDRYVQ |
| | | EQIPGAKVVVESIGARRHGDAFSLEDYTKCDLTVYAIDPQTNRAIDRNELFKFLDGKL |
| | | LDINKDFQPYYGEGGRILEIRTPEAVTSIKKRGESLGYTEGALLALAFIIILCCIPAI |
| | | LVVLVSYRQFKVRQAECTKTARIQAALPAAKPAVPAPAPVAAPPPPPPPPPGAHLYEE |
| | | LGDSSMYEMPQYGSRRRLLPPAGQEEYGEVVGEAEEEYEEEEWARKRMIKLVVDREYE |
| | | TSSTGEDSAPECQRNRLHHPSIHSNINGNIYIAQNGSVVRTRRACLTDNLKVASPVRL |
| | | GGPFKKLDKLAVTHEENVPLNTLSKGPFSTEKMNARPTLVTFAPCPVGTDNTAVKPLR |
| | | NRLKSTVEQESMIDSKNIKEALEFHSDHTQSDDEELWMGPWNNLHIPMTKL |
| 6 | SANS | MAAMKGHMECVRYLDSIAAKQSSLNPKLVGKLKDKAFREAERRIRECAKLQRRHHERM |
| | (USH1G), | ERRYRRELAERSDTLSFSSLTSSTLSRRLQHLALGSHLPYSQATLHGTARGKTKMQKK |
| | | LERRKQGGEGTFKVSEDGRKSARSLSGLQLGSDVMFVRQGTYANPKEWGRAPLRDMFL |
| | | SDEDSVSRATLAAEPAHSEVSTDSGHDSLFTRPGLGTMVFRRNYLSSGLHGLGRTDWA |
| | | ARMGVWMGWERRGVGCRAPPAWTMTAWAVPTACRTAAVGRSCPGMSSI |
| 7 | USH2A, | MNCPVLSLGSGFLFQVIEMLIFAYFASISLTESRGLFPRLENVGAFKKVSIVPTQAVC |
| | | GLPDRSTFCHSSAAAESIQFCTQRFCIQDCPYRSSHPTYTALFSAGLSSCITPDKNDL |
| | | HPNAHSNSASFIFGNHKSCFSSPPSPKLMASFTLAVWLKPEQQGVMCVIEKTVDGQIV |
| | | FKLTISEKETMFYYRTVNGLQPPIKVMTLGRILVKKWIHLSVQVHQTKISFFINGVEK |
| | | DHTPFNARTLSGSITDFASGTVQIGQSLNGLEQFVGRMQDFRLYQVALTNREILEVFS |
| | | GDLLRLHAQSHCRCPGSHPRVHPLAQRYCIPNDAGDTADNRVSRLNPEAHPLSFVNDN |
| | | DVGTSWVSNVFTNITQLNQGVTISVDLENGQYQVFYIIIQFFSPQPTEIRIQRKKENS |
| | | LDWEDWQYFARNCGAFGMKNNGDLEKPDSVNCLQLSNFTPYSRGNVTFSILTPGPNYR |
| | | PGYNNFYNTPSLQESVKATQIRFHFHGQYYTTETAVNLRHRYYAVDEITISGRCQCHG |
| | | HADNCDTTSQPYRCLCSQESFTEGLHCDRCLPLYNDKPFRQGDQVYAFNCKPCQCNSH |
| | | SKSCHYNISVDPFPFEHFRGGGGVCDDCEHNTTGRNCELCKDYFFRQVGADPSAIDVC |
| | | KPCDCDTVGTRNGSILCDQIGGQCNCKRHVSGRQCNQCQNGFYNLQELDPDGCSPCNC |
| | | NTSGTVDGDITCHQNSGQCKCKANVIGLRCDHCNFGFKFLRSFNDVGCEPCQCNLHGS |
| | | VNKFCNPHSGQCECKKEAKGLQCDTCRENFYGLDVTNCKACDCDTAGSLPGTVCNAKT |
| | | GQCICKPNVEGRQCNKCLEGNFYLRQNNSFLCLPCNCDKTGTINGSLLCNKSTGQCPC |
| | | KLGVTGLRCNQCEPHRYNLTIDNFQHCQMCECDSLGTLPGTICDPISGQCLCVPNRQG |
| | | RRCNQCQPGFYISPGNATGCLPCSCHTTGAVNHICNSLTGQCVCQDASIAGQRCDQCK |
| | | DHYFGFDPQTGRCQPCNCHLSGALNETCHLVTGQCFCKQFVTGSKCDACVPSASHLDV |
| | | NNLLGCSKTPFQQPPPRGQVQSSSAINLSWSPPDSPNAHWLTYSLLRDGFEIYTTEDQ |
| | | YPYSIQYFLDTDLLPYTKYSYYIETTNVHGSTRSVAVTYKTKPGVPEGNLTLSYIIPI |
| | | GSDSVTLTWTTLSNQSGPIEKYILSCAPLAGGQPCVSYEGHETSATIWNLVPFAKYDF |
| | | SVQACTSGGCLHSLPITVTTAQAPPQRLSPPKMQKISSTELHVEWSPPAELNGIIIRY |
| | | ELYMRRLRSTKETTSEESRVFQSSGWLSPHSFVESANENALKPPQTMTTITGLEPYTK |
| | | YEFRVLAVNMAGSVSSAWVSERTGESAPVFMIPPSVFPLSSYSLNISWEKPADNVTRG |
| | | KVVGYDINMLSEQSPQQSIPMAFSQLLHTAKSQELSYTVEGLKPYRIYEFTITLCNSV |
| | | GCVTSASGAGQTLAAAPAQLRPPLVKGINSTTIHLKWFPPEELNGPSPIYQLERRESS |
| | | LPALMTTMMKGIRFIGNGYCKFPSSTHPVNTDFTGKCV |
| 8 | ADGRV1/ | MQLCIFCCCCILFYFDLYDFGRGYDFTIQENGLQIDQPPEIGNISIVRIIIMKNDNAE |
| | VLGR1, | GIIEFDPKYTAFEVEEDVGLIMIPVVRLHGTYGYVTADFISQSSSASPGGVDYILHGS |
| | | TVTFQHGQNLSFINISIIDDNESEFEEPIEILLTGATGGAVLGRHLVSRIIIAKSDSP |
| | | FGVIRFLNQSKISIANPNSTMILSLVLERTGGLLGEIQVNWETVGPNSQEALLPQNRD |
| | | IADPVSGLFYFGEGEGGVRTIILTIYPHEEIEVEETFIIKLHLVKGEAKLDSRAKDVT |
| | | LTIQEFGDPNGVVQFAPETLSKKTYSEPLALEGPLLITFFVRRVKGTFGEIMVYWELS |
| | | SEFDITEDFLSTSGFFTIADGESEASFDVHLLPDEVPEIEEDYVIQLVSVEGGAELDL |
| | | EKSITWFSVYANDDPHGVFALYSDRQSILIGQNLIRSIQINITRLAGTFGDVAVGLRI |
| | | SSDHKEQPIVTENAERQLVVKDGATYKVDVVPIKNQVFLSLGSNFTLQLVTVMLVGGR |
| | | FYGMPTILQEAKSAVLPVSEKAANSQVGFESTAFQLMNITAGTSHVMISRRGTYGALS |
| | | VAWTTGYAPGLEIPEFIVVGNMTPTLGSLSFSHGEQRKGVFLWTFPSPGWPEAFVLHL |
| | | SGVQSSAPGGAQLRSGFIVAEIEPMGVFQFSTSSRNIIVSEDTQMIRLHVQRLFGFHS |
| | | DLIKVSYQTTAGSAKPLEDFEPVQNGELFFQKFQTEVDFEITIINDQLSEIEEFFYIN |
| | | LTSVEIRGLQKFDVNWSPRLNLDFSVAVITILDNDDLAGMDISFPETTVAVAVDTTLI |
| | | PVETESTTYLSTSKTTTILQPTNVVAIVTEATGVSAIPEKLVTLHGTPAVSEKPDVAT |
| | | VTANVSIHGTFSLGPSIVYIEEEMKNGTFNTAEVLIRRTGGFTGNVSITVKTFGERCA |
| | | QMEPNALPFRGIYGISNLTWAVEEEDFEEQTLTLIFLDGERERKVSVQILDDDEPEGQ |
| | | EFFYVFLTNPQGGAQIVEGKDDTGFAAFAMVIITGSDLHNGIIGFSEESQSGLELREG |
| | | AVMRRLHLIVTRQPNRAFEDVKVFWRVTLNKTVVVLQKDGVNLMEELQSVSGTTTCTM |
| | | GQTKCFISIELKPEKVPQVEVYFFVELYEATAGAAINNSARFAQIKILESDESQSLVY |
| | | FSVGSRLAVAHKKATLISLQVARDSGTGLMMSVNFSTQELRSAETIGRTIISPAISGK |
| | | DFVITEGTLVFEPGQRSTVLDVILTPETGSLNSFPKRFQIVLFDPKGGARIDKVYGTA |
| | | NITLVSDADSQAIWGLADQLHQPVNDDILNRVLHTISMKVATENTDEQLSAMMHLIEK |
| | | ITTEGKIQAFSVASRTLFYEILCSLINPKRKDTRGFSHFAEVTENFAFSLLTNVTCGS |
| | | PGEKSKTILDSCPYLSILALHWYPQQINGHKFEGKEGDYIRIPERLLDVQDAEIMAGK |
| | | STCKLVQFTEYSSQQWFISGNNLPTLKNKVLSLSVKGQSSQLLTNDNEVLYRIYAAEP |
| | | RIIPQTSLCLLWNQAAASWLSDSQFCKVIEETADYVECACSHMSVYAVYARTDNLSSY |
| | | NEAFFTSGFICISGLCLAVLSHIFCARYSMFAAKLLTHMMAASLGTQILFLASAYASP |
| | | QLAEESCSAMAAVTHYLYLCQFSWMLIQSVNFWYVLVMNDEHTERRYLLFFLLSWGLP |
| | | AFVVILLIVILKGIYHQSMSQIYGLIHGDLCFIPNVYAALFTAALVPLTCLVVVFVVF |
| | | IHAYQVKPQWKAYDDVFRGRTNAAEIPLILYLFALISVTWLWGGLHMAYRHFWMLVLF |
| | | VIFNSLQGLYVFMVYFILHNQMCCPMKASYTVEMNGHPGPSTAFFTPGSGMPPAGGEI |
| | | SKSTQNLIGAMEEVPPDWERASFQQGSQASPDLKPSPQNGATFPSSGGYGQGSLIADE |
| | | ESQEFDDLIFALKTGAGLSVSDNESGQGSQEGGTLTDSQIVELRRIPIADTHL |
| 9 | WHRN | MNAPLDGLSVSSSSTGSLGSAAGAGGGGGAGLRLLSANVRQLHQALTALLSEAEREQF |
| | (DFNB31), | THCLNAYHARRNVFDLVRTLRVLLDSPVKRRLLPMLRLVIPRSDQLLFDQYTAEGLYL |
| | | PATTPYRQPAWGGPDSAGPGEVRLVSLRRAKAHEGLGFSIRGGSEHGVGIYVSLVEPG |
| | | SLAEKEGLRVGDQILRVNDKSLARVTHAEAVKALKGSKKLVLSVYSAGRIPGGYVTNH |
| | | IYTWVDPQGRSISPPSGLPQPHGGALRQQEGDRRSTLHLLQGGDEKKVNLVLGDGRSL |
| | | GLTIRGGAEYGLGIYITGVDPGSEAEGSGLKVGDQILEVNGRSFLNILHDEAVRLLKS |
| | | SRHLILTVKDVGRLPHARTTVDETKWIASSRIRETMANSAGFLGDLTTEGINKPGFYK |
| | | GPAGSQVTLSSLGNQTRVLLEEQARHLLNEQEHATMAYYLDEYRGGSVSVEALVMALF |
| | | KLLNTHAKFSLLSEVRGTISPQDLERFDHLVLRREIESMKARQPPGPGAGDTYSMVSY |
| | | SDTGSSTGSHGTSTTVSSARNTLDLEETGEAVQGNINALPDVSVDDVRSTSQGLSSFK |
| | | PLPRPPPLAQGNDLPLGQPRKLGREDLQPPSSMPSCSGTVFSAPQNRSPPAGTAPTPG |
| | | TSSAQDLPSSPIYASVSPANPSSKRPLDAHLALVNQHPIGPFPRVQSPPHLKSPSAEA |
| | | TVAGGCLLPPSPSGHPDQTGTNQHFVMVEVHRPDSEPDVNEVRALPQTRTASTLSQLS |
| | | DSGQTLSEDSGVDAGEAEASAPGRGRQSVSTKSRSSKELPRNERPTDGANKPPGLLEP |
| | | TSTLVRVKKSAATLGIAIEGGANTRQPLPRIVTIQRGGSAHNCGQLKVGHVILEVNGL |
| | | TLRGKEHREAARIIAEAFKTKDRDYIDFLVTEFNVML |
| 10 | USH3A | MQALQQQPVFPDLLKAIPVSIHVNVILFSAILIVLTMVGTAFFMYNAFGKPFETLHGP |
| | (CLRN1), | LGLYLLSFISGSCGCLVMILFASEVKIHHLSEKIANYKEGTYVYKTQSEKYTTSFWLT |
| | | KGHS |
| 11 | HARS, | MAERAALEELVKLQGERVRGLKQQKASAELIEEEVAKLLKLKAQLGPDESKQKFVLKT |
| | | PKGTRDYSPRQMAVREKVFDVIIRCFKRHGAEVIDTPVFELKETLMGKYGEDSKLIYD |
| | | LKDQGGELLSLRYDLTVPFARYLAMNKLTNIKRYHIAKVYRRDNPAMTRGRYREFYQC |
| | | DFDIAGNFDPMIPDAECLKIMCEILSSLQIGDFLVKVNDRRILDGMFAICGVSDSKFR |
| | | TICSSVDKLDKVSWEEVKNEMVGEKGLAPEVADRIGDYVQQHGGVSLVEQLLQDPKLS |
| | | QNKQALEGLGDLKLLFEYLTLFGIDDKISFDLSLARGLDYYTGVIYEAVLLQTPAQAG |
| | | EEPLGVGSVAAGGRYDGLVGMFDPKGRKVPCVGLSIGVERIFSIVEQRLEALEEKIRT |
| | | TETQVLVASAQKKLLEERLKLVSELWDAGIKAELLYKKNPKLLNQLQYCEEAGIPLVA |
| | | IIGEQELKDGVIKLRSVTSREEVDVRREDLVEEIKRRTGQPLCIC |
| 12 | Cx26 | MDWGTLQTILGGVNKHSTSIGKIWLTVLFIFRIMILVVAAKEVWGDEQADFVCNTLQP |
| | (GJB2), | GCKNVCYDHYFPISHIRLWALQLIFVSTPALLVAMHVAYRRHEKKRKFIKGEIKSEFK |
| | | DIEEIKTQKVRIEGSLWWTYTSSIFFRVIFEAAFMYVFYVMYDGFSMQRLVKCNAWPC |
| | | PNTVDCFVSRPTEKTVFTVFMIAVSGICILLNVTELCYLLIRYCSGKSKKPV |
| 13 | Cx30 | MDWGTLHTFIGGVNKHSTSIGKVWITVIFIFRVMILVVAAQEVWGDEQEDFVCNTLQP |
| | (GJB6), | GCKNVCYDHFFPVSHIRLWALQLIFVSTPALLVAMHVAYYRHETTRKFRRGEKRNDFK |
| | | DIEDIKKQKVRIEGSLWWTYTSSIFFRIIFEAAFMYVFYFLYNGYHLPWVLKCGIDPC |
| | | PNLVDCFISRPTEKTVFTIFMISASVICMLLNVAELCYLLLKVCFRRSKRAQTQKNHP |
| | | NHALKESKQNEMNELISDSGQNAITGFPS |
| 14 | Cx29 | MCGRFLRRLLAEESRRSTPVGRLLLPVLLGFRLVLLAASGPGVYGDEQSEFVCHTQQP |
| | (GJC3), | GCKAACFDAFHPLSPLRFWVFQVILVAVPSALYMGFTLYHVIWHWELSGKGKEEETLI |
| | | QGREGNTDVPGAGSLRLLWAYVAQLGARLVLEGAALGLQYHLYGFQMPSSFACRREPC |
| | | LGSITCNLSRPSEKTIFLKTMFGVSGFCLLFTFLELVLLGLGRWWRTWKHKSSSSKYF |
| | | LTSESTRRHKKATDSLPVVETKEQFQEAVPGRSLAQEKQRPVGPRDA |
| 15 | Cx31 | MDWKTLQALLSGVNKYSTAFGRIWLSVVFVFRVLVYVVAAERVWGDEQKDFDCNTKQP |
| | (GJB3), | GCTNVCYDNYFPISNIRLWALQLIFVTCPSLLVILHVAYREERERRHRQKHGDQCAKL |
| | | YDNAGKKHGGLWWTYLFSLIFKLIIEFLFLYLLHTLWHGFNMPRLVQCANVAPCPNIV |
| | | DCYIARPTEKKIFTYFMVGASAVCIVLTICELCYLICHRVLRGLHKDKPRGGCSPSSS |
| | | ASRASTCRCHHKLVEAGEVDPDPGNNKLQASAPNLTPI |

In one embodiment, the transgene encodes a human vesicular glutamate transporter 3 (VGLUT3 gene) protein. An exemplary human VGLUT3 amino acid sequence (GenBank: AJ459241.1) (SEQ ID NO: 1) is shown in Table B. Listed below is its coding nucleic acid sequence (Takamori et al., EMBO Rep. 3 (8), 798-803 (2002)).

| SEQ ID NO: 16: | |
|---|---|
| 1 | tcttggaaga tccgagctgg gtttcatctc ctttttgatt ttgagtagtt ccctccacga |
| 61 | gaactgactt ccaggtgttc accaagggaa acaaggtggt tctcacactg gaaatgagga |
| 121 | aggatgacag tttttgagac tgactgttaa cggctcagag gtgcccctca ttcaaaatgc |
| 181 | cttttaaagc atttgatacc ttcaaagaaa aaattctgaa acctgggaag gaaggagtga |
| 241 | agaacgccgt gggagattct ttgggaattt tacaaagaaa aatcgatggg acaactgagg |
| 301 | aagaagataa cattgagctg aatgaagaag gaaggccggt gcagacgtcc aggccaagcc |
| 361 | ccccactctg cgactgccac tgctgcggcc tccccaagcg ttacatcatt gctatcatga |
| 421 | gtgggctggg attctgcatt tcctttggga tccggtgcaa tcttggagtt gccattgtgg |
| 481 | aaatggtcaa caatagcacc gtatatgttg atggaaaacc ggaaattcag acagcacagt |
| 541 | ttaactggga tccagaaaca gtgggcctta tccatggatc ttttttctgg ggctatatta |
| 601 | tgacacaaat tccaggtggt ttcatttcaa acaagtttgc tgctaacagg gtctttggag |
| 661 | ctgccatctt cttaacatcg actctgaaca tgtttattcc ctctgcagcc agagtgcatt |
| 721 | acggatgcgt catgtgtgtc agaattctgc aaggtttagt ggagggtgtg acctacccag |
| 781 | cctgccatgg gatgtggagt aagtgggcac cacctttgga gagaagccga ctggccacaa |
| 841 | cctctttttg tggttcctat gcaggggcag tggttgccat gcccctggct ggggtgttgg |
| 901 | tgcagtacat tggatggtcc tctgtctttt atatttatgg catgtttggg attatttggt |
| 961 | acatgttttg gctgttgcag gcctatgagt gcccagcagc tcatccaaca atatccaatg |
| 1021 | aggagaagac ctatatagag acaagcatag gagagggggc caacgtggtt agtctaagta |
| 1081 | aatttagtac cccatggaaa agatttttca catctttgcc ggtttacgca atcattgtgg |
| 1141 | caaatttttg cagaagctgg accttttatt tgctcctcat aagtcagcct gcttattttg |
| 1201 | aagaggtctt tggatttgca ataagtaagg tgggtctctt gtcagcagtc ccacacatgg |
| 1261 | ttatgacaat cgttgtacct attggaggac aattggctga ttatttaaga agcagacaaa |
| 1321 | ttttaaccac aactgctgtc agaaaaatca tgaactgtgg aggttttggc atggaggcaa |
| 1381 | ccttactcct ggtggttggc ttttcgcata ccaaaggggt ggctatctcc tttctggtac |
| 1441 | ttgctgtagg atttagtggc ttcgctattt caggttttaa tgtcaaccac ctggacattg |
| 1501 | ccccacgcta tgccagcatt ctcatgggga tctcaaacgg agtgggaacc ctctctggaa |
| 1561 | tggtctgtcc cctcattgtc ggtgcaatga ccaggcacaa gacccgtgaa gaatggcaga |
| 1621 | atgtgttcct catagctgcc ctggtgcatt acagtggtgt gatcttctat ggggtctttg |
| 1681 | cttctgggga gaaacaggag tgggctgacc cagagaatct ctctgaggag aaatgtggaa |
| 1741 | tcattgacca ggacgaatta gctgaggaga tagaactcaa ccatgagagt tttgcgagtc |
| 1801 | ccaaaaagaa gatgtcttat ggagccacct cccagaattg tgaagtccag aagaaggaat |
| 1861 | ggaaaggaca gagaggagcg acccttgatg aggaagagct gacatcctac cagaatgaag |
| 1921 | agagaaactt ctcaactata tcctaatgtc tgagaggcac ttctgtcttc tccttacttt |
| 1981 | agaaacagaa agtatccata cctattgcct ttcttgtagc ccagcttgcc agaggtccaa |
| 2041 | atattgggag gggagaagat ctaaccagca acagggaaaa gagaaatatt atctttcaat |
| 2101 | gacatgtata ggtaaggagc tgcgctcagt tgataacata gttgataata catatttttt |
| 2161 | gaattgacag ttgacccttc tctcaaagag ctaaacttat tcagaaagga atgactagaa |
| 2221 | gaaaaaggag acaataccat gttgttcaaa gaaacattga aggaaattgg gatgtttggc |
| 2281 | cagaaggaat gtaaacagta gtagtagctg ccaccacatc tctagggtag ccatgcagag |
| 2341 | gagggcttca tattcccaat aaaccccacg ttgtggcagg tgctttataa acactcttat |
| 2401 | ttaatctcca cacctttatg acacacattt cttatcccca ttttacaacc aaggcatcta |
| 2461 | aagcaacaag aaatgaactt gcccaaggtc atctgccagg gtcagtgctg agactgttga |
| 2521 | agctctcaat aggtggcagt tttagggaag atttccattc agtgtaggga agacatttgt |
| 2581 | aataatgaaa actgaaaatg gagtaattgt gagtaactca ccactttagc aggtgttggg |
| 2641 | gaagggaaac atttgggttg atgaggcaga ggggattcaa atgtgtgaga ggctagattc |
| 2701 | aaagaccctc agtgttctat gttatctgaa gagtcaaatg gttttgtgac tccatagttt |
| 2761 | ttaaagtaat aagggtcaaa gactacatca gagattcaaa taggttttta aagaaaagct |
| 2821 | aagcaagaga gccaaatttt tagaaatctg atggtcaaaa tagctgaaag cagtaaacaa |
| 2881 | gagattggct attaaatttc aactttccat aatattaaga atgtagctaa atgatgtccc |
| 2941 | aaactactta caaactttta agacatttaa taatttaaga agtaggttca tgtgttttct |
| 3001 | taggtaaagt tcttctgaaa gaattttcta tttttaaaaa atgtatctct ttagcctttt |
| 3061 | ctgctggaga ttatattagg aagtttcatc agattgtata aaattatgat tttgtatcaa |
| 3121 | aagtattcat gatgactcta tttggaatga tattcaggga aatcacaata atatagcagt |
| 3181 | agttatacag agaaatacta caatgaaaac atttggggca attagaccta cagttactgt |
| 3241 | tgaaaaattc acctttgatt gcataaggca attacatgga tacttttaga tatatttaaa |
| 3301 | attttaacat tggcatctaa agtgttattt gaaaataaaa ttattttcct gttcattgat |
| 3361 | tttaaacatt ttattcctac tttcagaaga aaaatataat acggaaaaaa ttatagattt |
| 3421 | acttgtagct tattattgta aagtgttttt tttttttttc taatttctcc cacatgtatt |
| 3481 | tctggtcccc agtgatacta gctgagttgt agtgtatttt ataaatggaa taatcttggg |
| 3541 | gaaaaattgc gattcttcat taaataatat tctttatgtc actagcatac aatttatgtt |
| 3601 | agtagacatc tttaaatctc tttaatgagt gaatccatgc aagccccata aaacagttcc |
| 3661 | tagcatgcag aaaatgccca cgtaaatagc tgtcatcatc attatotttt aacattttgg |
| 3721 | gggactttcc agttgaaaag aaaacatgct atgtcatttt tatccattat ccctggaact |
| 3781 | tattgtgaaa gttgtgctgt tttctaagta aaataaaaaa taaaaaatta gcaattta |

In one embodiment, the promoter or enhancer sequence is a MYO7a promoter or enhancer. A MYO7a (or myosin 7A) promoter or enhancer refers to a regulatory polynucleotide sequence comprising or consisting of a nucleic acid sequence sufficient to direct expression of a downstream polynucleotide in an outer or inner hair cell, a vestibular hair cell, a spiral ganglion, or a vestibular ganglion and having at least about 85% sequence identity to any of the following nucleotide sequences. Examples can also comprise or consist of any of the following sequences or a functional fragment thereof. See, e.g., US 20200392516.

| MYO7A Promoter 1 (SEQ ID NO: 17): | |
|---|---|
| gactttcctgtgtccctctgtccccctggggggaagggacaatggaggaatattccccaggcctgggcgactgtc | |
| cgctggtcagagggaaggccccgctgcctgcccggtgcacctgtgagctgagattgtggggatcattcagtcatt | |
| cctttattcagtagatctgtacggggctggctttgtgccaaggcctgccccgggcacctgggatgtggggaacca | |
| ggcatagcaatccctgctctgggacagtctacaaacgagcagcgagaacaaagacatactctggtcatgacagac | |
| actaccccgcacattactgatggggcagtgtgggcctgctgggttagagacggcctactccctgatctgtgcatg | |
| aagggctgggaagaggagatgggccttcggggcacaggaaggtgtggggggaccagagcggaggctgctgagggc | |
| agatgcaggcatctagggccaggaagctggtgtggtggtggggagaagcagggttgggatgactgaaagcagagt | |
| ggacgactcaatgaaaaacgacttgggagacggggtgagtgcagtgtgagggcaaagtgaaccgagatgcctcgc | |
| tttcttcatgggactgagaaatgggtattgcaaatcgtgtgatctgcttagcatagtgcctggttcatagcatgg | |
| ctgaacacaatgtagctgtcatcctggtgtttggaggccgccctgtcccccataacctgcccacctctctgcccc | |
| attcccacagctcactgcattttgtccctgctgccagtcgtgtgccatgggctctcctgtgctcccccggggtct | |
| catttccccatccgtgtagggctgacgccactggccccgcccagcctgggaggatggagaggggggacttgagga | |
| gtggccagtactgggtttcggcagcaggtgcaaatcccaggtggggactgtgttccagggactcctggactcctg | |
| ttaggccagcaggctaaggggcagacggcatcctggggtcttcctgggacagggtcgtcgggtctgaggggaccc | |
| aggccctagcagaagctgactcctgtcacggtcacttcccagcggtgctcagcccgcctgacggaagcagctggg | |
| cagtggggcctgtgaccagcaggcggcgctggcgagctatagcgcccctttgggcccccaccccaccttctgggc | |
| agcttccctgcagacaccccagttatgggggctagggacccaaaagagacatccttctgccacccagagctgccc | |
| tggcgaggtgcactatggggccgccgacagctgcgtggctgccgagggcggaaaggagaaactgtcatgtcccga | |
| tagggccgcgcgaggtctccatcctcgacaacgctaataacaaagacgtgtgctcctctttgcttggttcccccc | |
| actcctttaaatcacagatttcacttcagtttatctgtgtcgctgtcacacgtggggtggctcccagtcagctgg | |
| tttggcaaagtttctggatgattacggaataacatgtgtccccaacccgcagagcaggttgtgggggcaatgttg | |
| cattgaccagcgtcagagaacacacatcagaggcaagggtgggtgtgcaggagggagaaggcgcagaaggcaggg | |
| ctttagctcagcactctccctcctgccatgctctgcctgaccgttccctctctgagtcccaaacagccaggtaga | |
| ggaggaagaaatggggctgagaccccagcacatcagtgattaagtcaggatcaggtgcggtttcctgctcaggtg | |
| ctgagacagcaggcggtgtcctgcaaacaacaggaggcacctgaagctagcctggggggcccacgcccaggtgcg | |
| gtgcattcagcagcacagccagagacagaccccaatgaccccgcctccctgtcggcagccagtgctctgcacaga | |
| gccctgagcagcctctggacattagtcccagccccagcacggcccgtcccccacgctgatgtcaccgcacccaga | |
| ccttggaggccccctccggctccgcctcctgggagaaggctctggagtgaggaggggagggcagca | |
| MYO7A Promoter 2 (SEQ ID NO: 18) | |
| gtaaggatgggctgcccactgtcctgggcattgggaggggtttggatgtggaggagtcatggacttgagctacct | |
| ctagagcctctgccccacagccacttgctcctgggactgggcttcctgccacccttgagggctcagccaccacag | |
| ccactgaatgaaactgtcccgagcctgggaagatggatgtgtgtcccctggaggagggaagagccaaggagcatg | |
| ttgtccatcgaatcttctctgagctggggctggggttagtggcatcctggggccaggggaatagacatgctgtgg | |
| tggcagagagaagagtccgttctctctgtctcctttgctttctctctgacactctttatctccgtttttggataa | |
| gtcacttccttcctctatgccccaaatatcccatctgtgaaatgggagtatgaagccccaacagccagggttgta | |
| gtggggaagaggtaaaatcaggtatagacatagaaatacaaatacagtctatgccccctgttgtcagttggaaaa | |
| gaaattaacttgaaggtggtctagttctcatttttagaaatgaaatgtctgtctggtcattttaaaatgtggccc | |
| ttaaatttcacgccctcaccactctcccccatcccttggagccccatgtctctagtgaaagcactggctctgccc | |
| ccagccctcatggctcatgctggcatagggcgcctgctccacagcctgggcaccatcttcagacaagtgcccggt | |
| ggcaactgcctgctggccctgttgaatccacatctccaccaggcatccagactagttcaggtctctggaaggact | |
| gtgggtttgctgtgtcccagagctccagggcaggggtcagggctcggatgtcgggcagtgtcatgggcagaggat | |
| cgaatgccccggcggctctgaatgggcccttgtgaaaaattgatgcgcattctaggagacaggttgggagccaga | |
| ggggcctcataccagggtctgtaggctggggctgccttttaagctccttcctgaggccgtctctgggtctggccc | |
| tgtgctggacaaggctggagacaaggcaatgtctcagaccctctcccattggccacatcctgccctggatcaact | |
| cgccaactttgggggcagaggtgggactgacccttaccctgacaacataatgcatatagtcaaaatgggataaag | |
| gggaatatagaggctcttggcagcttgggagtggtcagggaaggcttcctggaggaggtatcatctgaactgagc | |
| catgaaccataagtggaaattcactagtcaaaatttcaggtagaagggccagtgtgtgaaggccaggagatggca | |
| agagctggcgtatttcaggaacagtgagtcactgaggatgtccaagtataagggtaggaaagggagtgagcagtg | |
| agagaaaagaccgaggcatcagcaggggccagattgtgctgggcctagcggggcgggcccgggcccgggcccagg | |
| cccaggtgcggtgcattcagcagcacagccagagacagaccccaatgaccctgcctccccgtcagcagccagtgc | |
| tctgcacagagccatcctgagggcagtgggtgctcttgagaggtttcaggcagggtgtgctgtgagcaggtcatg | |
| cccagcccttgaccttctgctcagtcaggcttgtccttgtcacccacattcctggggcagtccctaagctgagtg | |
| ccggagattaagtcctagtcctaaatttgctctggctagctgtgtgaccctgggcaagtcttggtccctctctgg | |
| gcccctttgccgtaggtccctggtggggccagacttgctactttctaggagccctttgggaatctctgaatgaca | |
| gtggctgagagaagaattcagctgctctgggcagtggtgctggtgacagtggctgaggctcaggtcacacaggct | |
| gggcagtggtcagagggagagaagccaaggagggttcccttgagggaggaggagctggggctttgggaggagccc | |
| aggtgaccccagccaggctcaaggcttccagggctggcctgcccagaagcatgacatggtctctctccctgcaga | |
| MYO7A Promoter 3 (SEQ ID NO: 19) | |
| ttctttcttgacagtttttttattctttcatcattttaaatatgccatcctactgccctctggccgccgtggttt | |
| ctgatgagaaatcagccgagactcatactaacgattcgttgtatagaatgagtcacctctctgttgctgctttca | |
| agagtctctgtctttgccacttgtggagaagaggcctattcctccagctcaagggtggtctcagtagtcgttctg | |
| gaaccaggcaaggtcaggttcttagcaccttgcagggtctcccccaggggacctctctttagccaccagccgcca | |
| gacttactttacaatctcttccctctctcctctgattctctgagtttgctgttccctcttcctgggatgcccttt | |
| cccctaatgtctgcctaaaaaaaaaatcttaaaacccagctgtcacccctaacacgactcccttagttaaccatg | |
| gctggcactcatatacatttggtctaacaggcaccaatctaggtgcattacgcatcttaacctgttaaatcctca | |
| cgagcacgtggaagcagtgtcattttgtcacctgacggatgaggaaagaaaggaacatagaggttgaggaacttg | |
| cccaaggtaacacagcttgtgagtggctgagctgggattgaaactaagccatctgactccagggtccaaactctt | |
| agtggccatgctatcctatgtctccgggactgatctgcctcttatctggtggtaccatccttatctgtgcctggg | |
| tctgtccaccacaccaggctgggagctctgaggacatgtcttgttaacagtgtcctctgtactttagtctgaacc | |
| tgtactcagtgaaggtcggttatatgaactcgtggtagaatgcctggaagctcaccaccagcacttccttgtgtc | |
| tgaggtctgggtcctgtcccaatgtactgctatagatgggcggggagagataggtgtgcctgttttcggcttgac | |
| tgggggaaggacctctcctgagagcggtgtccctgagaggtcacaggaagtgagtgggaatcactgatcagtcag | |
| cttcgtgctggacagcagagccctgggttcccaaggctgggacagggagtggggtagaactgggcccatcctggg | |
| tggctctgagccctgtgtgaggcatcaccgtctttttcgttgccattccccccacccagtcccccgcccccactc | |
| tctgccgttgttcattcgttggggagcagttggcttccaggatactaggcaatgcagagagccacaaatgcagac | |
| tggaggccttgaaaagagaagaggaagaaaaaggaggaaagaggggaggaggacaaagataggcagggcaaggcg | |
| agaaggagggaatgggagaggagtcacagggcctgggccttccggaccctcgggatgggacggtggcctctggct | |
| cacagcccagcgtggaccgacgcctggtgtccagccctgtgtggctgctaggacacaggaggccagcttggccca | |
| ttgcagggagagctctgagccatcgaggtggttcagctgtcctggggagttcaggggaccccacaccaggcctcc | |
| aaggcctcttctgcaggatggacgtgggctgctatttgacctcgggggctgcgggaggaggacatagcctcttca | |
| ggggctttcccccaaacttaccccagaaggaaactgtatctgccctcactccccagaggcagccagggagccaga | |
| aatatcttcaatgcatctctcctttagtcttaaaaaccccgggagtttaccgcgttctagtccataacatcctcg | |
| tgtttccttccatggaaaacatagaaaaaaacatgacgattttttccaaaactgccgtcaggtgcggtgccaggt | |
| gccacgtttatcccgggcctaccgccgcggtgccagtgaagccgcctgggattccgggcaaagtggggctgtggg | |
| gggttcggccgagacggagggggccggggtggcgcgggcgccccctcgccgtcccgcccgcgggcgtcacctaag | |
| ccgccgttgccatgggcccgc | |
| MYO7A Promoter 4 (SEQ ID NO: 20) | |
| agacacccca gttatggggg ctagggaccc aaaagagaca tccttctgcc acccagagct | 60 |
| gccctggcga ggtgcactat ggggccgccg acagctgcgt ggctgccgag ggcggaaagg | 120 |
| agaaactgtc atgtcccgat agggccgcgc gaggtctcca tcctcgacaa cgctaataac | 180 |
| aaagacgtgt gctcctcttt gcttggttcc ccccactcct ttaaatcaca gatttcactt | 240 |
| cagtttatct gtgtcgctgt cacacgtggg gtggctccca gtcagctggt ttggcaaagt | 300 |
| ttctggatga ttacggaata acatgtgtcc ccaacccgca gagcaggttg tgggggcaat | 360 |
| gttgcattga ccagcgtcag agaacacaca tcagaggcaa gggtgggtgt gcaggaggga | 420 |
| gaaggcgcag aaggcagggc tttagctcag cactctccct cctgccatgc tctgcctgac | 480 |
| cgttccctct ctgagtccca aacagccagg tagaggagga agaaatgggg ctgagacccc | 540 |
| agcacatcag tgattaagtc aggatcaggt gcggtttcct gctcaggtgc tgagacagca | 600 |
| ggcggtgtcc tgcaaacaac aggaggcacc tgaagctagc ctggggggcc cacgcccagg | 660 |
| tgcggtgcat tcagcagcac agccagagac agaccccaat gaccccgcct ccctgtcggc | 720 |
| agccagtgct ctgcacagag ccctgagcag cctctggaca ttagtcccag ccccagcacg | 780 |
| gcccgtcccc cacgctgatg tcaccgcacc cagaccttgg aggccccctc cggctccgcc | 840 |
| tcctgggaga aggctctgga gtgaggaggg gagggcagca gtgctggctg gacagctgct | 900 |
| ctgggcagga gagagaggga gagacaagag acacacacag agagacggcg aggaagggaa | 960 |
| agacccagag ggacgcctag aacgagactt ggagccagac agaggaagag gggacgtgtg | 1020 |
| tttgcagact ggctgggccc gtgacccagc ttcctgagtc ctccgtgcag gtggcagctg | 1080 |
| taccaggctg gcaggtcact gagagtgggc agctgggccc caggtaagga tgggctgccc | 1140 |
| actgtcctgg gcattgggag gggtttggat gtggaggagt catggacttg agctacctct | 1200 |
| agagcctctg ccccacagcc acttgctcct gggactgggc ttcctgccac ccttgagggc | 1260 |
| tcagccacca cagccactga atgaaactgt cccgagcctg ggaagatgga tgtgtgtccc | 1320 |
| ctggaggagg gaagagccaa ggagcatgtt gtccatcgaa tcttctctga gctggggctg | 1380 |
| gggttagtgg catcctgggg ccaggggaat agacatgctg tggtggcaga gagaagagtc | 1440 |
| cgttctctct gtctcctttg ctttctctct gacactcttt atctccgttt ttggataagt | 1500 |
| cacttccttc ctctatgccc caaatatccc atctgtgaaa tgggagtatg aagccccaac | 1560 |
| agccagggtt gtagtgggga agaggtaaaa tcaggtatag acatagaaat acaaatacag | 1620 |
| tctatgcccc ctgttgtcag ttggaaaaga aattaacttg aaggtggtct agttctcatt | 1680 |
| tttagaaatg aaatgtctgt ctggtcattt taaaatgtgg cccttaaatt tcacgccctc | 1740 |
| accgctctcc cccatccctt ggagccccat gtctctagtg aaagcactgg ctctgccccc | 1800 |
| agccctcatg gctcatgctg gcatagggcg cctgctccac agcctgggca ccatcttcag | 1860 |
| acaagtgccc ggtggcaact gcctgctggc cctgttgaat ccacatctcc accaggcatc | 1920 |
| cagactagtt caggtctctg gaaggaccgt gggtttgctg tgtcccagag ctccagggca | 1980 |
| ggggtcaggg ctcggatgtc gggcagtgtc atgggcagag gatcgaatgc cccggcggct | 2040 |
| ctgaatgggc ccttgtgaaa aattgatgcg cattctagga gacaggttgg gagccagagg | 2100 |
| ggcctcatac cagggtctgt aggctggggc tgccttttaa gctccttcct gaggccgtct | 2160 |
| ctgggtctgg ccctgtgctg gacaaggctg gagacaaggc aatgtctcag accctctccc | 2220 |
| attggccaca tcctgccctg gatcaactcg ccaactttgg gggcagaggt gggactgacc | 2280 |
| cttaccctga caacataatg catatagtca aaatgggata aaggggaata tagaggctct | 2340 |
| tggcagcttg ggagtggtca gggaaggctt cctggaggag gtatcatctg aactgagcca | 2400 |
| tgaaccataa gtggaaattc actagtcaaa atttcaggta gaagggccag tgtgtgaagg | 2460 |
| ccaggagatg gcaagagctg gcgtatttca ggaacagtga gtcactgagg atgtccaagt | 2520 |
| ataagggtag gaaagggagt gagcagtgag agaaaagacc gaggcatcag caggggccag | 2580 |
| attgtgctgg gcctagcggg gcgggcccgg gcccgggccc aggcccaggt gcggtgcatt | 2640 |
| cagcagcaca gccagagaca gaccccaatg accctgcctc cccgtcagca gccagtgctc | 2700 |
| tgcacagagc catcctgagg gcagtgggtg ctcttgagag gtttcaggca gggtgtgctg | 2760 |
| tgagcaggtc atgcccagcc cttgaccttc tgctcagtca ggcttgtcct tgtcacccac | 2820 |
| attcctgggg cagtccctaa gctgagtgcc ggagattaag tcctagtcct aaatttgctc | 2880 |
| tggctagctg tgtgaccctg ggcaagtctt ggtccctctc tgggcccctt tgccgtaggt | 2940 |
| ccctggtggg gccagacttg ctactttcta ggagcccttt gggaatctct gaatgacagt | 3000 |
| ggctgagaga agaattcagc tgctctgggc agtggtgctg gtgacagtgg ctgaggctca | 3060 |
| ggtcacacag gctgggcagt ggtcagaggg agagaagcca aggagggttc ccttgaggga | 3120 |
| ggaggagctg gggctttggg aggagcccag gtgaccccag ccaggctcaa ggcttccagg | 3180 |
| gctggcctgc ccagaagcat gacatggtct ctctccctgc a | 3221 |

In one embodiment, the promoter or enhancer sequence is a MYO6 promoter or enhancer. A MYO6 (or myosin 6) promoter or enhancer refers to a regulatory polynucleotide sequence comprising or consisting of a nucleic acid sequence sufficient to direct expression of a downstream polynucleotide in an outer or inner hair cell, a vestibular hair cell, a spiral ganglion, or a vestibular ganglion and having at least about 85% sequencing identity to the following nucleotide sequence. Examples may comprise or consist of the following sequence or a functional fragment thereof. See, e.g., US 20200392516.

| MYO6 promoter (SEQ ID NO: 25): | |
|---|---|
| tgcaagaacc ctcactggct gaactatctt gccagcccct tattttgttt tcatattaac | 60 |
| ctcttttttc tagtaaagga gatgtttgct ctcaaatttg cataggaatg taatatttaa | 120 |
| tttaaaaaga tgacccacat atgaccttat aaggacagta aaattaaaca accggaaaga | 180 |
| taaagcgggc cagttggctc agttctataa aaccagccca caaggattgt cactattctt | 240 |
| aggcttgcgc gggctacatg atgagttcca ggactgcctg gttacagacc gagactctct | 300 |
| caagagtcca gataaacaac aacaaagggg gcgaggtgga aatacagggg ctgtaagaag | 360 |
| taaatatgat atctgcatgg gaggctagcc agagaagaaa aaattttctt ccgtggttca | 420 |
| atcctccaag ggctgaacag gaagttgacg caggcaggtg aggagcacga gcctagatgg | 480 |
| gctgcggtgc cacccttaat ccccacaagc gagttcctcc gcaattcgcc tgtcccactc | 540 |
| tcaacttttc ttcaactgac tctttgctgt ggtccctcgc tgtggcagtg gaaacaacta | 600 |
| ccactgcgag gtagggaatg tcatgagggg ctacctgcag cccttggctt gcagggatgc | 660 |
| agggatgcgg tcggaacctg aggccccgcc cttctcttgc cccacgccat taggccacgc | 720 |
| ccctacccag cactccttca accaccccct tccccggcgc ctcatgaggt cccgcccctc | 780 |
| tcaaccctag ctcttgaggc ctccccttca cagccgcccc ggcgttcctt gacttgaggc | 840 |
| cacgtccctc tgctccttca ttcccaagac cctacgcttt gcgagtcctc cctgtcctgc | 900 |
| tgcctaggac cccgcccctc tcagcccttc tgccccaaga ccccgcccct taggctgttc | 960 |
| ccgcccactg gccaatgaag acccgccctt tctttagccg ccccgccccg gtcccacaaa | 1020 |
| atcccgcctc cggccccgcc tcccgccccc ttgggcgctc cgtagcagtg acgtgcgcag | 1080 |
| gctgggcact ctgcagggct ctctggccgg cgggtggaga ccgatccggg atctgtccca | 1140 |
| gcaggaagcg tatcccggcc gccgtcgtgc tgtcgtctcc ggtgctcgct ctcggccgcg | 1200 |
| gtgtcgcgct tgcccttcgc gcccgcagcc cggcagcctc tc | 1242 |

In one embodiment, the promoter or enhancer sequence is an ATOH1 promoter or enhancer. An ATOH1 promoter or enhancer is meant a regulatory polynucleotide sequence comprising or consisting of a nucleic acid sequence sufficient to direct expression of a downstream polynucleotide in an outer or inner hair cell, a vestibular hair cell, a spiral ganglion, or a vestibular ganglion and having at least about 85% sequencing identity to the following nucleotide sequence. Examples may comprise or consist of the following sequence or a functional fragment thereof. See, e.g., US 20190010449.

ATHO1 Promoter 1 (SEQ ID NO: 21)
aaagttctacttaagcatctacagctactagcaattattttctcaagaagcacattttatatggtagcactattt
atagtaacagcattattagtcgatttcaataacacccacttcaatcaccaaacggagaattttctgcccaaaaaa
aaaaaaatacattagatggccttttctttttctttgttctcatggatggttttcagattaggtttgttttttgtt
gttgcttttgtgtgtctttaacatttatttcagcagagcccaaacattcacacaagaataaagctctgaagtttc
tggcacgtaagatacgcccaagactattcctagacaccagcaaacagacgaggttgaagaagaaacaggtgaaga
ataatgtcttaaacatcattttagcaccctattctgccattctgtatctttataggatatgtccagtccagtccc
agaaaatctactgcccaaggagttctagatgacaggttagatcaagccccggcaaatgtttgcgaaaacaacatt
caactgcctctggacattgcaaagacatattccagggagctatctcttgggctcttttttaacatctctcctaga
ggatgagactcctttcgggattacagccactgggaaagtaaaagcaacagtagcatcaacaggttgcatcgcact
cccgcgccccaggggcggtgcaggagagcgcccggagccagagccagggcgcgctgggcgcaagctggggcgcgc
ccagggccaatgagcttctgattggctctttgctgtgaatagactaggccgaggctaataggaccggagggggcg
ttcacctgggaattggcctcccccccttgccaaggacctccctgattgtatggggcggagtggggtgggggtggg
ggctggaaaggggaagcccaccctaactaaacggaggggcgggaggaggttaaactgaggaagccacgagcccgc
agtaaagagagggcgatcgaggcaaaagggtaggtaacttgggacttctgtgccttgaaagtgttaaggattgca
gggaatgcaggcgcctttcattttactttgggcaccctcccctggctgggcatttaatgaggagtcctactgtgt
gtgttgaaactctgcataataccgtaaaatattctggccctaaattacactcaacggagagatttaggcagatta
caatcctaattggccgcagggtgctggggaaaggggacagagagaattgggggatctggaatttggtgtgctatc
actacaccgcagctctattttccattaagaaaaagatcatatgacgaagtcgaactaagagatctgaagtaaaaa
atgaaaagatggagcaaaagtaagaaacatactctgagacgagtgggttttccccctttattccaacagcaatct
taaatgatggacgtcatgtagcagttatatatctatgaacatgcatgagagatttataaatacctgcatacataa
atacaaacatcctattatacatgagaaatcgtaaatgcttgggcatcagaagtgggagctgtgatcctagcttgg
gggcagcacagggtaggcggccttctctctgctttgagtggcttctgggcgcctggcgggtccagaatcgcccag
agccgcccgcggtcgtgcacatctgacccgagtcagcttgggcaccagccgagagccggctccgcaccgctcccg
caccccagccgccggggtggtgacacacaccggagtcgaattacagccctgcaattaacatatgaatctgacgaa
tttaaaagaaggaaaaaaaaaaaaaaacctgagcaggcttgggagtcctctgcacacaagaacttttctcggggt
gtaaaaactctttgattggctgctcgcacgcgcctgcccgcgccctccattggctgagaagacacgcgaccggcg
cgaggagggggttgggagaggagcggggggagactgagtggcgcgtgccgctttttaaaggggcgcagcgccttc
agcaacc
ATHO1 Promoter 2 (SEQ ID NO: 22):
TCCAAGGTCCGGCAATGAAGTTTGCATAACAAACGTTTGGCAGCTCCCTC TCTCACACCCCATTAACAAGCTGT
AACATATAGCTGCAGGTTGCTATAAT_CTCATTAATATTTTGGAAACTTGAATATTGAGTATTTCTGAGCGCTCA
TT CCCCATATGCCAGACCACTCCTGCCATGCTGACTGGTTCCTTTCTCTCCATTATTAGCAATTAGCTTCTACC
TTCCAAAGTCAGATCCAAGTATCTAAGA TACTACCAAAGGCATCAACTATGTATGCAAGTTAGGCATGCTTAAT
ATCA CCCAAACAAACAAAGAGTCAGCACTTCTTAAAGTAATGAAGATAGATAAA TCGGGTTAGTTCTTTGGGA
CACCGCTGTTGTTTTCCAGAGTTTTTCTATA CTTTAAGCAGCTTGTTTTATATTCTGTCTTTGCCCTCAGCCAG
CTAACAT TTTATTTGTTGAGGGTTTTGGCTCACCACACTTTTGGAAACTTATTTGAT TTCACGGGGAGCTGAA
GGAAGATTGTTTTTGGCAACAGGCAAGTTTAACA CGTTCTTCATGGGGCATTGCGAATGGCACATCTACCAGAA
AGGGAGGGGG AGTAACTTCCTCGTGCTGAACCAGCAGGAGACCAGAGCTTTCCTGAGGTC TTCCTATTGATTT
TAAAGATTTAAAACTGAGCCCCAAAGTTGTAATGTTA TTGAAGTTTGTCTTGGAATATACATCTCCTCTGCTAA
CTTAAAAGTTCAA GAAAGGAAAGGAAAGAAATAGAACCCCTTGCTAACTACAACCTAGACTGA GAGGTGAAGA
TCGCGGGCAAAGACAGGTGGTCACTGAAACGTTTGCAGTT CTTTTCTTCCGAAGGCTTAGGACACAGGGTAAGG
AGGAGCTAAAATAAAG CCGAGTGTACGTTTAGTCTTCTCTGCACCCCAGGCCTAGTGTCTCCCCAG GCAAGGA
GTCACCCCCTTTGCTTCTGGCTCCTAACTGAAAAAGGCAAAAG GGAGTGGAGAATGGGTTAAATCCCAGGACAC
AGGGGAGAGGCAGGGGAGG AGAGAAGTCGGAGGAAGATAAAGGAAAGGACAGGAACCAAGAAGCGTGGG GGTA
GTTTGCCGTAATGTGAGTGTTTCTTAATTAGAGAGCGGCTGACAAT AGAGGGGCTGGCAGAGGCTCCTGGCCCC
GGTGCGGAGCGTCTGGAGCGGA GCACGCGCTGTCAGCTGGTGAGCGCACTCGCTTTCAGGCCGCTCCCCGGG G
AGCTGAGCGGCCACATTTAACACCGTCGTCACCCTCCCCGGCCTCCTCA ACATCGGCCTCCTCCTCGTAGACAG
CCTTGCTCGGCCCCCCACCGGCAGA GTTTACAGAAGCCAGAGCCTCTCGCCGTTCCCCCGCATTCGCCCGGG

In one embodiment, the promoter or enhancer sequence is a SIX1 promoter or enhancer. A SIX1 promoter or enhancer refers to a regulatory polynucleotide sequence comprising or consisting of a nucleic acid sequence sufficient to direct expression of a downstream polynucleotide in an outer or inner hair cell, a vestibular hair cell, a spiral ganglion, or a vestibular ganglion and having at least about 85% sequencing identity to the following sequence or the nucleotide sequence of GH14J060640. Examples may comprise or consist of the corresponding sequence or a functional fragment thereof.

SIX1 Promoter
(SEQ ID NO: 23)
ggcaattggggaggaggaaggcattgggagaaaacaacccgaaggaaaa
agaagctggctctttgcaatgggagaaggggttcactcctgaactaggg
gcaggcctgcgcctgggcggacgctgtagggtgggaacgctcgataact
ggagagtcccagagaccaccaggcctttccgcaccaggaaaaaaaaaaa
aaatctgctctgagaatcttcatctcggtccaccagggtgagtttgggc
ctggaaaatgggggaaagaaaagagagtagcgaaaggttttcgggttgc
ggcgccccgcgtgcagacgtctgctctccccagccgcccagcctgctgt
ggcagccacgctgtgcgcgtgtctttatccttcagttcaaaccagcccc
cacatcttgaagtaatcagcagccgagaagagacttgtccccctggcat
ctcaggttaaagaaggaggtccaggaaaatgtggtagagccattacaag
aaacccgagccgcagtcatcagggactccaattgccatcgcattaggat
tgattagagaccagcagtaccgtaatgacccggaaagaggggcagccgg
ggattattaatatcagaatgcgggggtggggggggagtgggtgggcaaa
gtgggtagggagaagtttcaaattactaggatttatatgtaagtgcagg
tttacctgaggaaaccagatagtcagtgaatcacgctaacgataaatct
aattgcatttcaaatttcacgaagcctgatttttttggagagtggagta
aaaccatcatgaaaccaagccaatatcttagtttttataaacacatagt
tttataagcttattgtaagaattcaagtgaagagatggggaggggtgct
aaatgtctgctttccttgtcactgggcctccttaccatttggactccaa
atagcttgatttttcttctcgtgggagtacatttttgagtcgctttggt
tcttccctccagggatatggggcaatggagtggacattttttcagagcc
tgcagaggagtaataaaccaaaatttaagtgaagcagtgatacctgggt
agtggtttttgttttttgttttgttgaagacatgaatgatcaggatccc
aaggagatgtggagaagagtggtttgtctccttcctccaaatatgagcc
aaaacaaatcagcccctgatggtctgggctgaaatgcagctcactggat
aattaactccatctttccagtccaccctaactgtttatttagtgtcacc
ctgcattaccgtcactagcactttcctctgaacaggaggagctgcaggc
tttcacctcccaaaaggaaattctttttagcttagacagatgtggtatt
caggatgtacataagttgagaaaaaaaagttgattggaaattcaatgga
agatagcggttttggagagtttcatttcataaacctgccctaaggagac
attttaaaaaacagggaatggggggtggggcgcagagtccctttaattc
ttaagaattgtttcttgtgacaccttggctccactttccagagaggtag
ttggcatctgatgttgagtagggtcttctagctcaaacctggcgattgg
tcagagctttctcagagggcaatttgttaacccttttctcccctaggtg
gccaatacacctttgcccgccctccgtgcaggctacaccatctattgcc
cgccctccactccctgtgaaaggttctcgccaagtaaggcctactacac
cgcccagacttgagggtctactttcaagagaacttacctgcgctgcctc
agtgggtgacataagcacactctgtgaaacctcacagcgcccagtcccg
gagagaaaaaaacgagcgcccagagccgaggggaagggagagggaggga
aggaggaagaaagaggaggatgagaagaggaagaggaaaaacaatgaga
aagaagagagagagataaggagagagaatgagaaacaggttcacataca
gacagaatgaacgagcgtcctctttcagatggagaaaagagttagagac
acactctgggaaagacctgccacggggacacacgtagagggagagagag
aataactcttccaaagacacacaccccagctctgttcccctccagtctg
agctccctcccagtgcttgaccctcagtcggtccatcagtctgtgtgtg
tttctctctttctcccttccctgacctcgagacaatacggagatggtat
tttcgggagccgcgcggggcggcagggttttcagcggcgagccaggctc
acagctggctctgtttgtccaggtgtgggcggtggaggtggtggggggc
gtcagtcgccctgaatcttgggccagcgcagcgttttcgcaccaaagaa
gtaggaggctctgctctcgagcttcccgcttccaggagaaaacttcctc
cctccatacatacataaatacacttggggaagagacaaatcttcattgt
tcagaaataagctgaggaggcccgcctgctcccctcagccaggcagagc
gaggcagggagggaggcgggggccgcgcggatccggtgtcacgttgcag
attcccactaaagataacgaaacgtgatctatttttcagaacagcaagt
ggatgggaaagaaccttgacaacacaaaactcatttactaatggactgg
ttttgggtttctcttctctgagtccgcaaatctctctgcctgagcccca
ggcctattcagacctcgcggggagcggcgtcccccaggagcaggtgggc
tgctgctccggcgccacgccggccatcggagccggagttcccaagggtc
gagggcacccggagtttccgactaagcagaggaaaaagaattttggcac
ttcagccggatttggggagtctccgcactgccccagagcgatgacacga
atttattgaagaaacacctctctatgccctccctttccaaatatttgta
ggtagagtagaatcctcaccgtcgttctggatggtgaaagaattccaag
aaagcggcccgggagcccctaactatcttgctacaagccagaagggaaa
caagtgggtcccagctaggggtgtttgcatcaaggcctgtcaacccagc
cgagaaaccctagcaacagctaatgctgtcccactgaatgctttttcat
cgactaaagcgcacgcggccgttacctgcacagaggaggcactggagag
ccgaaacctggcatcgggcgctccttgccagcggtgttccctcccgagc
cgcgctgcagcgagggcagcagccggctggctagagttgctcatttcct
ttaaatctccaacgcatctctgcttttcgtgcgatttgcattgacagca
aaggttttatgttacgagaacaactcaaacctttctgtgtgcgatttaa
aatggatcatttagtttaggtggtttgtaaattcatgtcattttacctc
tttcagcggttttcagtgaaaggttccattttgggaaaccttatatatc
cgagccaataattaacatttataacaattggttcccctaaccgagggat
cgaaatagcaggtagactccttggtataaagcgcatagcactttccctt
tccagttacgcgatctattggattaagcgggaggaaaaatgttggccgg
cccagtcacggcctccgaggaagagcacctcttttctgcccgggttccc
gcaatcacaccttctacacgtcgtcttcccagatgacggcaatgtagta
cgtcctggttccttttgaagacagataaaaagaaatgggcaggtaggtc
tggaggaagccgggaaaaaaaggtggtcctatttgtgtctaattgccat
ctctgctgtcatcctctgtaaatttatacctgcccttatttgtgcaggc
tatgcctcatttccttatacaaaattgttaattacaaagcacaccccat
actcaacatactgtttgtcttttaagagacgtggttgtgcttaaagacc
ctccccccattgtaattgtgcgcacttgctccagactttagggtattaa
ctgatttttagagccgggtttaagcctccgttccggtcctcacacagct
gccatgtcaataaggactgtttcaagcagaggcattccttctgggaggg
cactgcagctccttaagacttcatgtgaaacatcataagcactaaaata
agacagtgaggggaaattaccagacatggctctgggtttagcagtgcca
ggcttccagtttagaaagtcattgggcatactatatggcagaggctctt
ttgaaagcatgcacacctattaaatgcatttttttaaaaaaaaagccta
aggtaatgaaatatcagcatctgcttccaagatctaattgcatggaata
agcaagagtttcaaggatgcagaaaacattatgttcagcagagaagcag
cttcctgtctacttggaatctcccatgtggcagccctgaggtgggaacc
ctttatgagtttagaatgtatgttgcagggtgcactgtactgggtgcat
cattttaaaaccatattaataataaaagaggaaaccaagacatgcttct
ccttctcccacttgcatctcaatatgcaaaaggttctgtagtgctgggc
tctttggctaaaggaatcaaaacatatttgaagaagacaagccagttta
aggagcctacagtttgttcttcaccactcactaagagggcacattgaaa
tcaaaagcatttttgaagtgtacttagaattctgttttgaacttatcta
atcagagataagtatagtcataaagtcactctccttccaccatttggct
tcccttatctccacgcattgttcttatctcctgctagtctcagctactg
acaatagattatctttgtttagagaggctaattgttttcaaaatccttt
taattattgctttggaactaggtgtgcaaatgaggcttaagataattac
aacttcacttgtttgtgcagttttactaagcattctccacacataaaca
aaacagcagatgatgggacttaaattgcagtgcatctctcattccctgg
cagcaaaataggccagcattgcaacacacacacacacacacatacacac
acacacacacacacacacacctctttatttaatcttcttggagccgctg
tcgttctcgctttgagggcagtgtccctctcagagcagggtaaggatta
tattacaaccggacggttattggagccgggcacagttttatttaattta
caccacatccagcgcctttcgtacgaggttcccaaagcatttttgctaa
tagcaagtaaagcctctcggcattcctcagtagcagaggtggcactgca
aaacacaaccgcacagatggggaaattgagtccagaagcttcaggaacc
tgctggtttcaatcgcagaacattaagataccaaatcacaaatgaacgc
tcggagtctctaggtgctcagcaagtatgctctaaacagctctctgcct
ggtctcaaatcatggaggtcttttgtgaacgagggaaacgctgtccgac
tgggagtacttgtgccctttggagggtggtgtgcagcagtggggggagg
acagtggtagttggtggggggagggaggaggaaatagggttggactcct
cgagctgaagccaacccgaagccactcttccagaggctgcatgcgcgga
ccctggtctgctcttgaattatttatcgcgttccataagctattcatca
atccattatttattcattccaacattaaagtaattaaatatgagctcac
cgggagagggcaagtctgcaagggatgaggacaagagtacggagggaga
ggggaggaattaaggtttggagtcattgatttgtgcggagttgtgcgcg
actccccttaggtgatactggcgacgggtcccgcgttgcagggtcctga
cgcgctcacccactccctacctccccacacctccgcagcgcagctccct
cagaccccaactggcttcccacctttcctcttccttctcctcccaccac
tccctacccgcgcagccccggcaacacccccaacccccagctccgcagc
ctcgccctccccgctctgggcggtgcggcctctctgcccagccgggcgg
cgcgctggctggccagccccggataggcgccgccggcagccaatcagcg
tgccggggacgctgcgcgctttaaggccgctgcggggagaacagaaacc

In one embodiment, the promoter or enhancer sequence is a SOX21 promoter or enhancer. A SOX21 promoter or enhancer refers to a regulatory polynucleotide sequence comprising or consisting of a nucleic acid sequence sufficient to direct expression of a downstream polynucleotide in an outer or inner hair cell, a vestibular hair cell, a spiral ganglion, or a vestibular ganglion and having at least about 85% sequencing identity to the following sequence or the nucleotide sequence of GeneHancer (GH) Identifier GH13J094705. Examples may comprise or consist of the corresponding sequence or a functional fragment thereof.

SOX21 Promoter
(SEQ ID NO: 24)
ataaatcgtaggaagacttaaagagttaaccgtttgtagatgttcctac
ttgcacataccacagggctaggtcaaatccaaactgccaaagaaagaag
gcaagcaagttctccctgaagggcaggccaccatgtgaataaaacctcc
agacagatcatgttctaaatgtgccgcagcataccaaaaagtgaggaaa
atacggaaacagaatcatgctgtttgacttctgcatcgtgttaactatt
ttttttaaaaaactgctctaactactaaagaaggaaattatgcttagca
taaaaatattccagcactgagtgattggtacgttttacttctctcttcc
agggaaactacttacacttattgattttaaacaccttcacctcagcagc
gcatgtaagtgagacactgtaagttaactgtaccttaaagagatcagta
tatagatggttctggcgaccaagattacaacaaaagacaaacaacacag
aatttttaaagagctgaatagtcagtcaaggatgcatactctcagtctg
ggctagctcttttttggaaaggttgccaagaatgtccttattccctcca
gagttatttttcactccactacctctgactaggtgcatcgccagtgcaa
gaaaatgcgagaagcctctgtttctgttgcaagcaacacctggacctcc
aaggaaaatgagcactttcttgctaggaacagtctcaaacattttcaag
ggtgagtctccccttctccaggtttaaccctcttcctccgtagctggag
aggctctcctactcgtgcccgccgccttcttcctcccggcgaggattcc
agggccacaaccagggcagctgcacgccaggccggcctgggtagccggg
atgacaccagggtttgccaacgccaaacagggcgcagactccccggcgc
gctccggactctccactcgcctaaacctgctctcaagtgaaaaggagtg
ggaggaggaggattgggagggggcggggggcggaggggatctgttaatt
cgaggtcctgggggtgtgggagcgggaggcggctccgctgatccctcct
ccctccccgagtcctgggctccacctcctccgcaggcccccagatatac
acacacagccgcggctggacatgcacacccagtgccggctccgggcgcg
cacacatgcaggtagccacactcacctgagccggtgcagagggcggcct
gggctgggaaggggccccctgcgccgcgggagtggggagagcgggaggt
gccacagcgcggacctgcgaactgaggtgatggcccgttttccttaact
ctcctgcagcacctccgcccctgcgccggctcggctctggccacagcag
cgaggacagaggggccccgggcgcggcggactcgcgggcgcacgctccc
aggagcgcgccgagaggggcgccgcggcgcaggaaacttgggcgaagtt
agttgcaagtgccaggcggctgccgagaagagcgaggcggggacgcggc
cggtgccaggcctgcggcgccgcgtggaggctgcgctgccctccgcggc
cggcgagctccgggagccctaagtcggcaggcgcgcctgaaggggctcg
gagcgaacagagccggcatggcccaggggacggccggacgcggctcctc
ggcgagccgggagaacttcctctgaggccgggcgaccaacgcttgctca
agcctcatccaccctccttcctcgccccacccgtcagtccagcagcggg
agaggaagactcgagaggcaggtgctgcgggggacagctcccccctcca
aaaaaaaaaaatcacccagccgattgggggtttcccatcggcgcaccct
gcccggagccaagaagacaggctggtgctgctgtatttgtatttatatc
cattgctgcgctctgcgttctcgtggcacgcctggacactcctccgcct
ccccctcctcttcctcctccagggccacctccccgccttccccaccccc
atctgcttctgtcaaatgagaaagtcaccgaggagaaccc

### Viral Vectors

Any convenient viruses may be utilized in delivering the vector of interest to the subject. Viruses of interest include, but are not limited to a retrovirus, an adenovirus, an adeno-associated virus (AAV), a herpes simplex virus and a lentivirus. Viral gene therapy vectors are well known in the art, see e.g., Heilbronn & Weger (2010) Handb Exp Pharmacal. 197:143-70. Vectors of interest include integrative and non-integrative vectors such as those based on retroviruses, adenoviruses (AdV), adeno-associated viruses (AAV), lentiviruses, pox viruses, alphaviruses, and herpes viruses.

In some cases, non-integrative viral vectors, such as AAV, may be utilized. In one aspect, non-integrative vectors do not cause any permanent genetic modification. The vectors may be targeted to adult tissues to avoid having the subjects under the effect of constitutive expression from early stages of development. In some instances, non-integrative vectors effectively incorporate a safety mechanism to avoid over-proliferation of a transgene-expressing cells. The cells may lose the vector (and, as a consequence, the protein expression) if they start proliferating quickly.

Non-integrative vectors of interest include those based on adenoviruses (AdV) such as gutless adenoviruses, adeno-associated viruses (AAV), integrase deficient lentiviruses, pox viruses, alphaviruses, and herpes viruses. In certain embodiments, the non-integrative vector used is an adeno-associated virus-based non-integrative vector, similar to natural adeno-associated virus particles. Examples of adeno-associated virus-based non integrative vectors include vectors based on any AAV serotype, i.e., AAVI, AAV2, AAV3, AAV4, AAV5, AAV6, AAV7, AAV8, AAV9, AAVIO, AAVII and pseudotyped AAV. Vectors of interest include those capable of transducing a broad range of tissues at high efficiency, with poor immunogenicity and an excellent safety profile. In some cases, the vectors transduce post-mitotic cells and can sustain long-term gene expression (up to several years) both in small and large animal models of the related disorders.

### Pharmaceutical Compositions

The polynucleotides or vectors described herein may be incorporated into a vehicle for administration into a patient, such as a human patient suffering from sensorineural hearing loss and/or vestibular dysfunction. Pharmaceutical compositions containing vectors, such as viral vectors, that contain a polynucleotide described herein operably linked to a therapeutic transgene can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing a polynucleotide described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intracisternal, intratheca, intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl.sub.2), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

Pharmaceutical compositions are typically formulated as liquids, semisolids (e.g., gels) or solids (e.g., matrices, scaffolds and the like, as appropriate. Liquid compositions can be formulated for administration by any acceptable route known in the art to achieve delivery of agent, compound, cell, or composition to the target tissues (e.g., inner ear). Typically, these include injection or infusion into the CNS or PNS, either in a diffuse fashion or targeted to the site of disease or distress, by a route of administration including, but not limited to, intraocular, intracerebral, intraventricular, intracerebroventricular, intrathecal, intracisternal, intraspinal and/or peri-spinal routes of administration by delivery via intracranial or intravertebral needles and/or catheters with or without pump devices. When injected or infused, the agent, compound, cell, or composition may migrate to the site of interest.

### Methods of Treatment

In some embodiments, the present disclosure provides novel therapeutic strategies for treating hearing loss or balance loss associated with a damage or loss of inner ear cells, e.g., cochlear hair cells or vestibular hair cells, respectively

The compositions described herein may be administered to a subject with sensorineural hearing loss and/or vestibular dysfunction by a variety of routes, such as local administration to the inner ear (e.g., administration into the perilymph or endolymph, e.g., intracisternal, intratheca, through the oval window, round window, or a semicircular canal (e.g., the horizontal canal), e.g., administration to a cochlear or vestibular hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patients age, body weight, sex, severity of the disease being treated, the patient's diet, and the patient's excretion rate. Compositions may be administered once, or more than once (e.g., once annually, twice annually, three times annually, bi-monthly, or monthly).

Subjects that may be treated as described herein are subjects having or at risk of developing sensorineural hearing loss and/or vestibular dysfunction (e.g., subjects having or at risk of developing hearing loss, vestibular dysfunction, or both). The compositions and methods described herein can be used to treat subjects having or at risk of developing damage to cochlear hair cells (e.g., damage related to acoustic trauma, disease or infection, head trauma, ototoxic drugs, or aging), subjects having or at risk of developing damage to vestibular hair cells (e.g., damage related to disease or infection, head trauma, ototoxic drugs, or aging), subjects having or at risk of developing sensorineural hearing loss, deafness, or auditory neuropathy, subjects having or at risk of developing vestibular dysfunction (e.g., dizziness, vertigo, or imbalance), subjects having tinnitus (e.g., tinnitus alone, or tinnitus that is associated with sensorineural hearing loss or vestibular dysfunction), subjects having a genetic mutation associated with hearing loss and/or vestibular dysfunction, or subjects with a family history of hereditary hearing loss, deafness, auditory neuropathy, tinnitus, or vestibular dysfunction. In some embodiments, the subject has hearing loss and/or vestibular dysfunction that is associated with or results from loss of hair cells (e.g., cochlear or vestibular hair cells). The methods described herein may include a step of screening a subject for mutations in genes known to be associated with hearing loss or vestibular dysfunction prior to treatment with or administration of the compositions described herein. A subject can be screened for a genetic mutation using standard methods known to those of skill in the art (e.g., genetic testing). The methods described herein may also include a step of assessing hearing and/or vestibular function in a subject prior to treatment with or administration of the compositions described herein. Hearing can be assessed using standard tests, such as audiometry, auditory brainstem response (ABR), electrocochleography (ECOG), and otoacoustic emissions. Vestibular function may be assessed using standard tests, such as eye movement testing (e.g., electronystagmogram (ENG) or videonystagmogram (VNG)), posturography, rotary-chair testing, ECOG, vestibular evoked myogenic potentials (VEMP), and specialized clinical balance tests, such as those described in Mancini and Horak, Eur J Phys Rehabil Med, 46:239 (2010). The compositions and methods described herein may also be administered as a preventative treatment to patients at risk of developing hearing loss and/or vestibular dysfunction, e.g., patients who have a family history of hearing loss or vestibular dysfunction (e.g., inherited hearing loss or vestibular dysfunction), patients carrying a genetic mutation associated with hearing loss or vestibular dysfunction who do not yet exhibit hearing impairment or vestibular dysfunction or patients exposed to risk factors for acquired hearing loss (e.g., disease or infection, head trauma, ototoxic drugs, or aging) or vestibular dysfunction (e.g., acoustic trauma, disease or infection, head trauma, ototoxic drugs, or aging).

The compositions and methods described herein can be used to promote or induce hair cell regeneration in a subject (e.g., cochlear and/or vestibular hair cell regeneration). Subjects that may benefit from compositions that promote or induce hair cell regeneration include subjects suffering from hearing loss or vestibular dysfunction as a result of loss of hair cells (e.g., loss of hair cells related to trauma (e.g., acoustic trauma or head trauma), disease or infection, ototoxic drugs, or aging), and subjects with abnormal hair cells (e.g., hair cells that do not function properly when compared to normal hair cells), damaged hair cells (e.g., hair cell damage related to trauma (e.g., acoustic trauma or head trauma), disease or infection, ototoxic drugs, or aging), or reduced hair cell numbers due to genetic mutations or congenital abnormalities. The compositions and methods described herein can also be used to promote or increase hair cell survival (e.g., increase survival of damaged hair cells, promote repair of damaged hair cells, or preserve hair cells in a subject at risk of loss of hair cells (e.g., loss of hair cells due to age, exposure to loud noise, disease or infection, head trauma or ototoxic drugs)).

The compositions and methods described herein can also be used to prevent or reduce ototoxic drug-induced hair cell damage or death (e.g., cochlear and/or vestibular hair cell damage or death) in subjects who have been treated with ototoxic drugs, or who are currently undergoing or soon to begin treatment with ototoxic drugs. Ototoxic drugs are toxic to the cells of the inner ear, and can cause sensorineural hearing loss, vestibular dysfunction (e.g., vertigo, dizziness, or imbalance), tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods described herein prevent or reduce hair cell damage or death related to acoustic trauma, disease or infection, head trauma, or aging.

The transgene operably linked to a promoter or a functional portion or derivative thereof for treatment of a subject as described herein can be a transgene that encodes a protein expressed in healthy hair cells (e.g., cochlear and/or vestibular hair cells, e.g., a protein that plays a role in hair cell development, function, cell fate specification, regeneration, survival, or maintenance, or a protein that is deficient in a subject with sensorineural hearing loss and/or vestibular dysfunction) or another therapeutic protein of interest. The transgene may be selected based on the cause of the subject's hearing loss or vestibular dysfunction (e.g., if the subject's hearing loss or vestibular dysfunction is associated with a particular genetic mutation, the transgene can be a wild-type form of the gene that is mutated in the subject, or if the subject has hearing loss associated with loss of hair cells, the transgene can encode a protein that promotes hair cell regeneration), the severity of the subject's hearing loss or vestibular dysfunction, the health of the subject's hair cells, the subject's age, the subject's family history of hearing loss or vestibular dysfunction, or other factors. The proteins that may be expressed by a transgene operably linked to a promoter described herein for treatment of a subject as described herein include VGLUT3, MYO7A (USH1B), USH1C, CDH23, PCDH15 (USH1F), SANS (USH1G), USH2A, ADGRV1/VLGR1, WHRN (DFNB31), USH3A (CLRN1), HARS, Cx26 (GJB2), Cx30 (GJB6), Cx29 (GJC3), Cx31 (GJB3), ACTG1, FSCN2, RDX, POU4F3, TRIOBP, TPRN, XIRP2, ATOH1, GFI1, CHRNA9, CIB3, CDH23, PCDH15, KNCN, DFNB59, OTOF, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, CABP2, EPS8, EPS8L2, ESPN, ESPNL, PRPH2, STRC, SLC8A2, ZCCHC12, LRTOMT2, LRTOMT1, USH1C, ELFN1, TTC24, DYTN, KCP, CCER2, LRTM2, KCNA10, NTF3, CLRN1, CLRN2, SKOR1, TCTEX1 D1, FCRLB, SLC17A8, GRXCR2, BDNF, SERPINE3, NHLH1, HSP70, HSP90, ATF6, PERK, IRE1, BIP, GJB2, and USH1G.

Treatment may include administration of a composition containing the nucleic acid vectors (e.g., AAV viral vectors) containing a promoter described herein in various unit doses. Each unit dose will ordinarily contain a predetermined-quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the cochlea). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the viral vectors may be administered to the patient at a dose of, for example, from about 1×10¹⁰ vector genomes (VG) to 1×10¹⁵ VG in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL).

The compositions described herein are administered in an amount sufficient to improve hearing, improve vestibular function (e.g., improve balance or reduce dizziness or vertigo), reduce tinnitus, increase expression of a therapeutic protein encoded by a transgene, increase function of a therapeutic protein encoded by a transgene, prevent or reduce hair cell damage, prevent or reduce hair cell death (e.g., ototoxic drug-induced hair cell death, age-related hair cell death, or noise (e.g., acoustic trauma)-related hair cell death), promote or increase hair cell development, increase hair cell numbers (e.g., promote or induce hair cell regeneration), increase or promote hair cell survival, or improve hair cell function. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. Vestibular function may be evaluated using standard tests for balance and vertigo (e.g., eye movement testing (e.g., ENG or VNG), posturography, rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss and/or vestibular dysfunction (e.g., in subjects who carry a genetic mutation associated with hearing loss or vestibular dysfunction, who have a family history of hearing loss or vestibular dysfunction (e.g., hereditary hearing loss or vestibular dysfunction), or who have been exposed to risk factors associated with hearing loss or vestibular dysfunction (e.g., ototoxic drugs, head trauma, acoustic trauma, or infection) but do not exhibit hearing impairment or vestibular dysfunction (e.g., vertigo, dizziness, or imbalance), or in subjects exhibiting mild to moderate hearing loss or vestibular dysfunction). Expression of the therapeutic protein encoded by the transgene operably linked to a promoter described herein in the nucleic acid vector administered to the subject may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of the compositions described herein. Hair cell numbers, hair cell function, or function of the therapeutic protein encoded by the nucleic acid vector administered to the subject may be evaluated indirectly based on hearing tests or tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to hair cell numbers, hair cell function, or function of the therapeutic protein prior to administration of the compositions described herein. Hair cell damage or death may be reduced by 5% or more (e.g., 5%, 10%, 15%, 20%, 30%, 40%, 50%, 60%, 70%, 80%, 90%, 100%, 125%, 150%, 200% or more) compared to hair cell damage and death typically observed in untreated subjects. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating sensorineural hearing loss or vestibular dysfunction. Compositions may include a polynucleotide described herein or a nucleic acid vector containing such polynucleotides. The nucleic acid vectors may be packaged in an AAV virus capsid (e.g., AAV1, AAV2, AAV2quad(Y-F), AAV6, AAV9, Anc80, Anc80L65, DJ/9, 7m8, or PHP.B). The kit can further include a package insert that instructs a user of the kit, such as a physician, to perform the methods described herein. The kit may optionally include a syringe or other device for administering the composition.

### Definitions

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. A “nucleic acid fragment” is a fraction of a given nucleic acid molecule. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid”, “nucleic acid molecule”, “nucleic acid fragment”, “nucleic acid sequence or segment”, or “polynucleotide” may also be used interchangeably with gene, cDNA, DNA and RNA encoded by a gene.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments are well known in the art (see, e.g., Nelson, MAbs (2010) 2(1): 77-83) and include but are not limited to Fab, Fab′, Fab′-SH, F(ab′)₂, and Fv; diabodies; linear antibodies; single-chain antibody molecules including but not limited to single-chain variable fragments (scFv), fusions of light and/or heavy-chain antigen-binding domains with or without a linker (and optionally in tandem); and monospecific or multispecific antigen-binding molecules formed from antibody fragments (including, but not limited to multispecific antibodies constructed from multiple variable domains which lack Fc regions).

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

A “therapeutic RNA molecule” or “functional RNA molecule” as used herein can be an antisense nucleic acid, a ribozyme (e.g., as described in U.S. Pat. No. 5,877,022), an RNA that effects spliceosome-mediated trans-splicing (see, Puttaraju et al. (1999) Nature Biotech. 17:246; U.S. Pat. Nos. 6,013,487; 6,083,702), an interfering RNA (RNAi) including siRNA, shRNA or miRNA, which mediate gene silencing (see, Sharp et al., (2000) Science 287:2431), and any other non-translated RNA, such as a “guide” RNA and CRISPR RNA (Gorman et al. (1998) Proc. Nat. Acad. Sci. USA 95:4929; U.S. Pat. No. 5,869,248) and the like as are known in the art.

“Anti-sense” refers to a nucleic acid sequence, regardless of length, that is complementary to the coding strand or mRNA of a nucleic acid sequence. Antisense RNA can be introduced to an individual cell, tissue or organanoid. An anti-sense nucleic acid can contain a modified backbone, for example, phosphorothioate, phosphorodithioate, or other modified backbones known in the art, or may contain non-natural internucleoside linkages.

As referred to herein, a “complementary nucleic acid sequence” is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

As used herein, the term “siRNA” intends a double-stranded RNA molecule that interferes with the expression of a specific gene or genes post-transcription. In some embodiments, the siRNA functions to interfere with or inhibit gene expression using the RNA interference pathway. Similar interfering or inhibiting effects may be achieved with one or more of short hairpin RNA (shRNA), microRNA (mRNA) and/or nucleic acids (such as siRNA, shRNA, or miRNA) comprising one or more modified nucleic acid residue—e.g., peptide nucleic acids (PNA), locked nucleic acids (LNA), unlocked nucleic acids (UNA), or triazole-linked DNA. Optimally, a siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

An “imaging agent” is a compound that has one or more properties that permit its presence and/or location to be detected directly or indirectly. Examples of such imaging agents include proteins and small molecule compounds incorporating a labeled moiety that permits detection. An imaging agent can be any chemical or substance that is used to provide the signal or contrast in imaging. Examples include an organic molecule, metal ion, salt or chelate, particle, labeled peptide, protein, polymer or liposome.

“Subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like. “Mammal” refers to any mammal including but not limited to human, mouse, rat, sheep, monkey, goat, rabbit, hamster, horse, cow or pig.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

“Supporting Cell” as used herein in connection with a cochlear epithelium comprises epithelial cells within the organ of Corti that are not hair cells. This includes inner pillar cells, outer pillar cells, inner phalangeal cells, Deiter cells, Hensen cells, Boettcher cells, and/or Claudius cells.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “hair cell-specific expression” refers to production of an RNA transcript or polypeptide primarily within hair cells (e.g., cochlear hair cells and/or vestibular hair cells) as compared to other cell types of the inner ear (e.g., spiral ganglion neurons, glia, or other inner ear cell types). Hair cell-specific expression of a transgene can be confirmed by comparing transgene expression (e.g., RNA or protein expression) between various cell types of the inner ear (e.g., hair cells vs. non-hair cells) using any standard technique (e.g., quantitative RT PCR, immunohistochemistry, Western Blot analysis, or measurement of the fluorescence of a reporter (e.g., GFP) operably linked to a promoter). A hair cell-specific promoter induces expression (e.g., RNA or protein expression) of a transgene to which it is operably linked that is at least 50% greater (e.g., 50%, 75%, 100%, 125%, 150%, 175%, 200% greater or more) in hair cells compared to at least 3 (e.g., 3, 4, 5, 6, 7, 8, 9, 10, or more) of the following inner ear cell types: Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human hair cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human hair cell).

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule that can be joined to a second molecule, wherein the molecules are so arranged that the first molecule affects the function of the second molecule. The term “operably linked” includes the juxtaposition of two or more components (e.g., a promoter and another sequence element) such that both components function normally and allow for the possibility that at least one of the components can mediate a function that is exerted upon at least one of the other components. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. In additional embodiments, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Lorence, Recombinant Gene Expression: Reviews and Protocols (Humana Press, New York, N.Y., 2012).

As used herein, the term “promoter” refers to a recognition site on DNA that is bound by an RNA polymerase. The polymerase drives transcription of the transgene.

By “transgene” is meant any piece of DNA that is inserted by artifice into a cell and becomes part of the genome of the organism that develops from that cell. Such a transgene may include a gene which is partly or entirely heterologous (i.e., foreign) to the transgenic organism, or may represent a gene homologous to an endogenous gene of the organism.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “administer” or “administration” refers to a method of delivering an agent, a compound, a cell, or a compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. The agent, compound, cell, or compositions may be formulated for administration by any acceptable route known in the art to achieve delivery of drugs and biological agent to the target tissue (e.g., inner ear), including, but not limited to, oral, nasal, ophthalmic and parenteral, including intravenous. Particular routes of parenteral administration include, but are not limited to, intramuscular, subcutaneous, intraperitoneal, intracerebral, intraventricular, intracerebroventricular, intrathecal, intracisternal, intraspinal and/or peri-spinal routes of administration by delivery via intracranial or intravertebral needles and/or catheters with or without pump devices. In one embodiment, the agent, compound, or composition described herein are administered intrathecally and intracisternally. In some embodiments, administration can be auricular, intraauricular, intracochlear, intravestibular, or transtympanically, e.g., by injection. In some embodiments, administration is directly to the inner ear, e.g., injection through the round or oval, otic capsule, or vestibular canals. In some embodiments, administration is directly into the inner ear via a cochlear implant delivery system. In some embodiments, the substance is injected transtympanically to the middle ear. In certain embodiments “causing to be administered” refers to administration of a second component after a first component has already been administered (e.g., at a different time and/or by a different actor).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, agents described herein are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, the term “pharmaceutical composition” refers to the active agent in combination with a pharmaceutically acceptable carrier commonly used in the pharmaceutical industry.

As used herein, the term “vector” includes a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin

As used herein, the terms “virus vector” or “viral vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value.

A “mixture” is intended to include solutions, dispersions, suspensions, solid/liquid mixtures, and liquid/liquid mixtures. Solutions, unlike dispersions, suspensions, and mixtures, lack an identifiable interface between their solubilized molecules and the solvent. Hence, the term mixture may be used when a solid is in direct contact with a liquid (a solution) and when the solid is merely carried or suspended by the liquid. In either instance, the liquid may be referred to as a “solvent.”

The term “fusogenic” describes the ability of a vesicle to fuse with, thus becoming part of, a target cell membrane.

A “fusogen” is any substance that increases the ability of a lipid vesicle bilayer to fuse with, thus becoming part of, a target cell membrane. Upon fusing, the lipid vesicle may release the contents of the vesicle into the interior of the cell. Fusogens exclude stable vesicle formers and may destabilize the vesicle.

### EXAMPLES

### Example 1

This example descibes material and methods used in Examples 2-6 bellow.

### Animals

We used male C57BL/6JRj mice (Janvier Labs, Le Genest Saint-Isle, France), Prox1-EGFP+ mice (Choi et al., 2011), kindly provided by Dr. Kari Alitalo, by crossing with C57BL/6JRj background (Janvier Labs, Le Genest-Saint-Isle, France), Aqp4-knockout and VGLUT3 knockout mice (Vogl et al., 2016), kindly provided by Prof. Dr. Tina Pangrsic Vilfan, Department for Otolaryngology, University Medical Center Gottingen, aging from 4 weeks to 3 months of age. Mice mainly were housed in groups (2-5 mice per cage) with controlled temperature and humidity, on 12 hours light/dark cycles (lights on at 6:00) and fed with regular rodent chow and watered with sterilized tap water ad libitum. All experiments in Denmark were approved by the Animal Experiments Council under the Danish Ministry of Environment and Food (license number: 2015-15-0201-00535 and 2020-15-0201-00480). All procedures in Sweden followed the regulations of Karolinska Institutet and were approved by the regional ethics committee of Stockholm (Stockholm's Norra Djurförsöksetiska Nämnd, 11778-2019). The procedures were performed in accordance with the European directive 2010/63/EU, with due care to minimize the number of animals included in the study.

Injection into Cisterna Magna of Mice

The protocol used in this study to inject tracers and AAVs into cisterna magna (CM) is an adaptation from the chronic cannula implantation procedure previously described (Xavier et al., 2018). Briefly, mice were anesthetized with a mixture of Ketamine/Xylazine (100 mg/kg, 10 mg/kg, respectively), after depth of anesthesia was confirmed by the cessation of reflexes, the incision area was properly shaved and sterilized with the aid of ethanol 70% swabs (Vitrex Medical A/S Herlev, DK), followed by iodine solution (Povidone Iodine 7.5% Henry Schein, Melville, NY, USA) applicated using cotton swabs. Before the onset of surgery, analgesics were applied, Lidocaine (0.05 ml, 0.2 mg/ml) was applied in the incision site and Buprenorphine (0.05 mg/kg) injected subcutaneously for post-surgical analgesia. After performing the skin incision around the occipital crest, the neck muscles were separated to reveal the CM underneath. A 30 G needle (SOPIRA® Carpule 30G 0.3×12 mm, Kulzer, Hanau, Germany) was attached to one extremity of a PE 10 tube filled with ACSF, while the other extremity of the tube is connected to a syringe (Hamilton syringe GASTIGHT®, 1700 series, 1710TLL, volume 100 μL, PTFE Luer lock, Reno, NV, USA), which in turn is placed in a syringe pump (LEGATO® 130 Syringe pump, KD Scientific, Holliston, MA, USA). The pump was used to aspirate injection medium into the tubing and the needle was then inserted into the CM through a puncture into the dural membrane and kept in place with cyanoacrylate glue (Loctite super glue gel control) cured with a drop of glue accelerator (Insta-Set™ CA Accelerator, Bob Smith Industries, Atascadero, CA, USA).

### IV Injections

4 week old animals were placed under a heating lamb, and were awake while injected in the tail veins with 100 μL of 1/10 diluted ssAAV-PHP.B/2-hCMV-chI-EGFP-WPRE-SV40p(A) (4.7*10E13 vg/ml) solution. After the injections the animals were marked and placed in the cage and lived for 14 days before perfusion and tissues were processed as described below.

### Virus

For eGFP virus injections 10 μL of ssAAV-PHP.B/2-hCMV-chI-EGFP-WPRE-SV40p(A) (4.7*10E13 vg/ml) (Viral Vector Facility VVF, Institute of Pharmacology and Toxicology, University of Zurich or Viral Core at Boston Children's Hospital see below) was injected. And for the therapeutic virus, AAV2/Php.B-CBA-VGLUT3-WPRE (2.27E13) (Viral Core at Boston Children's Hospital (BCH)) was used. The AAVs were injected with a controlled rate of 1 ul/min and to minimize backflow the needle and the glue were only removed 30 minutes after the end of injection. The skin was then sutured with a 6-0 absorbable suture line (Vicryl, Ethicon) and upon awakening, mice were administered with Carprofen (5 mg/kg, S.C.) and finally returned to their cages. The mice received daily doses of Carprofen until 72 h post-surgery. The eGFP injected mice were euthanized after a 2 week survival period and transcardially perfused as described below. The therapeutic virus injected mice the auditory brainstem response was measured 2 weeks post injection.

### Microspheres

The mice were subjected to CM cannulation as described above. Prior to the injection amine modified microspheres (Thermofisher) were diluted in ACSF in a 1:3 ratio and submitted to ultrasonic vibration for 5 minutes. Then they were injected with an infusion rate of 1 ul/min and allowed to circulate for 1 h and 24 h before the animals were perfused and the tissue was processed as described below.

### Histology and Immunohistochemistry

### Perfusion

Prox1-EGFP+, Slc17A8 mutant, and C57BL/6JRj mice were anesthetized by intraperitoneal injection of a mixture of ketamine and xylazine (100 mg kg/10 mg kg) and perfused transcardially with 10 mL 0.01 M phosphate buffer saline (PBS, pH 7.4, Sigma-Aldrich, St. Louis, Missouri, USA) followed by 30 mL of 4% paraformaldehyde solution (PFA, Sigma-Aldrich) diluted in PBS and pH adjusted to 7.4.

### Virus and Microspheres Injected Animals

### Cryosectioning

After perfusion, microsphere injected animals ears were harvested and put in formaldehyde for 24 h, before storage at 5° C. in PBS. The inner ears were decalcified in 0.12 M ethylenediaminetetraacetic acid EDTA for approx. 4 days before incubation in 20% sucrose in PBS overnight for dehydration. For cryosectioning, the cochleas were then embedded in gelatin, at 37° C. for 3 hours before they were incubated in 20% sucrose for 24 h for dehydration. Then the cochleas were frozen in tissue medium (SPECS) using dry ice and isopentane and sectioned in 10 μm thick sections.

GFP treated cochleae were stained with mouse monoclonal GFP (1:400) for the quantification of the GFP transfected inner hair cells (IHC) and with rabbit polyclonal calbindin (1:400) or rabbit polyclonal Myosin VIIA (1:400) for the delineation of the hair cell bodies. Cochlear samples were incubated with primary antibodies overnight at 37° C. followed by 2 hours incubation at 37° C. with secondary antibodies coupled to Alexa fluor dyes, goat anti-mouse 488 (green, at 1:1000) and goat anti-rabbit 594 or donkey anti-rabbit 594 (red, at 1:1000), correspondingly to primary antibodies. Then cochlear pieces were mounted in Vectashield antifade mounting medium with DAPI (H-1200, Vector Laboratories), cover-slipped and sealed with nail polish.

VGLUT3 virus or aCSF treated (used as a control) cochleae were stained for (a) C-terminal binding protein 2 (mouse IgG1 anti-CtBP2, 612044 from BD-Biosciences, used at 1:200) for the demonstration and quantification of the presynaptic ribbons; (b) Glutamate receptor subunit A2 (mouse IgG2A anti-GluA2, MAB397 from Millipore, used at 1:800), in order to quantify the glutamatergic postsynaptic receptors, (c) anti-myosin VIIA (rabbit IgG, 25-6790 from Proteus Biosciences, used at 1:200) for delineation of the hair cell bodies and (d) anti-vesicular glutamate transporter 3 (guinea-pig, anti-VGLUT3, AB5421-I from Sigma-Aldrich, used at 1:400) for observation of the restoration of the hair cells, overnight at 37° C. Incubation with secondary antibodies coupled to Alexa fluor dyes was followed for 2 hours at 37° C. with (a) goat anti-mouse IgG1 647 (far red, at 1:500), (b) goat anti-mouse IgG2a 555 (red, at 1:500), (c) donkey anti-rabbit IgG 405 (blue, at 1:200) and (d) goat anti-guinea pig IgG 488 (green. At 1:500), correspondingly to primary antibodies. Then cochlear pieces were mounted in Vectashield antifade mounting medium without DAPI (H-1000, Vector Laboratories), cover-slipped and sealed with nail polish.

Next, cochlear frequency mapping was performed using a custom Image-J software plug-in, provided by the NIH (Measure_Line.class from Liberman research group at the Eaton-Peabody laboratory). This mapping provides with the total length of each mouse cochlea and some respective frequency points (apex, middle, base), which were used as guide for obtaining high quality images in the confocal microscope for discrete frequency regions across the whole cochlear length. Z-stacks (20-40), with dimensions 1024×1024 and 16-bit image, were taken with a 63× oil immersion objective (NA 1.40) on a Zeiss LSM 880 confocal microscope. A Z-stack of 642 nm and interval 1 μm was used to capture all pre- and postsynaptic structures, as well as their coupling of at least 10 IHCs.

### Quantification of Pre- and Post-Synaptic Inner Hair Cell Structures

For assessing the total number of ribbons and postsynaptic GluA2 puncta per IHC, surface and masking structures were created. Image stacks were analyzed using Imaris software (×64 9.2.0, Bitplane AG, Zurich, Switzerland) for the number of ribbons, postsynaptic receptors and their synaptic pairing. Then, the region of interest (approximately 10 IHCs) was tagged for counting by using the “spots” function. After adjusting for the thresholds, puncta with pixel intensities 0.7-0.8 μm on an 8-bit scale (0-255) were counted. For determining the synaptic pairing, “distance transformation” and “spot co-localization” was used. The threshold/distance between the spots was set to 1 μm.

### Whole Heads for Lymphatic Marker Analysis

The entire mice and torso of perfusion-fixed Prox1-EGFP+ mice were decalcified during three weeks with 10% EDTA in Tris buffer (pH 7) at room temperature, prior to paraffin embedding and serial sectioning. Sections were processed for immunohistochemistry as described above.

### Immunohistochemistry

After PBS washes, histological sections of the ear, brain and other analyzed organs were blocked for one hour at room temperature (RT) in a solution containing 0.3% Triton X-100 (Sigma-Aldrich) and 5% normal donkey or goat serum (Gibco™; Thermo Fisher Scientific, Waltham, Massachusetts, USA) in PBS followed by incubation overnight at 4° C. with primary antibodies (Table S1) diluted in blocking solution. Immunolabeling was revealed by incubation with the appropriate secondary antibodies coupled to fluorophores (Alexa Fluor, 1:500; Invitrogen™ Molecular Probes™; Thermo Fisher Scientific) for two hours at room temperature. DAPI (4′,6-diamidino-2-phenylindole, Thermo Fisher Scientific, 1 g/mL diluted in PBS) was used for nuclear counterstaining and sections were mounted on glass slides using Prolong Gold Antifade Reagent (Invitrogen/Thermo Fisher Scientific, Carlsbad, California, USA). Images of the immunolabeled sections and meninges were acquired on an epifluorescence microscope (Nikon Ni-E) with Plan Apo λ 4×/0.20 objective and on a confocal microscope (Nikon Eclipse Ti, Tokyo, Japan) with Plan Fluor 20×/0.75 and 40×/1.30 oil objectives.

### MR Live Imaging

Immediately after the CM cannulation procedure, animals were moved to MRI scanner and head movement during scanning was minimized by restraining the animals on an MR-compatible stereotactic holder with ear bars. Body temperature was maintained at 37±0.5° C. with a thermostatically controlled waterbed and monitored, along with the respiratory rate by an MR compatible remote monitoring system (SA Instruments, NY, USA). MRI was performed in a 9.4 T preclinical scanner (BioSpec 94/30 USR, Paravision 6.0.1 software, Bruker BioSpin, Ettlingen, Germany) equipped with a 1H cryogenically-cooled quadrature-resonator Tx/Rx coil (CryoProbe, Bruker) and 240 mT/m gradient coil (BGA-12S, Bruker). The modified scanning procedure was performed as previously described (Stanton et al., 2021). T2-weighted structural image was conducted using 3D constructive interference steady-state (3D-CISS). Every 3D-CISS image was calculated as a maximum intensity projection from 4 realigned 3D-TrueFISP volumes with 4 orthogonal phase encoding directions (TR/TE 3.9/1.95 ms, Nex 2, FA 50°, FOV 19.2×12.8×12.8 mm, Matrix 192×128×128, BW 150 kHz). Flow-compensated 3D time-of-flight MR Angiography (3D-TOF-MRA) was collected with FLASH sequence (TR/TE 10/1.95 ms, Nex 2, FA 20°, FOV 19.2×15×16 mm, Matrix 246×192×205, BW=100 kHz). For dynamic contrast enhancement MRI (DCE-MRI), pre- and post-contrast T1-weighted imaging were collected with 3D-FISP sequence (TR/TE 4/2 ms, FA 15°, FOV 19.2×12.8×12.8 mm, Matrix 192×128×128, BW 150 kHz). As T1-enhancing contrast agent gadobutrol (20 mM; Gadovist, Bayer Pharma AG, Leverkusen, Germany) was injected into cisterna magna (1 μL/min for 10 min) a DCE-MRI of the entire mouse brain was performed in 1 min or 0.5 min at an isotropic spatial resolution of 100 μm. The time series DCE scanning protocol comprised three baseline scans (3 min) followed by intracisternal infusion. Scans continued over 90-120 measurements (60-90 min).

### CT Live Imaging

During the CM cannulation procedure a custom-made head bar was fixed on the top of the animal skull. The head bar was designed to minimize undesired movements during the image acquisition (Yoshida et al., 2016), and fixed to an animal holder. Immediately after the CM cannulation procedure, animals were moved to CT scanner. Animal's respiration was monitored by using Biovet software. CT scans were performed with the Vector4CT system (MILabs, Utrecht, Netherlands) and scanning of the entire mouse head was performed in 4.56 min at an isotropic spatial resolution of 20 μm. The time series CT scanning protocol comprised one baseline scan (4.5 min) followed by intracisternal infusion of Omnipaque (350 mg I/ml, 1 μL/min, 10 μL, GE healthcare). Scans continued over 6 measurements (30 min) after intracisternal infusion of Omnipaque.

### Image Processing

Both DCE-MRI and CT time series data were motion corrected using Advanced Normalization Tools (ANTs ver.2.1.0) (Avants et al., 2008; Avants et al., 2011). Motion corrected time series in MRI images were converted into percent change from baseline-time series, calculated as the percent signal change from the averaged signal of baseline images. To correct for subject motion between MRI scans, anatomical images (3D-CISS and 3D-TOF-MRA) were registered to the baseline image of DCE-MRI using two rigid registration process in ITK-SNAP (ver. 3.8.0). In the MRI 3D-CISS images were used as anatomical reference to place the volumes of interest (VOIs) in cisterna magna, outside the cochlear aqueduct, in the cochlear aqueduct, and in the cochlea. Hyperintensity of 3D-CISS represented the CSF space and fluid inside the cochlea, therefore above mentioned VOIs were semi-automatic segmented using thresholding based on the signal intensity of 3D-CISS using ITK-SNAP. The VOI was drawn outside the cochlear aqueduct manually based on the tracer accumulation and placement of the cochlear aqueduct elucidated by the maximum intensity projection of DCE-MRI images. CT VOIs placement is described below, and the distances were measured manually in ITK-SNAP.

### CT ROIs Positions are Described Below

| | | |
|---|---|---|
| Cochlear aqueduct 1 | CA1 | Internal opening of the cochlear aqueduct |
| Cochlear aqueduct 2 | CA2 | Sequentially placed VOI towards the cochlea |
| Cochlear aqueduct 3 | CA3 | Sequentially placed VOI towards the cochlea |
| Cochlear aqueduct 4 | CA4 | Sequentially placed VOI towards the cochlea |
| Cochlear aqueduct 5 | CA5 | Internal opening of the cochlear aqueduct |
| Scala tympani 1 | ST1 | Placed in the scala tympani just outside of the cochlear |
| | | aqueduct |
| Scala vestibuli 1 | SV1 | Placed in the scala vestibuli just outside of the cochlear |
| | | aqueduct |
| Scala tympani 2 | ST2 | Placed between ST1 and ST3 in the horizontal plane |
| Scala vestibuli 2 | SV2 | Placed between SV1 and SV3 in the horizontal plane |
| Scala tympani 3 | ST3 | Placed in the most basal part of the scala tympani in the |
| | | sagittal section where the cochlear nerve has the biggest |
| | | diameter |
| Scala vestibuli 3 | SV3 | Placed in the most basal part of the scala vestibuli in the |
| | | sagittal section where the cochlear nerve has the biggest |
| | | diameter |
| Scala tympani 4 | ST4 | Placed in the most basal part of the scala tympani in the |
| | | horizontal section where the cochlear nerve has the biggest |
| | | diameter |
| Scala vestibuli 4 | SV4 | Placed in the most basal part of the scala vestibuli in the |
| | | horizontal section where the cochlear nerve has the biggest |
| | | diameter |
| Scala tympani 5 | ST5 | Placed in the middle part of the scala tympani in the sagittal |
| | | section where the cochlear nerve has the biggest diameter |
| Scala vestibuli 5 | SV5 | Placed in the middle part of the scala vestibuli in the sagittal |
| | | section where the cochlear nerve has the biggest diameter |
| Scala tympani 6 | ST6 | Placed in the middle part of the scala tympani in the horizontal |
| | | section where the cochlear nerve has the biggest diameter |
| Scala vestibuli 6 | SV6 | Placed in the middle part of the scala vestibuli in the |
| | | horizontal section where the cochlear nerve has the biggest |
| | | diameter |
| Scala tympani 7 | ST7 | Placed in the apical part of the scala tympani in the sagittal |
| | | section where the cochlear nerve has the biggest diameter |
| Scala vestibuli 7 | SV7 | Placed in the apical part of the scala vestibuli in the sagittal |
| | | section where the cochlear nerve has the biggest diameter |
| Scala tympani 8 | ST8 | Placed in the apical part of the scala tympani in the horizontal |
| | | section where the cochlear nerve has the biggest diameter |
| Scala vestibuli 8 | SV8 | Placed in the apical part of the scala vestibuli in the horizontal |
| | | section where the cochlear nerve has the biggest diameter |
| Scala media 1 | SM1 | Placed in the scala media just outside of the cochlear |
| | | aqueduct |
| Scala media 2 | SM2 | Placed in scala media in the same sagittal plane as ST2 and |
| | | SV2 |
| Scala media 3 | SM3 | Placed in the most basal part of the scala media in the sagittal |
| | | section where the cochlear nerve has the biggest diameter |
| Scala media 4 | SM4 | Placed in scala media in the same sagittal plane as ST4 and |
| | | SV4 |
| Scala media 5 | SM5 | Placed in scala media in the same sagittal plane as ST5 and |
| | | SV5 |
| Scala media 6 | SM6 | Placed in scala media in the same sagittal plane as ST6 and SV6 |
| Scala media 7 | SM7 | Placed in scala media in the same sagittal plane as ST7 and |
| | | SV7 |
| Scala media 8 | SM8 | Placed in scala media in the same sagittal plane as ST8 and SV8 |
| Cochlear nerve 1 | CN1 | In the middle of the cochlear nerve in the internal opening of |
| | | the internal cochlear channel |
| Cochlear nerve 2 | CN2 | In the cochlear nerve in the apical part of the cochlear |
| Cisterna magna 1 | CM1 | Just in front of the needle on the right side of the needle |
| Cisterna magna 2 | CM2 | Just in front of the needle on the left side of the needle |

Examples of ROI positions are shown in FIG. 8.

### CT

The time-intensity data was extracted using ITK snap with the ROIs described above after background subtraction and then normalized to maximal cisterna magna intensity. Dimensions of cochlear aqueduct were measured using the #X feature in ITK snap for #four animals. Distances between ROIs were calculated using their spatial coordinates.

### MR

#3D image generation with Amira. The background-subtracted images were normalized to maximal cisterna magna intensity.
AQP4 statistical comparison (p-value)
3D image generation

### Dispersion Model

We suggest that the tracer transport through a narrow space such as cochlear aqueduct or scala tympani of length L can be approximated by the dispersion equation

$$dC ( x , t ) / dt = D \backslash nabla C ( x , t ) ,$$

where x is axial distance from the proximal end, t is time and D is the (constant) dispersion coefficient. The boundaries are given Dirichlet conditions by taking measurements at the proximal and distal locations. For the model of cochlear aqueduct,

C(0,t)=c_SAS(t), C(L,t)=c_coch.(t),

where c_SAS and c_coch. are tracer concentrations measured at the proximal and distal end of the cochlear aqueduct. Similarly for scala tympani,

C(0,t)=c_1(t), C(L,t)=c_#N(t),

where subscripts refer to the scala tympani VOI index.

This formulation was derived from Fick's first law using a shell-based approach (Roselli, Diller 2011), assuming flat concentration gradients at the channel boundaries. (We require concentration-measurements to be taken from VOIs of considerably smaller diameter than the axis length.)

To solve the equation (#ref), inventors implemented a simple finite difference scheme in Python (#ref) using NumPy and Scipy (#ref). Estimation of the dispersion coefficient was achieved by minimizing the L2-norm prediction error, using Scipy (#ref) E:

$$E ( D , data ) = sum \backslash \_ observations ( prediction ( x , t ) - observation ( x , t ) ) \hat{} 2.$$

### Viral Vector Production

AAV9-PHP.B vectors carrying the coding sequences of eGFP or mouse VGLUT3 were generated by the Viral Core at Boston Children's Hospital under the authority of BCH Institutional Biosafety Committee (protocol #IBC-P0000047). AM/CBA-VGLUT3-WPRE-BGH plasmids containing mouse VGLUT3 cDNA under control of chicken 3-actin (CBA) promoter and cytomegalovirus (CMV) enhancer (Akil et al., 2012) was kindly provided by Omar Akil. The plasmid was amplified, purified, and sequenced to confirm identity. ITR integrity was assessed by restriction digest, and then submitted to the Viral Core for packaging into AAV9-PHP.B capsids. Similarly, a plasmid containing eGFP driven by CMV promoter was cloned into an AAV2 vector and packaged into AAV9-PHP.B capsid by the Viral Core using a helper virus free system and double transfection method, as previously described (Lee et al., 2020). Vectors were purified via an iodixanol step gradient ultracentrifuge, followed by ion exchange chromatography. The titer of genome-containing particles of AAV9-PHP.B-CBA-VGLUT3-WPRE-BGH was 2.27×1013 gc/mL, assessed by Syber-Green based qPCR assay using primers for bovine growth hormone polyadenylation signal (bGH pA). Two independent batches of AAV9-PHP.B-CMV-eGFP-WPRE were used with titers of 3.54×1012 gc/mL and 4.49×1012 gc/mL, quantified using primers for GFP.

Virus Aliquots were Maintained at −80° C. Until Time of Use.

Auditory brainstem response and distortion product otoacoustic emissions Signal generation and acquisition was done using Tucker-Davis Technologies System III hardware and software. Stimuli were generated using BioSigRZ software running on a PC connected to a signal processor (RZ6). For the distortion product of otoacoustic emissions (DPOAE), two independently driven MF1 speakers merged in a custom made one was used for closed field stimulation. For the auditory brainstem responses (ABR), the stimulus was the output through an open field speaker (MF1). A Brüel and Kjær ¼-inch microphone and a conditioning pre-amplifier (4939 A 011 and 2690 A 0S1) was used to calibrate the stimulus level. Speakers were calibrated once at a time using a frequency sweep (4-32 kHz). The output was corrected to produce a flat spectrum at 90 dB SPL (open field speaker, ABR) and 80 dB SPL (closed field speakers, DPOAE). Mice were anesthetized with a mixture of ketamine (ketaminol 50 mg/ml, Intervet, 511485) and xylazine (Rompun 20 mg/ml, Bayer, KP0A43D) (100 and 10 mg/kg body weight, respectively) and placed in a custom-made acoustic enclosure with sound absorbing material on the walls and ceiling. Body temperature was maintained at 36.5° C. using a heating pad (Homeothermic Monitoring System 55-7020, Harvard Apparatus). First DPOAEs were recorded. In short, the acoustic coupler was inserted into the ear canal. A microphone (EK 23103, Knowles) was inserted in an acoustic coupler connected to a pre-amplifier (ER-10B+, Etymotic Research) and a processor (200 kHz sample rate) to measure sound level in the ear canal. Each speaker played one of two primary tones (f1 and f2) and swept in 5 dB steps from 80-10 dB SPL (for f2). The 2f1-f2 distortion product was measured with f2=8, 12, 16, 24, 32 kHz, f2/f1=1.25, and stimulus levels L1=L2+10 dB SPL. Subsequently, ABRs were measured. Stainless-steel subdermal needle electrodes were placed at the head vertex (positive), under the right ear pinna (negative) and above the right leg (ground). ABRs were evoked by tone bursts (0.5 ms rise/fall time, 5 ms duration) of 8, 12, 16, 24 and 32 kHz presented 21 times per second. Signals were collected via a low-impedance head stage (RA4LI) connected to a pre-amplifier (RA4PA) and digitally sampled (200 kHz sample rate). Responses to 200-3000 bursts were bandpass filtered at 0.3-3 kHz using BioSigRZ and averaged at each level. For each frequency, sound level decreased from 90 dB SPL in 5 Db steps. DPOAEs and ABRs were recorded 2-3 days before the cisterna magna viral injections (baseline) and 14 days after the viral injections (2 w), at which time point animals were euthanized for cochlear histology.

### Example 2 CSF Solutes Enters the Inner Ear Via the Cochlear Aqueduct

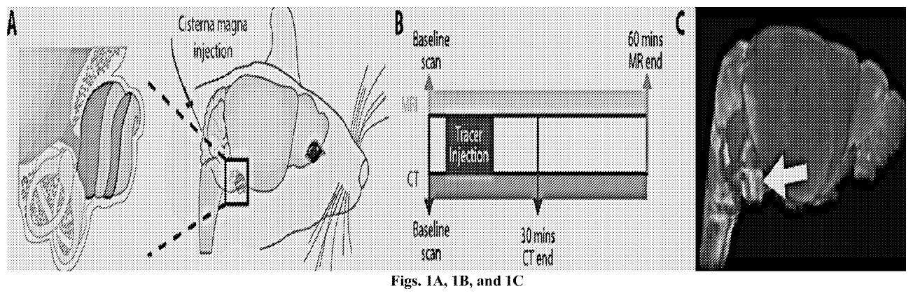

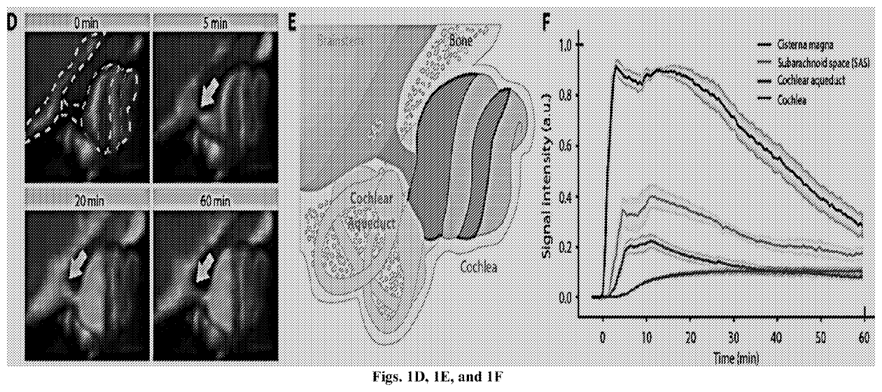

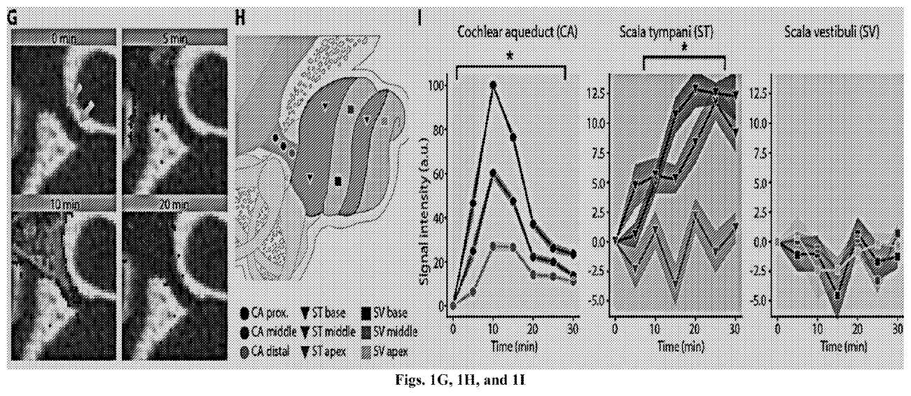

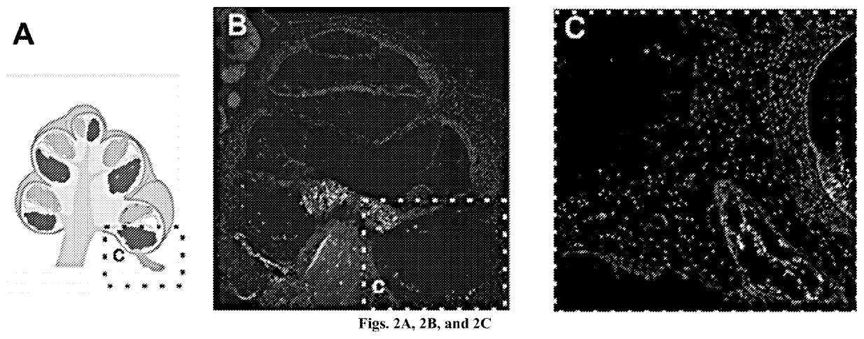

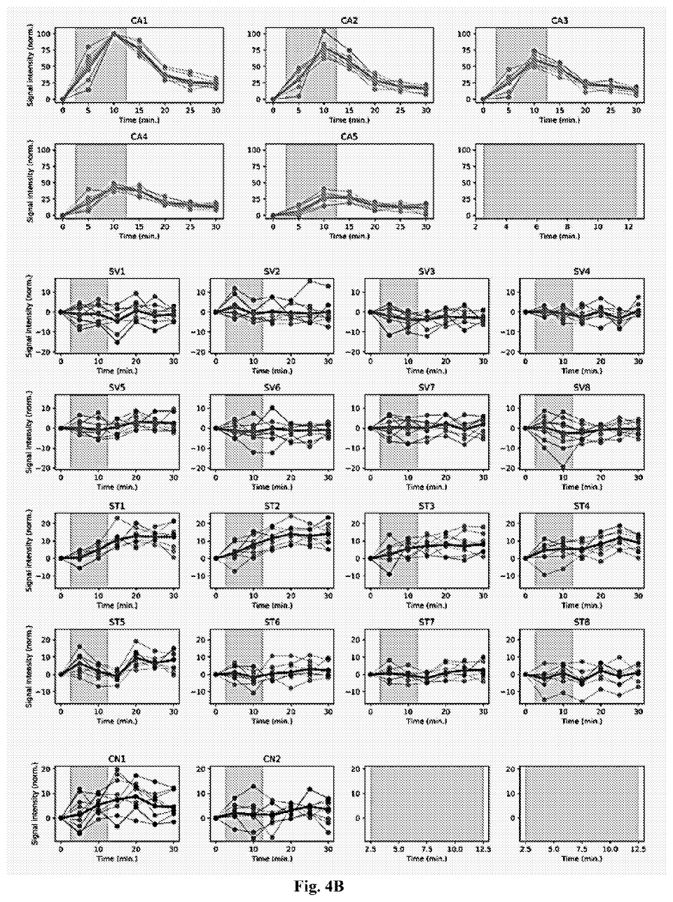

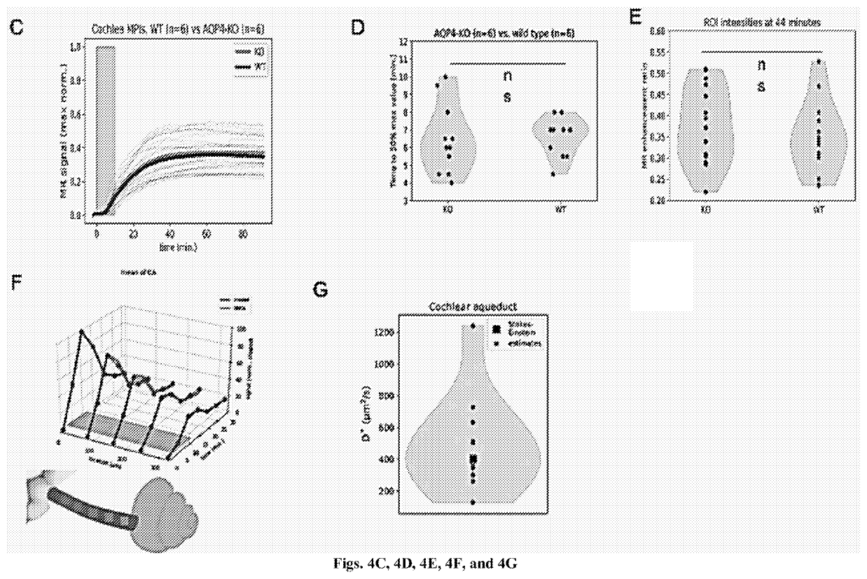

To evaluate whether the inner ear is accessible to CSF tracers in wildtype adult mice, dynamic contrast enhanced magnetic resonance imaging (DCE-MRI) was utilized to visualize CSF transport of Gadovist (0.6 kDa). Real time imaging showed that Gadovist (0.6 kDa) injected into the CSF-filled space of cisterna magna (CM) reaches the cochlea and identified a CSF-filled channel linking the subarachnoid space with cochlea, which is consistent with the anatomical localization of cochlear aqueduct (FIG. 1 A-D). Consistently with that the cochlear aqueduct is the point of entry, the Gadovist signal first rises just outside the cochlear aqueduct, then in cochlear aqueduct and lastly in cochlea (FIGS. 1D-F, S1A). The time to reach 50 max was significantly higher in the cochlea compared to all other groups (FIG. 4A).

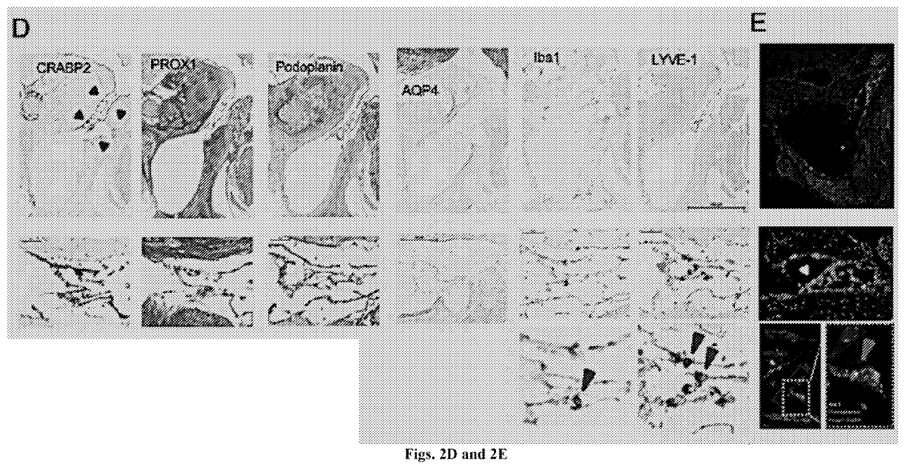

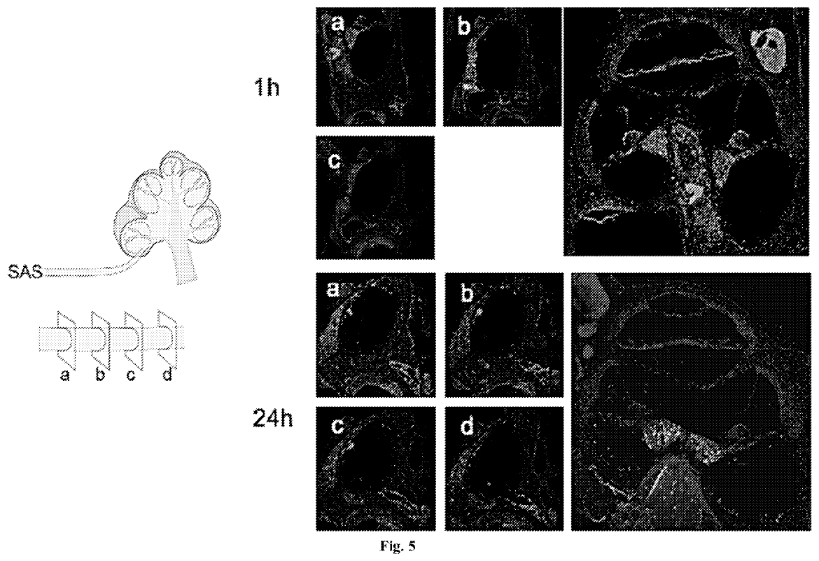

To define if the cochlear nerve contributes to the entry of tracers into cochlea, high spatial resolution computed tomography (CT) scans were performed. Omnipaque (0.8 kDa) was delivered intracisternally, and continuous CT scanning confirmed that tracer concentration rises and reaches the highest concentration in the initial segment of the cochlear aqueduct first. The tracer then disperses further into the aqueduct before it reaches the base and middle region of the scala tympani (FIG. 1G-I). No clear rise in tracer concentration was detectable in scala vestibuli or in the cochlear nerve during the 30 min of scanning (FIGS. 1I, 4B). Thus, CT scanning showed that the aqueduct is the main point of CSF tracer influx. To determine the accessibility of larger CSF tracers, animals were euthanized 1 and 24 hours after CSF delivery of microspheres (0.2 μm). Histological analysis showed that the microspheres were abundant in cochlea aqueduct at both timepoints and were detected in the scala tympani after 24 hours (FIGS. 2A and 5). Combined, this analysis shows that the cochlear aqueduct is directly linking the base of scala tympani to the large CSF-filled subarachnoid space.

### Example 4 Agp4-Independent Dispersive Mechanisms Drive CSF Transport into Inner Ear

Because glymphatic fluid transport is facilitated by the water channel, Aquaporin-4 (AQP4), next it was asked if of AQP4 plays a role in CSF influx into the inner ear. CSF influx into the aqp4-knock out and wildtype littermates were compared after cisterna magna injection of Gadovist. Time activity curves for Gadovist influx in mice lacking aqp4 did not differ from wild type animals demonstrating that the fluid flow was AQP4-independent (FIGS. 4A, C-D.). In fact, immunolabeling supported these observations by showing that the cells lining the cochlear aqueduct were AQP4 negative (FIG. 2F). To understand the mechanisms driving CSF transport into the inner ear, CT time activity curves were used for cochlea aqueduct and scala tympani and anatomical measurements to estimate effective diffusion constants. Deriving a dispersion-based model using Fick's second law correspond to estimated effective diffusion coefficients similar to pure diffusion for the tracer, possible because the lymphatic-like membrane minimized the contribution of advection and dispersion. This suggests that the driving mechanism for CSF transport to the ear is an aqp4-independent dispersive transport.

### Example 3 the Cochlear Aqueduct Exhibits Lymphatic-Like Characteristics

As tracers easily enters the ear, it was hypothesized that the cochlear aqueduct shares similarities to Schlemm's canal that drains aqueous humor from the anterior chamber of the eye (Aspelund et al., 2014). The narrow bony cochlear canal with a diameter of approximately 126 micrometers (Table 1), exhibited an adjacent thin dural layer covered by a very narrow claudin 11-positive arachnoid barrier cell layer. This canal was lined with a tubular evagination of a membrane that extended down to its termination at the entrance to cochlea, a distance of 439±39 micrometers (Table 1). The membrane terminated as a diaphragm-like structure with an accumulation of macrophage-like cells on the cerebral side of the diaphragm. The membrane was positively stained for CRABP2 (cellular retinoic acid binding protein 2) and the lymphatic markers ‘prospero homeobox protein 1 (Prox1) and podoplanin, but not for VEGFR3 or lymphatic hyaluronan receptor (LYVE1) (FIG. 2D). Prox1 is the master regulator of lymphatic endothelial cell fate and lymph vessels expressing Prox1 has been shown to contribute to drainage of macromolecules from the CSF (Louveau et al., 2018-refer to 2015 Nature paper). Schlemm's canal in the eye is similar Prox-1 and podoplanin positive, but LYVE-1 and VEGFR3 negative (Aspelund et al., 2014). LYVE1 and Iba1 stained macrophages were associated with the membrane, but the cells forming the membrane was LYVE1-negative. Iba1 staining of the microsphere images, indicated that the Iba1-positve macrophages phagocyte the microspheres in the channel (FIG. 2E).

### Example 5 Viruses Injected in Cisterna Magna are Expressed in the Inner Ear

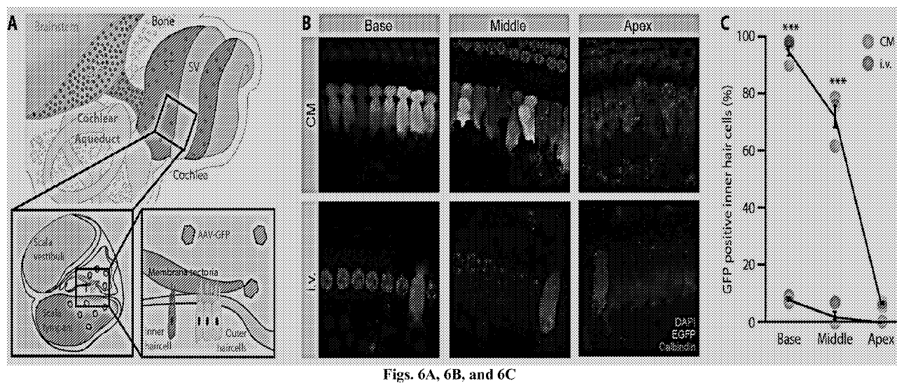

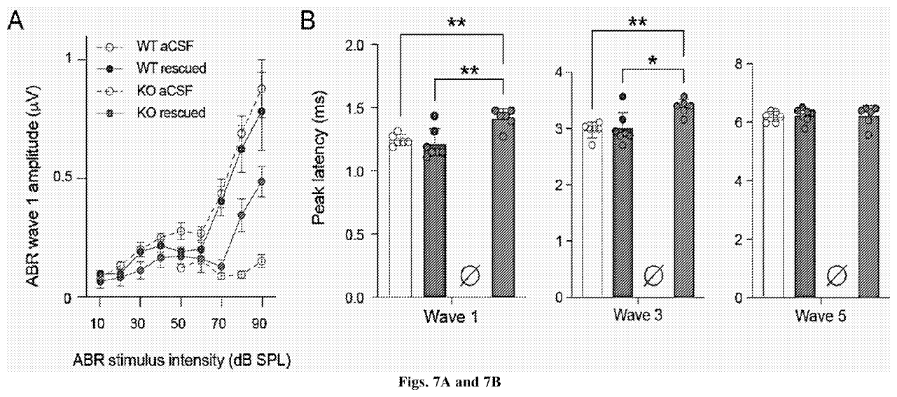

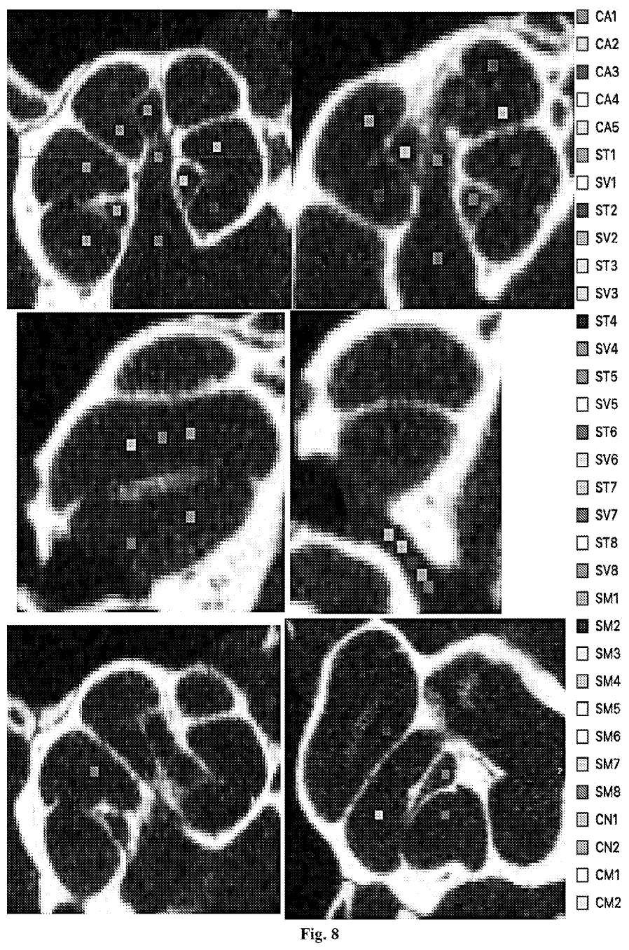

Can the cochlea aqueduct CSF route be used as an alternate strategy for delivery of a viral construct to the inner ear? The reporter virus, AAV-PHP.B-CMV-EGFP has previously been shown to induce robust expression in hair cells when injected directly into the ear (Lee et al., 2020). Adolescence mice received a single injection of AAV-PHP.B-CMV-EGFP and the inner ears were harvested 14 days later. The inner hair cells were successfully transduced by the virus with a gradient of expression more robust in the base of the cochlea and decreasing towards the apex in both number of transfected cells and the signal intensity (FIG. 6A-B). This distribution is consistent with the hypothesis that the virus enters the inner ear through the aqueduct (FIG. 6A). To control for BBB permeability, a subgroup of age-matched mice received AAV-PHP.B-CMV-EGFP intravenously and the inner ears exhibited scant but evenly distributed virus expression when compared to CSF delivery of the same construct (FIG. 6A-B). In sum, CSF can be used a delivery route for viruses into the inner ear.

### Example 6 CM-Delivery of VGLUT3 Expressing Viruses Rescues Hearing of a Mouse Model of DFNA25

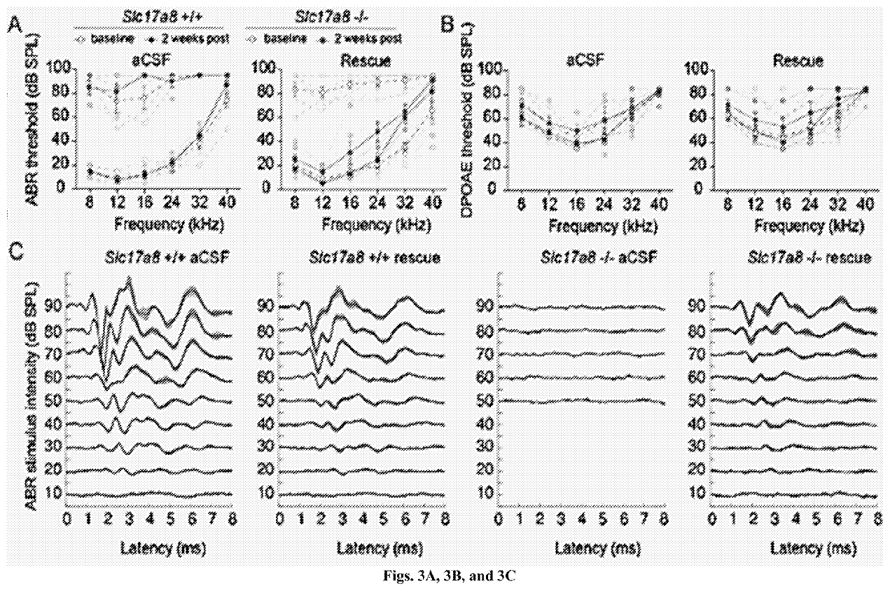

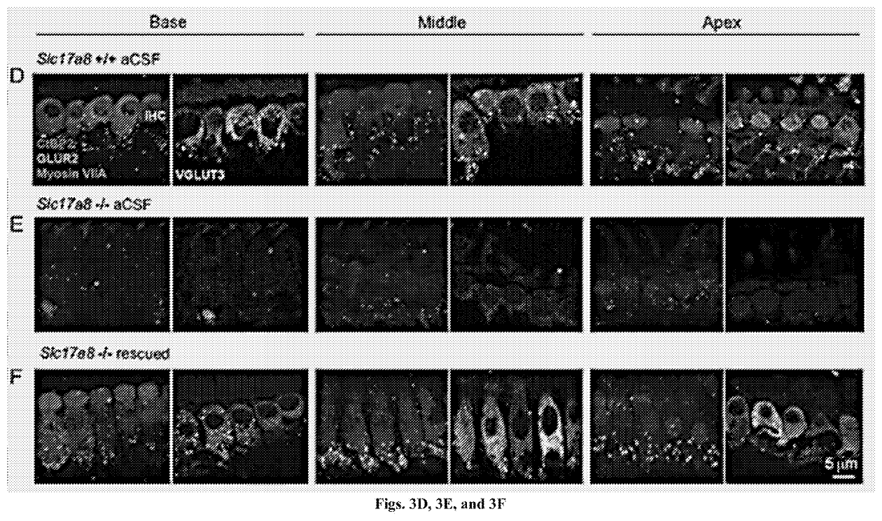

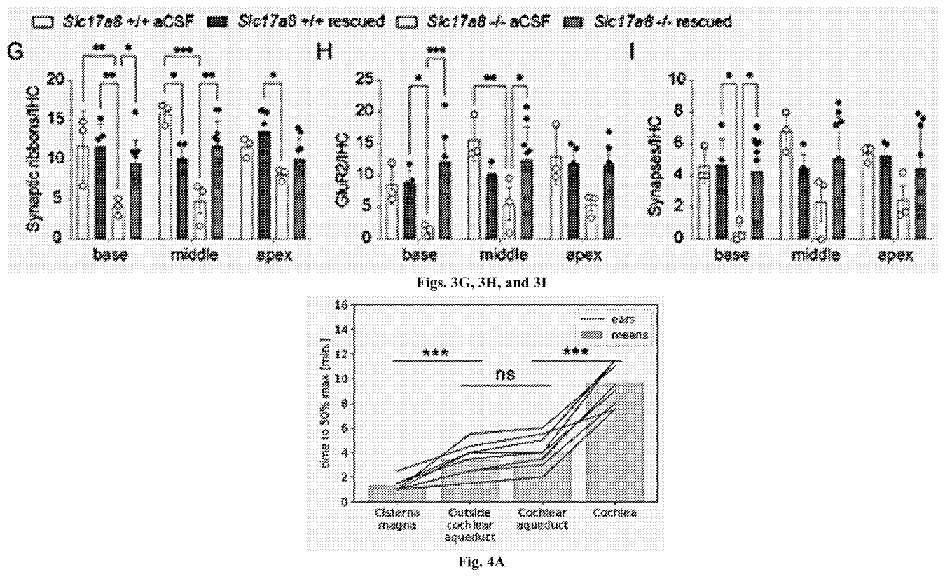

After determining the CSF-route as an effective paradigm to virally transfect inner hair cells, it was evaluated whether viral-mediated overexpression in inner hairs of Slc17a8, encoding the vesicular glutamate transporter-3 (VGLUT3), could rescue hearing in 2 months-old adult Slc17a8 −/− mice, a mouse model for DFNA25, an autosomal-dominant and progressive sensorineural hearing loss (Ruel et al., 2008). VGLUT3 transports glutamate into synaptic vesicles before it is released into the synaptic cleft and is essential for transduction of signals from the inner hair cells. Slc17a8 −/− mice are deaf due to a defect of inner hair cells to uptake and release glutamate to their afferent neurons, while the function of the outer hair cells remains unaffected (Ruel et al., 2008). Auditory brainstem response and distortion product otoacoustic emissions baseline thresholds were measured in wild-type and Slc17a8 −/− mice 7 either before aCSF or viral delivery of either EGFP or VGLUT3 expressing AAV, as well as 2 weeks post-injections (FIG. 3). Mean auditory brainstem response baseline values for the WT ranged from 8-20 dB SPL between 8 and 24 kHz, whereas at 32 and 40 kHz, the mean values were higher (between 38 and 74 dB SPL), a known effect seen in C57BL/6J mice due to a variant in the age-related hearing loss gene (ahl gene) known to cause high-frequency hearing loss by two months of age (Johnson et al., 1997) (FIG. 3A). In comparison, VGLUT3 KO mice had mean elevated baseline thresholds ranging from 74-95 dB SPL. DPOAEs were normal in both WT and KO mice (FIG. 3B). Two weeks after administration of either vehicle (aCSF) or a reporter gene (EGFP-AAV), no changes in hearing thresholds were found, confirming that the intracisternal injections do not impact hearing negatively. In wildtype mice treated with VGLUT3 overexpressing AAVs, thresholds were elevated at 32 and 40 kHz by 25 and 16 dB, respectively when compared to baseline. In contrast, VGlut3 KO mice displayed improvements in hearing thresholds at all frequencies except 48 kHz. In contrast, the function of outer hair cells, as measured by DPOAEs, was not affected by the aCSF or viral rescue in neither the WT nor the KO mice (FIG. 3B).

Averaged wave forms provide a visual imprint of the degree of hearing rescue obtained in the Slc17a8 −/− mice after viral transfection of VGlut3 (FIG. 3C). Overall, the CSF-delivery of VGLUT3 expressing AAV resulted in restoration of evoked-responses in the adult Slc17a8 −/− mice, pointing to that glutamatergic signaling in the inner hair is repaired. However, in spite of hearing thresholds reaching normal levels, ABR wave 1 amplitude was restored to 62% of the WT mice treated with VGLUT3-AAV (FIG. 3e). Latency of Wave 1 and 3 was also found delayed by 0.2 ms (p=0.002) and 0.38 ms (p=0.0112) respectively, in rescued Slc17a8 −/− compared with Slc17a8+/+ mice (FIG. 3), while Wave 5 latency was not affected. These findings suggest that although hearing thresholds were fully restored in Slc17a8 −/− mice, some degree of neural asynchrony remained two weeks after CSF delivery of VGLUT3 expressing AAVs.

To determine the degree of synapse repair by the viral rescue, inventors quantified the number of paired synapses between the inner hair cells and the afferent dendrites using high-resolution immunocytochemical procedures against ribbons (CtBP2) and the AMPA subunit GluR2 in the apical, middle and basal cochlear regions. Representative micrographs from the different groups at each cochlear location is illustrated (FIG. 3G). Viral rescue did not affect the number of individual pre- and post-synaptic entities, nor their pairing in Slc17a8 −/− mice. However, VGLUT3-AAV increased the number of ribbons and GluR2 units, in middle and base turns, while pairing of the synapses was fully recovered in the basal region (see Supplementary Table 1 for statistical analysis, FIG. 3).

### DISCUSSION

The auditory organs are traditionally considered to be a part of the peripheral nervous system. The inner ear together with the cochleovestibular ganglion is derived from the surface ectoderm rather than the neuroectoderm that gives rise to the central nervous system (Freyer et al., 2011). Yet fluids of the ear and brain are connected via the cochlear aqueduct, and solutes are within minutes transported from the CSF-filled subarachnoid space to the inner ear. It was shown here that large viral constructs successfully are delivered to the ear through the cochlear aqueduct and restored hearing in deaf adult mice. The transport is driven by dispersion, in contrast to the advective Aqp4-dependent glymphatic transport of the brain. A pseudo-lymphatic membrane was identified in the cochlear aqueduct, that appeared to restrict transport between the ear and brain, as evidenced by extensive phagocytosis of CSF microspheres by macrophages residing in the membrane. Nevertheless, reporter genes and VGlut3 were readily expressed by the inner hair cells after intracisternal CSF administration. The successful delivery of viral gene therapy to treat hearing loss in the adult cochlea via CSF has broad translational implications. Cisterna magna injections are non-damaging to the cochlea, in contrast to traditional routes of direct injection through the round window membrane or cochlea. Since modern interventional radiology has rendered cisternal administration a relatively safe procedure, CSF delivery of gene therapeutics will facilitate the treatment of genetically-driven and congenital hearing loss. While the study assessed hearing recovery 2 weeks post-treatment, others have shown durable AAV rescue of hearing up to a year after injection through the round window membrane (Gyorgy et al., 2019). In addition, intracisternal injection can be performed multiple times without a risk for cochlear damage, as opposed to round window injections. Overall, the study provides a proof-of-concept of a novel, relatively non-invasive and highly effective route for gene therapy of hearing disorders.

SUPPLEMENTARY TABLE 1
Cochlear aqueduct dimensions. The cochlear
aqueduct dimensions measured in the CT.

| | | |
|---|---|---|
| Length | 439 ± 39 μm | 373-490 μm |
| cochlear end width | 174 ± 23 μm | 141-204 μm |
| Middle width | 126 ± 15 μm | 106-154 μm |
| SAS end width | 365 ± 63 μm | 260-477 μm |

SUPPLEMENTARY TABLE 2
Statistical reporting of FIG. 3. Three-way ANOVA from Ribbon. GluR2 and synaptic
pairing in Slc17a8 +/+ and −/− mice treated with aCSF or rescued.

| ANOVA table | SS (Type III) | DF | MS | F (DFn, DFd) | P value |
|---|---|---|---|---|---|
| Ribbons | | | | | |
| Region | 24.55 | 2 | 12.28 | F (2, 49) = 1.659 | P = 0.2008 |
| Genotype | 237.8 | 1 | 237.8 | F (1, 49) = 32.14 | P < 0.0001 |
| Treatment | 38.64 | 1 | 38.64 | F (1, 49) = 5.223 | P = 0.0267 |
| Region × Genotype | 4.553 | 2 | 2.277 | F (2, 49) = 0.3077 | P = 0.7366 |
| Region × Treatment | 9.69 | 2 | 4.845 | F (2, 49) = 0.6549 | P = 0.5240 |
| Genotype × | 113 | 1 | 113 | F (1, 49) = 15.28 | P = 0.0003 |
| Treatment | | | | | |
| Region × Genotype × | 84.03 | 2 | 42.01 | F (2, 49) = 5.679 | P = 0.0061 |
| Treatment | | | | | |
| GluR2 | | | | | |
| Region | 97.09 | 2 | 48.54 | F (2, 49) = 3.739 | P = 0.0308 |
| Genotype | 124 | 1 | 124 | F (1, 49) = 9.546 | P = 0.0033 |
| Treatment | 99.92 | 1 | 99.92 | F (1, 49) = 7.695 | P = 0.0078 |
| Region × Genotype | 11.64 | 2 | 5.82 | F (2, 49) = 0.4482 | P = 0.6413 |
| Region × Treatment | 48.95 | 2 | 24.48 | F (2, 49) = 1.885 | P = 0.1627 |
| Genotype × | 318.4 | 1 | 318.4 | F (1, 49) = 24.52 | P<0.0001 |
| Treatment | | | | | |
| Region × Genotype × | 17.41 | 2 | 8.704 | F (2, 49) = 0.6703 | P = 0.5162 |
| Treatment | | | | | |
| Residual | 636.2 | 49 | 12.98 | | |
| Paired synapses | | | | | |
| Region | 11.78 | 2 | 5.892 | F (2, 49) = 1.419 | P = 0.2517 |
| Genotype | 51.18 | 1 | 51.18 | F (1, 49) = 12.33 | P = 0.0010 |
| Treatment | 13.31 | 1 | 13.31 | F (1, 49) = 3.206 | P = 0.0796 |
| Region × Genotype | 0.4871 | 2 | 0.2435 | F (2, 49) = 0.05866 | P = 0.9431 |
| Region × Treatment | 6.288 | 2 | 3.144 | F (2, 49) = 0.7572 | P = 0.4744 |
| Genotype × | 40.68 | 1 | 40.68 | F (1, 49) = 9.798 | P = 0.0029 |
| Treatment | | | | | |
| Region × Genotype × | 4.256 | 2 | 2.128 | F (2, 49) = 0.5125 | P = 0.6022 |
| Treatment | | | | | |
| Residual | 203.5 | 49 | 4.152 | | |

### REFERENCES

Ahn, J. H., Cho, H., Kim, J. H., Kim, S. H., Ham, J. S., Park, I., Suh, S. H., Hong, S. P., Song, J. H., Hong, Y. K., Jeong, Y., Park, S. H., & Koh, G. Y. (2019). Meningeal lymphatic vessels at the skull base drain cerebrospinal fluid. Nature, 572(7767), 62-66.
Akil, O., Dyka, F., Calvet, C., Emptoz, A., Lahlou, G., Nouaille, S., Boutet de Monvel, J., Hardelin, J. P., Hauswirth, W. W., Avan, P., Petit, C., Safieddine, S., & Lustig, L. R. (2019). Dual AAV-mediated gene therapy restores hearing in a DFNB9 mouse model. Proc Natl Acad Sci USA, 116(10), 4496-4501.
Albernaz, P. L. (2016). Hearing Loss, Dizziness, and Carbohydrate Metabolism. Int Arch Otorhinolaryngol, 20(3), 261-270.
Aspelund, A., Tammela, T., Antila, S., Nurmi, H., Leppanen, V. M., Zarkada, G., Stanczuk, L., Francois, M., Makinen, T., Saharinen, P., Immonen, I., & Alitalo, K. (2014). The Schlemm's canal is a VEGF-C/VEGFR-3-responsive lymphatic-like vessel. J Clin Invest, 124(9), 3975-3986.
Bankoti, K., Generotti, C., Hwa, T., Wang, L., O'Malley, B. W., Jr., & Li, D. (2021). Advances and challenges in adeno-associated viral inner-ear gene therapy for sensorineural hearing loss. Mol Ther Methods Clin Dev, 21, 209-236.
Choi, I., Chung, H. K., Ramu, S., Lee, H. N., Kim, K. E., Lee, S., Yoo, J., Choi, D., Lee, Y. S., Aguilar, B., & Hong, Y. K. (2011). Visualization of lymphatic vessels by Prox1-promoter directed GFP reporter in a bacterial artificial chromosome-based transgenic mouse. Blood, 117(1), 362-365.
Collaborators, G. B. D. H. L. (2021). Hearing loss prevalence and years lived with disability, 1990-2019: findings from the Global Burden of Disease Study 2019. Lancet, 397(10278), 996-1009.
Dulon, D., Papal, S., Patni, P., Cortese, M., Vincent, P. F., Tertrais, M., Emptoz, A., Tlili, A., Bouleau, Y., Michel, V., Delmaghani, S., Aghaie, A., Pepermans, E., Alegria-Prevot, O., Akil, O., Lustig, L., Avan, P., Safieddine, S., Petit, C., & El-Amraoui, A. (2018). Clarin-1 gene transfer rescues auditory synaptopathy in model of Usher syndrome. J Clin Invest, 128(8), 3382-3401.
Ekdale, E. G. (2016). Form and function of the mammalian inner ear. J Anat, 228(2), 324-337.
Gyorgy, B., Nist-Lund, C., Pan, B., Asai, Y., Karavitaki, K. D., Kleinstiver, B. P., Garcia, S. P., Zaborowski, M. P., Solanes, P., Spataro, S., Schneider, B. L., Joung, J. K., Geleoc, G. S. G., Holt, J. R., & Corey, D. P. (2019). Allele-specific gene editing prevents deafness in a model of dominant progressive hearing loss. Nat Med, 25(7), 1123-1130.
Iliff, J. J., Wang, M., Liao, Y., Plogg, B. A., Peng, W., Gundersen, G. A., Benveniste, H., Vates, G. E., Deane, R., Goldman, S. A., Nagelhus, E. A., & Nedergaard, M. (2012). A paravascular pathway facilitates CSF flow through the brain parenchyma and the clearance of interstitial solutes, including amyloid beta. Sci Transl Med, 4(147), 147ra111.
Johnson, K. R., Erway, L. C., Cook, S. A., Willott, J. F., & Zheng, Q. Y. (1997). A major gene affecting age-related hearing loss in C57BL/6J mice. Hearing Research, 114(1-2), 83-92.
Lee, J., Nist-Lund, C., Solanes, P., Goldberg, H., Wu, J., Pan, B., Schneider, B. L., & Holt, J. R. (2020). Efficient viral transduction in mouse inner ear hair cells with utricle injection and AAV9-PHP.B. Hear Res, 394, 107882.
Lilius, T. O., Blomqvist, K., Hauglund, N. L., Liu, G., Staeger, F. F., Baerentzen, S., Du, T., Ahlstrom, F., Backman, J. T., Kalso, E. A., Rauhala, P. V., & Nedergaard, M. (2019). Dexmedetomidine enhances glymphatic brain delivery of intrathecally administered drugs. J Control Release, 304, 29-38.
Louveau, A., Herz, J., Alme, M. N., Salvador, A. F., Dong, M. Q., Viar, K. E., Herod, S. G., Knopp, J., Setliff, J. C., Lupi, A. L., Da Mesquita, S., Frost, E. L., Gaultier, A., Harris, T. H., Cao, R., Hu, S., Lukens, J. R., Smirnov, I., Overall, C. C., . . . Kipnis, J. (2018). CNS lymphatic drainage and neuroinflammation are regulated by meningeal lymphatic vasculature. Nat Neurosci, 21(10), 1380-1391.
Nyberg, S., Abbott, N. J., Shi, X., Steyger, P. S., & Dabdoub, A. (2019). Delivery of therapeutics to the inner ear: The challenge of the blood-labyrinth barrier. Sci Transl Med, 11(482).
Plog, B. A., Mestre, H., Olveda, G. E., Sweeney, A. M., Kenney, H. M., Cove, A., Dholakia, K. Y., Tithof, J., Nevins, T. D., Lundgaard, I., Du, T., Kelley, D. H., & Nedergaard, M. (2018). Transcranial optical imaging reveals a pathway for optimizing the delivery of immunotherapeutics to the brain. JCI Insight, 3(20).
Salt, A. N., Gill, R. M., & Hartsock, J. J. (2015). Perilymph Kinetics of FITC-Dextran Reveals Homeostasis Dominated by the Cochlear Aqueduct and Cerebrospinal Fluid. J Assoc Res Otolaryngol, 16(3), 357-371.
Spinelli, K. J., Klimek, J. E., Wilmarth, P. A., Shin, J. B., Choi, D., David, L. L., & Gillespie, P. G. (2012). Distinct energy metabolism of auditory and vestibular sensory epithelia revealed by quantitative mass spectrometry using MS2 intensity. Proc Natl Acad Sci USA, 109(5), E268-277.
Vogl, C., Panou, I., Yamanbaeva, G., Wichmann, C., Mangosing, S. J., Vilardi, F., Indzhykulian, A. A., Pangrsic, T., Santarelli, R., Rodriguez-Ballesteros, M., Weber, T., Jung, S., Cardenas, E., Wu, X., Wojcik, S. M., Kwan, K. Y., Del Castillo, I., Schwappach, B., Strenzke, N., . . . Moser, T. (2016). Tryptophan-rich basic protein (WRB) mediates insertion of the tail-anchored protein otoferlin and is required for hair cell exocytosis and hearing. EMBO J, 35(23), 2536-2552.
Wang, X., Lou, N., Eberhardt, A., Yang, Y., Kusk, P., Xu, Q., Forstera, B., Peng, S., Shi, M., Ladron-de-Guevara, A., Delle, C., Sigurdsson, B., Xavier, A. L. R., Erturk, A., Libby, R. T., Chen, L., Thrane, A. S., & Nedergaard, M. (2020). An ocular glymphatic clearance system removes beta-amyloid from the rodent eye. Sci Transl Med, 12(536).
Xie, L., Kang, H., Xu, Q., Chen, M. J., Liao, Y., Thiyagarajan, M., O'Donnell, J., Christensen, D. J., Nicholson, C., Iliff, J. J., Takano, T., Deane, R., & Nedergaard, M. (2013). Sleep drives metabolite clearance from the adult brain. Science, 342(6156), 373-377.
Aspelund, A., Tammela, T., Antila, S., Nurmi, H., Leppanen, V. M., Zarkada, G., Stanczuk, L., Francois, M., Makinen, T., Saharinen, P., Immonen, I., & Alitalo, K. (2014). The Schlemm's canal is a VEGF-C/VEGFR-3-responsive lymphatic-like vessel. J Clin Invest, 124(9), 3975-3986.
Freyer, L., Aggarwal, V., & Morrow, B. E. (2011). Dual embryonic origin of the mammalian otic vesicle forming the inner ear. Development, 138(24), 5403-5414.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method for delivering an agent to an inner ear of a subject comprising administering the agent to the cerebrospinal fluid (CSF) in the subject.

2. The method of claim 1, wherein the agent is a therapeutic agent.

3. The method of claim 2, wherein the therapeutic agent is selected from the group consisting of a small molecule, a large molecule, a peptide, a protein, an antibody, a nucleic acid, a vector, and a cell.

4. The method of claim 3, wherein the therapeutic agent is the nucleic acid or the vector.

5. The method of claim 4, wherein the nucleic acid or the vector comprises a gene that encodes a protein or polypeptide.

6. The method of claim 5, wherein the gene is selected from the group consisting of VGLUT3, MYO7A (USH1B), USH1C, CDH23, PCDH15 (USH1F), SANS (USH1G), USH2A, ADGRV1/VLGR1, WHRN (DFNB31), USH3A (CLRN1), HARS, Cx26 (GJB2), Cx30 (GJB6), Cx29 (GJC3), Cx31 (GJB3), ACTG1, FSCN2, RDX, POU4F3, TRIOBP, TPRN, XIRP2, ATOH1, GFI1, CHRNA9, CIB3, CDH23, PCDH15, KNCN, DFNB59, OTOF, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, CABP2, EPS8, EPS8L2, ESPN, ESPNL, PRPH2, STRC, SLC8A2, ZCCHC12, LRTOMT2, LRTOMT1, USH1C, ELFN1, TTC24, DYTN, KCP, CCER2, LRTM2, KCNA10, NTF3, CLRN1, CLRN2, SKOR1, TCTEX1 D1, FCRLB, SLC17A8, GRXCR2, BDNF, SERPINE3, NHLH1, HSP70, HSP90, ATF6, PERK, IRE1, BIP, GJB2, and USH1G.

7. The method of claim 2, wherein the therapeutic agent is selected from the group consisting of a Jun N terminal kinase inhibitor, a brain derived neurotrophic factor ligand, a PPAR agonist, a gamma-secretase inhibitor, a beta-catenin stimulator, a stem cell stimulator, an activator of Lgr5-positive epithelial stem cell proliferation, a cell differentiation modulator, a sensory hair cell regenerating compound, a glutathione peroxidase stimulator, a Vitamin K dependent protein C stimulator, an otoprotectant, a chemoprotectant, a hair cell regenerating compound, a Glycogen synthase kinase-3 inhibitor, a 5-HT 3 receptor antagonist, a calcineurin inhibitor, a free radical scavenger, an anti-inflammatory agent, an apoptosis inhibitor, a neuroprotectant, a cyclin-dependent kinase-2 inhibitor, an apoptotic protease-activating factor 1 inhibitor, a metabotropic glutamate receptor 7 antagonist, a toll-like receptor (TLR) antagonist, a TLR-2 antagonist, a TLR-4 antagonist, a TLR-9 antagonist, a tropomyosin receptor kinase (Trk)-C agonist, a Heat shock protein stimulator, a Guanylate cyclase stimulator, a PDE 5 inhibitor, an antiviral agent, a DNA polymerase inhibitor, a Transferase inhibitor, a KCNC potassium channel 1 modulator, aKCNC potassium channel 2 modulator, and a HSF1 gene stimulator.

8. The method of claim 7, wherein the therapeutic agent is selected from the group consisting of brimapitide, OTO-413, pioglitazone, OTO-510, NXT-596, FX-322, pioglitazone hydrochloride, PIPE-505, otopotin, LY-3056480, ebselen, SPI-3005, ancrod, sodium thiosulfate, ACOU-085, OTO-6XX, DB-020, ORC-13661, FX-345, arazasetron besylate, disufenton sodium, acetylcysteine, AC-102, AZD-5438, LPT-99, NT-12, OR-112, PGT-117, P-13, PIPE-336, OR-102C, ebselen, small heat shock protein, TOP-M119, AP-001, ganciclovir, dendrogenin B, AUT-00206, Dexamethasone (DEX), DEX-salvianolic acid B (DEX-SAL) conjugate, HB-097, and plexaris.

9. The method of claim 3, wherein the therapeutic agent is a cell.

10. The method of claim 9, wherein the cell is a stem cell.

11. The method of claim 10, wherein the stem cell is an embryonic stem cell, ES-like stem cell, fetal stem cell, adult stem cell, pluripotent stem cell, induced pluripotent stem cell, multipotent stem cell, oligopotent stem cell, or unipotent stem cell.

12. The method of claim 4, wherein the vector is a plasmid, cosmid, artificial chromosome, or viral vector.

13. The method of claim 12, wherein the viral vector is an AAV vector.

14. The method of claim 13, wherein the serotype of the AAV vector is selected from the group consisting of AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S.

15. The method of claim 1, wherein the agent is an imaging agent.

16. The method of claim 15, wherein the imaging agent is selected from the group consisting of an organic molecule, fluorophore, metal ion, salt or chelate, particle, peptide, protein, nucleic acid, polymer or liposome.

17. The method of claim 1, further comprising examining a level of the agent in the inner ear after the administering step.

18. A method of treating a subject having or at risk of developing hearing loss, comprising administering an effective amount of a therapeutic agent to the CSF in the subject.

19-36. (canceled)

37. A method of increasing expression of a gene in an inner ear cell of a subject, comprising administering to the CSF in the subject a nucleic acid or a vector comprising the gene.

38. The method of claim 37, wherein the inner ear cell is selected from the group consisting of inner hair cells, outer hair cells, vestibular hair cells, cochlear cells and vestibular supporting cells.

39-51. (canceled)


SEQUENCE LISTING (25 sequences)

SEQ ID NO: 1 - protein, 589 aa, Homo sapiens
MPFKAFDTFKEKILKPGKEGVKNAVGDSLGILQRKIDGTTEEEDNIELNEEGRPVQTSRPSPPLCDCHCCGLPKRYIIAIMSGLGFCISFGIRCNLGVAIVEMVNNSTVYVDGKPEIQTAQFNWDPETVGLIHGSFFWGYIMTQIPGGFISNKFAANRVFGAAIFLTSTLNMFIPSAARVHYGCVMCVRILQGLVEGVTYPACHGMWSKWAPPLERSRLATTSFCGSYAGAVVAMPLAGVLVQYIGWSSVFYIYGMFGIIWYMFWLLQAYECPAAHPTISNEEKTYIETSIGEGANVVSLSKFSTPWKRFFTSLPVYAIIVANFCRSWTFYLLLISQPAYFEEVFGFAISKVGLLSAVPHMVMTIVVPIGGQLADYLRSRQILTTTAVRKIMNCGGFGMEATLLLVVGFSHTKGVAISFLVLAVGFSGFAISGFNVNHLDIAPRYASILMGISNGVGTLSGMVCPLIVGAMTRHKTREEWQNVFLIAALVHYSGVIFYGVFASGEKQEWADPENLSEEKCGIIDQDELAEEIELNHESFASPKKKMSYGATSQNCEVQKKEWKGQRGATLDEEELTSYQNEERNFSTIS

SEQ ID NO: 2 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 3 - protein, 552 aa, Homo sapiens
MDRKVAREFRHKVDFLIENDAEKDYLYDVLRMYHQTMDVAVLVGDLKLVINEPSRLPLFDAIRPLIPLKHQVEYDQLTPRRSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEPLTWQYVDQFVSESGGVRGSLGSPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDRERLAEARQRELQRQELLMQKRLAMESNKILQEQQEMERQRRKEIAQKAAEENERYRKEMEQIVEEEEKFKKQWEEDWGSKEQLLLPKTITAEVHPVPLRKPKYDQGVEPELEPADDLDGGTEEQGEQDFRKYEEGFDPYSMFTPEQIMGKDVRLLRIKKEGSLDLALEGGVDSPIGKVVVSAVYERGAAERHGGIVKGDEIMAINGKIVTDYTLAEAEAALQKAWNQGGDWIDLVVAVCPPKEYDDELTFF

SEQ ID NO: 4 - protein, 530 aa, Homo sapiens [sequence omitted]

SEQ ID NO: 5 - protein, 1675 aa, Homo sapiens
MFRQFYLWTCLASGIILGSLFEICLGQYDDDCKLARGGPPATIVAIDEESRNGTILVDNMLIKGTAGGPDPTIELSLKDNVDYWVLMDPVKQMLFLNSTGRVLDRDPPMNIHSIVVQVQCINKKVGTIIYHEVRIVVRDRNDNSPTFKHESYYATVNELTPVGTTIFTGFSGDNGATDIDDGPNGQIEYVIQYNPDDPTSNDTFEIPLMLTGNIVLRKRLNYEDKTRYFVIIQANDRAQNLNERRTTTTTLTVDVLDGDDLGPMFLPCVLVPNTRDCRPLTYQAAIPELRTPEELNPIIVTPPIQAIDQDRNIQPPSDRPGILYSILVGTPEDYPRFFHMHPRTAELSLLEPVNRDFHQKFDLVIKAEQDNGHPLPAFAGLHIEILDENNQSPYFTMPSYQGYILESAPVGATISDSLNLTSPLRIVALDKDIEDTKDPELHLFLNDYTSVFTVTQTGITRYLTLLQPVDREEQQTYTFSITAFDGVQESEPVIVNIQVMDANDNTPTFPEISYDVYVYTDMRPGDSVIQLTAVDADEGSNGEITYEILVGAQGDFIINKTTGLITIAPGVEMIVGRTYALTVQAADNAPPAERRNSICTVYIEVLPPNNQSPPRFPQLMYSLEISEAMRVGAVLLNLQATDREGDSITYAIENGDPQRVFNLSETTGILTLGKALDRESTDRYILIITASDGRPDGTSTATVNIVVTDVNDNAPVFDPYLPRNLSVVEEEANAFVGQVKATDPDAGINGQVHYSLGNFNNLFRITSNGSIYTAVKLNREVRDYYELVVVATDGAVHPRHSTLTLAIKVLDIDDNSPVFTNSTYTVLVEENLPAGTTILQIEAKDVDLGANVSYRIRSPEVKHFFALHPFTGELSLLRSLDYEAFPDQEASITFLVEAFDIYGTMPPGIATVTVIVKDMNDYPPVFSKRIYKGMVAPDAVKGTPITTVYAEDADPPGLPASRVRYRVDDVQFPYPASIFEVEEDSGRVITRVNLNEEPTTIFKLVVVAFDDGEPVMSSSATVKILVLHPGEIPRFTQEEYRPPPVSELATKGTMVGVISAAAINQSIVYSIVSGNEEDTFGINNITGVIYVNGPLDYETRTSYVLRVQADSLEVVLANLRVPSKSNTAKVYIEIQDENNHPPVFQKKFYIGGVSEDARMFTSVLRVKATDKDTGNYSVMAYRLIIPPIKEGKEGFVVETYTGLIKTAMLFHNMRRSYFKFQVIATDDYGKGLSGKADVLVSVVNQLDMQVIVSNVPPTLVEKKIEDLTEILDRYVQEQIPGAKVVVESIGARRHGDAFSLEDYTKCDLTVYAIDPQTNRAIDRNELFKFLDGKLLDINKDFQPYYGEGGRILEIRTPEAVTSIKKRGESLGYTEGALLALAFIIILCCIPAILVVLVSYRQFKVRQAECTKTARIQAALPAAKPAVPAPAPVAAPPPPPPPPPGAHLYEELGDSSMYEMPQYGSRRRLLPPAGQEEYGEVVGEAEEEYEEEEWARKRMIKLVVDREYETSSTGEDSAPECQRNRLHHPSIHSNINGNIYIAQNGSVVRTRRACLTDNLKVASPVRLGGPFKKLDKLAVTHEENVPLNTLSKGPFSTEKMNARPTLVTFAPCPVGTDNTAVKPLRNRLKSTVEQESMIDSKNIKEALEFHSDHTQSDDEELWMGPWNNLHIPMTKL

SEQ ID NO: 6 - protein, 280 aa, Homo sapiens
MAAMKGHMECVRYLDSIAAKQSSLNPKLVGKLKDKAFREAERRIRECAKLQRRHHERMERRYRRELAERSDTLSFSSLTSSTLSRRLQHLALGSHLPYSQATLHGTARGKTKMQKKLERRKQGGEGTFKVSEDGRKSARSLSGLQLGSDVMFVRQGTYANPKEWGRAPLRDMFLSDEDSVSRATLAAEPAHSEVSTDSGHDSLFTRPGLGTMVFRRNYLSSGLHGLGRTDWAARMGVWMGWERRGVGCRAPPAWTMTAWAVPTACRTAAVGRSCPGMSSI

SEQ ID NO: 7 - protein, 1546 aa, Homo sapiens [sequence omitted]

SEQ ID NO: 8 - protein, 1967 aa, Homo sapiens
MQLCIFCCCCILFYFDLYDFGRGYDFTIQENGLQIDQPPEIGNISIVRIIIMKNDNAEGIIEFDPKYTAFEVEEDVGLIMIPVVRLHGTYGYVTADFISQSSSASPGGVDYILHGSTVTFQHGQNLSFINISIIDDNESEFEEPIEILLTGATGGAVLGRHLVSRIIIAKSDSPFGVIRFLNQSKISIANPNSTMILSLVLERTGGLLGEIQVNWETVGPNSQEALLPQNRDIADPVSGLFYFGEGEGGVRTIILTIYPHEEIEVEETFIIKLHLVKGEAKLDSRAKDVTLTIQEFGDPNGVVQFAPETLSKKTYSEPLALEGPLLITFFVRRVKGTFGEIMVYWELSSEFDITEDFLSTSGFFTIADGESEASFDVHLLPDEVPEIEEDYVIQLVSVEGGAELDLEKSITWFSVYANDDPHGVFALYSDRQSILIGQNLIRSIQINITRLAGTFGDVAVGLRISSDHKEQPIVTENAERQLVVKDGATYKVDVVPIKNQVFLSLGSNFTLQLVTVMLVGGRFYGMPTILQEAKSAVLPVSEKAANSQVGFESTAFQLMNITAGTSHVMISRRGTYGALSVAWTTGYAPGLEIPEFIVVGNMTPTLGSLSFSHGEQRKGVFLWTFPSPGWPEAFVLHLSGVQSSAPGGAQLRSGFIVAEIEPMGVFQFSTSSRNIIVSEDTQMIRLHVQRLFGFHSDLIKVSYQTTAGSAKPLEDFEPVQNGELFFQKFQTEVDFEITIINDQLSEIEEFFYINLTSVEIRGLQKFDVNWSPRLNLDFSVAVITILDNDDLAGMDISFPETTVAVAVDTTLIPVETESTTYLSTSKTTTILQPTNVVAIVTEATGVSAIPEKLVTLHGTPAVSEKPDVATVTANVSIHGTFSLGPSIVYIEEEMKNGTFNTAEVLIRRTGGFTGNVSITVKTFGERCAQMEPNALPFRGIYGISNLTWAVEEEDFEEQTLTLIFLDGERERKVSVQILDDDEPEGQEFFYVFLTNPQGGAQIVEGKDDTGFAAFAMVIITGSDLHNGIIGFSEESQSGLELREGAVMRRLHLIVTRQPNRAFEDVKVFWRVTLNKTVVVLQKDGVNLMEELQSVSGTTTCTMGQTKCFISIELKPEKVPQVEVYFFVELYEATAGAAINNSARFAQIKILESDESQSLVYFSVGSRLAVAHKKATLISLQVARDSGTGLMMSVNFSTQELRSAETIGRTIISPAISGKDFVITEGTLVFEPGQRSTVLDVILTPETGSLNSFPKRFQIVLFDPKGGARIDKVYGTANITLVSDADSQAIWGLADQLHQPVNDDILNRVLHTISMKVATENTDEQLSAMMHLIEKITTEGKIQAFSVASRTLFYEILCSLINPKRKDTRGFSHFAEVTENFAFSLLTNVTCGSPGEKSKTILDSCPYLSILALHWYPQQINGHKFEGKEGDYIRIPERLLDVQDAEIMAGKSTCKLVQFTEYSSQQWFISGNNLPTLKNKVLSLSVKGQSSQLLTNDNEVLYRIYAAEPRIIPQTSLCLLWNQAAASWLSDSQFCKVIEETADYVECACSHMSVYAVYARTDNLSSYNEAFFTSGFICISGLCLAVLSHIFCARYSMFAAKLLTHMMAASLGTQILFLASAYASPQLAEESCSAMAAVTHYLYLCQFSWMLIQSVNFWYVLVMNDEHTERRYLLFFLLSWGLPAFVVILLIVILKGIYHQSMSQIYGLIHGDLCFIPNVYAALFTAALVPLTCLVVVFVVFIHAYQVKPQWKAYDDVFRGRTNAAEIPLILYLFALISVTWLWGGLHMAYRHFWMLVLFVIFNSLQGLYVFMVYFILHNQMCCPMKASYTVEMNGHPGPSTAFFTPGSGMPPAGGEISKSTQNLIGAMEEVPPDWERASFQQGSQASPDLKPSPQNGATFPSSGGYGQGSLIADEESQEFDDLIFALKTGAGLSVSDNESGQGSQEGGTLTDSQIVELRRIPIADTHL

SEQ ID NO: 9 - protein, 907 aa, Homo sapiens
MNAPLDGLSVSSSSTGSLGSAAGAGGGGGAGLRLLSANVRQLHQALTALLSEAEREQFTHCLNAYHARRNVFDLVRTLRVLLDSPVKRRLLPMLRLVIPRSDQLLFDQYTAEGLYLPATTPYRQPAWGGPDSAGPGEVRLVSLRRAKAHEGLGFSIRGGSEHGVGIYVSLVEPGSLAEKEGLRVGDQILRVNDKSLARVTHAEAVKALKGSKKLVLSVYSAGRIPGGYVTNHIYTWVDPQGRSISPPSGLPQPHGGALRQQEGDRRSTLHLLQGGDEKKVNLVLGDGRSLGLTIRGGAEYGLGIYITGVDPGSEAEGSGLKVGDQILEVNGRSFLNILHDEAVRLLKSSRHLILTVKDVGRLPHARTTVDETKWIASSRIRETMANSAGFLGDLTTEGINKPGFYKGPAGSQVTLSSLGNQTRVLLEEQARHLLNEQEHATMAYYLDEYRGGSVSVEALVMALFKLLNTHAKFSLLSEVRGTISPQDLERFDHLVLRREIESMKARQPPGPGAGDTYSMVSYSDTGSSTGSHGTSTTVSSARNTLDLEETGEAVQGNINALPDVSVDDVRSTSQGLSSFKPLPRPPPLAQGNDLPLGQPRKLGREDLQPPSSMPSCSGTVFSAPQNRSPPAGTAPTPGTSSAQDLPSSPIYASVSPANPSSKRPLDAHLALVNQHPIGPFPRVQSPPHLKSPSAEATVAGGCLLPPSPSGHPDQTGTNQHFVMVEVHRPDSEPDVNEVRALPQTRTASTLSQLSDSGQTLSEDSGVDAGEAEASAPGRGRQSVSTKSRSSKELPRNERPTDGANKPPGLLEPTSTLVRVKKSAATLGIAIEGGANTRQPLPRIVTIQRGGSAHNCGQLKVGHVILEVNGLTLRGKEHREAARIIAEAFKTKDRDYIDFLVTEFNVML

SEQ ID NO: 10 - protein, 120 aa, Homo sapiens
MQALQQQPVFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWLTKGHS

SEQ ID NO: 11 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 12 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 13 - protein, 261 aa, Homo sapiens
MDWGTLHTFIGGVNKHSTSIGKVWITVIFIFRVMILVVAAQEVWGDEQEDFVCNTLQPGCKNVCYDHFFPVSHIRLWALQLIFVSTPALLVAMHVAYYRHETTRKFRRGEKRNDFKDIEDIKKQKVRIEGSLWWTYTSSIFFRIIFEAAFMYVFYFLYNGYHLPWVLKCGIDPCPNLVDCFISRPTEKTVFTIFMISASVICMLLNVAELCYLLLKVCFRRSKRAQTQKNHPNHALKESKQNEMNELISDSGQNAITGFPS

SEQ ID NO: 14 - protein, 279 aa, Homo sapiens
MCGRFLRRLLAEESRRSTPVGRLLLPVLLGFRLVLLAASGPGVYGDEQSEFVCHTQQPGCKAACFDAFHPLSPLRFWVFQVILVAVPSALYMGFTLYHVIWHWELSGKGKEEETLIQGREGNTDVPGAGSLRLLWAYVAQLGARLVLEGAALGLQYHLYGFQMPSSFACRREPCLGSITCNLSRPSEKTIFLKTMFGVSGFCLLFTFLELVLLGLGRWWRTWKHKSSSSKYFLTSESTRRHKKATDSLPVVETKEQFQEAVPGRSLAQEKQRPVGPRDA

SEQ ID NO: 15 - protein, 270 aa, Homo sapiens
MDWKTLQALLSGVNKYSTAFGRIWLSVVFVFRVLVYVVAAERVWGDEQKDFDCNTKQPGCTNVCYDNYFPISNIRLWALQLIFVTCPSLLVILHVAYREERERRHRQKHGDQCAKLYDNAGKKHGGLWWTYLFSLIFKLIIEFLFLYLLHTLWHGFNMPRLVQCANVAPCPNIVDCYIARPTEKKIFTYFMVGASAVCIVLTICELCYLICHRVLRGLHKDKPRGGCSPSSSASRASTCRCHHKLVEAGEVDPDPGNNKLQASAPNLTPI

SEQ ID NO: 16 - DNA, 3838 bp, Homo sapiens
  source 1..3838: mol_type = unassigned DNA
tcttggaagatccgagctgggtttcatctcctttttgattttgagtagttccctccacgagaactgacttccaggtgttcaccaagggaaacaaggtggttctcacactggaaatgaggaaggatgacagtttttgagactgactgttaacggctcagaggtgcccctcattcaaaatgccttttaaagcatttgataccttcaaagaaaaaattctgaaacctgggaaggaaggagtgaagaacgccgtgggagattctttgggaattttacaaagaaaaatcgatgggacaactgaggaagaagataacattgagctgaatgaagaaggaaggccggtgcagacgtccaggccaagccccccactctgcgactgccactgctgcggcctccccaagcgttacatcattgctatcatgagtgggctgggattctgcatttcctttgggatccggtgcaatcttggagttgccattgtggaaatggtcaacaatagcaccgtatatgttgatggaaaaccggaaattcagacagcacagtttaactgggatccagaaacagtgggccttatccatggatcttttttctggggctatattatgacacaaattccaggtggtttcatttcaaacaagtttgctgctaacagggtctttggagctgccatcttcttaacatcgactctgaacatgtttattccctctgcagccagagtgcattacggatgcgtcatgtgtgtcagaattctgcaaggtttagtggagggtgtgacctacccagcctgccatgggatgtggagtaagtgggcaccacctttggagagaagccgactggccacaacctctttttgtggttcctatgcaggggcagtggttgccatgcccctggctggggtgttggtgcagtacattggatggtcctctgtcttttatatttatggcatgtttgggattatttggtacatgttttggctgttgcaggcctatgagtgcccagcagctcatccaacaatatccaatgaggagaagacctatatagagacaagcataggagagggggccaacgtggttagtctaagtaaatttagtaccccatggaaaagatttttcacatctttgccggtttacgcaatcattgtggcaaatttttgcagaagctggaccttttatttgctcctcataagtcagcctgcttattttgaagaggtctttggatttgcaataagtaaggtgggtctcttgtcagcagtcccacacatggttatgacaatcgttgtacctattggaggacaattggctgattatttaagaagcagacaaattttaaccacaactgctgtcagaaaaatcatgaactgtggaggttttggcatggaggcaaccttactcctggtggttggcttttcgcataccaaaggggtggctatctcctttctggtacttgctgtaggatttagtggcttcgctatttcaggttttaatgtcaaccacctggacattgccccacgctatgccagcattctcatggggatctcaaacggagtgggaaccctctctggaatggtctgtcccctcattgtcggtgcaatgaccaggcacaagacccgtgaagaatggcagaatgtgttcctcatagctgccctggtgcattacagtggtgtgatcttctatggggtctttgcttctggggagaaacaggagtgggctgacccagagaatctctctgaggagaaatgtggaatcattgaccaggacgaattagctgaggagatagaactcaaccatgagagttttgcgagtcccaaaaagaagatgtcttatggagccacctcccagaattgtgaagtccagaagaaggaatggaaaggacagagaggagcgacccttgatgaggaagagctgacatcctaccagaatgaagagagaaacttctcaactatatcctaatgtctgagaggcacttctgtcttctccttactttagaaacagaaagtatccatacctattgcctttcttgtagcccagcttgccagaggtccaaatattgggaggggagaagatctaaccagcaacagggaaaagagaaatattatctttcaatgacatgtataggtaaggagctgcgctcagttgataacatagttgataatacatattttttgaattgacagttgacccttctctcaaagagctaaacttattcagaaaggaatgactagaagaaaaaggagacaataccatgttgttcaaagaaacattgaaggaaattgggatgtttggccagaaggaatgtaaacagtagtagtagctgccaccacatctctagggtagccatgcagaggagggcttcatattcccaataaaccccacgttgtggcaggtgctttataaacactcttatttaatctccacacctttatgacacacatttcttatccccattttacaaccaaggcatctaaagcaacaagaaatgaacttgcccaaggtcatctgccagggtcagtgctgagactgttgaagctctcaataggtggcagttttagggaagatttccattcagtgtagggaagacatttgtaataatgaaaactgaaaatggagtaattgtgagtaactcaccactttagcaggtgttggggaagggaaacatttgggttgatgaggcagaggggattcaaatgtgtgagaggctagattcaaagaccctcagtgttctatgttatctgaagagtcaaatggttttgtgactccatagtttttaaagtaataagggtcaaagactacatcagagattcaaataggtttttaaagaaaagctaagcaagagagccaaatttttagaaatctgatggtcaaaatagctgaaagcagtaaacaagagattggctattaaatttcaactttccataatattaagaatgtagctaaatgatgtcccaaactacttacaaacttttaagacatttaataatttaagaagtaggttcatgtgttttcttaggtaaagttcttctgaaagaattttctatttttaaaaaatgtatctctttagccttttctgctggagattatattaggaagtttcatcagattgtataaaattatgattttgtatcaaaagtattcatgatgactctatttggaatgatattcagggaaatcacaataatatagcagtagttatacagagaaatactacaatgaaaacatttggggcaattagacctacagttactgttgaaaaattcacctttgattgcataaggcaattacatggatacttttagatatatttaaaattttaacattggcatctaaagtgttatttgaaaataaaattattttcctgttcattgattttaaacattttattcctactttcagaagaaaaatataatacggaaaaaattatagatttacttgtagcttattattgtaaagtgttttttttttttttctaatttctcccacatgtatttctggtccccagtgatactagctgagttgtagtgtattttataaatggaataatcttggggaaaaattgcgattcttcattaaataatattctttatgtcactagcatacaatttatgttagtagacatctttaaatctctttaatgagtgaatccatgcaagccccataaaacagttcctagcatgcagaaaatgcccacgtaaatagctgtcatcatcattatcttttaacattttgggggactttccagttgaaaagaaaacatgctatgtcatttttatccattatccctggaacttattgtgaaagttgtgctgttttctaagtaaaataaaaaataaaaaattagcaattta

SEQ ID NO: 17 - DNA, 2091 bp, synthetic construct
  source 1..2091: mol_type = other DNA
gactttcctgtgtccctctgtccccctggggggaagggacaatggaggaatattccccaggcctgggcgactgtccgctggtcagagggaaggccccgctgcctgcccggtgcacctgtgagctgagattgtggggatcattcagtcattcctttattcagtagatctgtacggggctggctttgtgccaaggcctgccccgggcacctgggatgtggggaaccaggcatagcaatccctgctctgggacagtctacaaacgagcagcgagaacaaagacatactctggtcatgacagacactaccccgcacattactgatggggcagtgtgggcctgctgggttagagacggcctactccctgatctgtgcatgaagggctgggaagaggagatgggccttcggggcacaggaaggtgtggggggaccagagcggaggctgctgagggcagatgcaggcatctagggccaggaagctggtgtggtggtggggagaagcagggttgggatgactgaaagcagagtggacgactcaatgaaaaacgacttgggagacggggtgagtgcagtgtgagggcaaagtgaaccgagatgcctcgctttcttcatgggactgagaaatgggtattgcaaatcgtgtgatctgcttagcatagtgcctggttcatagcatggctgaacacaatgtagctgtcatcctggtgtttggaggccgccctgtcccccataacctgcccacctctctgccccattcccacagctcactgcattttgtccctgctgccagtcgtgtgccatgggctctcctgtgctcccccggggtctcatttccccatccgtgtagggctgacgccactggccccgcccagcctgggaggatggagaggggggacttgaggagtggccagtactgggtttcggcagcaggtgcaaatcccaggtggggactgtgttccagggactcctggactcctgttaggccagcaggctaaggggcagacggcatcctggggtcttcctgggacagggtcgtcgggtctgaggggacccaggccctagcagaagctgactcctgtcacggtcacttcccagcggtgctcagcccgcctgacggaagcagctgggcagtggggcctgtgaccagcaggcggcgctggcgagctatagcgcccctttgggcccccaccccaccttctgggcagcttccctgcagacaccccagttatgggggctagggacccaaaagagacatccttctgccacccagagctgccctggcgaggtgcactatggggccgccgacagctgcgtggctgccgagggcggaaaggagaaactgtcatgtcccgatagggccgcgcgaggtctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagaccccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagca

SEQ ID NO: 18 - DNA, 2100 bp, synthetic construct
  source 1..2100: mol_type = other DNA
gtaaggatgggctgcccactgtcctgggcattgggaggggtttggatgtggaggagtcatggacttgagctacctctagagcctctgccccacagccacttgctcctgggactgggcttcctgccacccttgagggctcagccaccacagccactgaatgaaactgtcccgagcctgggaagatggatgtgtgtcccctggaggagggaagagccaaggagcatgttgtccatcgaatcttctctgagctggggctggggttagtggcatcctggggccaggggaatagacatgctgtggtggcagagagaagagtccgttctctctgtctcctttgctttctctctgacactctttatctccgtttttggataagtcacttccttcctctatgccccaaatatcccatctgtgaaatgggagtatgaagccccaacagccagggttgtagtggggaagaggtaaaatcaggtatagacatagaaatacaaatacagtctatgccccctgttgtcagttggaaaagaaattaacttgaaggtggtctagttctcatttttagaaatgaaatgtctgtctggtcattttaaaatgtggcccttaaatttcacgccctcaccactctcccccatcccttggagccccatgtctctagtgaaagcactggctctgcccccagccctcatggctcatgctggcatagggcgcctgctccacagcctgggcaccatcttcagacaagtgcccggtggcaactgcctgctggccctgttgaatccacatctccaccaggcatccagactagttcaggtctctggaaggactgtgggtttgctgtgtcccagagctccagggcaggggtcagggctcggatgtcgggcagtgtcatgggcagaggatcgaatgccccggcggctctgaatgggcccttgtgaaaaattgatgcgcattctaggagacaggttgggagccagaggggcctcataccagggtctgtaggctggggctgccttttaagctccttcctgaggccgtctctgggtctggccctgtgctggacaaggctggagacaaggcaatgtctcagaccctctcccattggccacatcctgccctggatcaactcgccaactttgggggcagaggtgggactgacccttaccctgacaacataatgcatatagtcaaaatgggataaaggggaatatagaggctcttggcagcttgggagtggtcagggaaggcttcctggaggaggtatcatctgaactgagccatgaaccataagtggaaattcactagtcaaaatttcaggtagaagggccagtgtgtgaaggccaggagatggcaagagctggcgtatttcaggaacagtgagtcactgaggatgtccaagtataagggtaggaaagggagtgagcagtgagagaaaagaccgaggcatcagcaggggccagattgtgctgggcctagcggggcgggcccgggcccgggcccaggcccaggtgcggtgcattcagcagcacagccagagacagaccccaatgaccctgcctccccgtcagcagccagtgctctgcacagagccatcctgagggcagtgggtgctcttgagaggtttcaggcagggtgtgctgtgagcaggtcatgcccagcccttgaccttctgctcagtcaggcttgtccttgtcacccacattcctggggcagtccctaagctgagtgccggagattaagtcctagtcctaaatttgctctggctagctgtgtgaccctgggcaagtcttggtccctctctgggcccctttgccgtaggtccctggtggggccagacttgctactttctaggagccctttgggaatctctgaatgacagtggctgagagaagaattcagctgctctgggcagtggtgctggtgacagtggctgaggctcaggtcacacaggctgggcagtggtcagagggagagaagccaaggagggttcccttgagggaggaggagctggggctttgggaggagcccaggtgaccccagccaggctcaaggcttccagggctggcctgcccagaagcatgacatggtctctctccctgcaga

SEQ ID NO: 19 - DNA, 2046 bp, synthetic construct
  source 1..2046: mol_type = other DNA
ttctttcttgacagtttttttattctttcatcattttaaatatgccatcctactgccctctggccgccgtggtttctgatgagaaatcagccgagactcatactaacgattcgttgtatagaatgagtcacctctctgttgctgctttcaagagtctctgtctttgccacttgtggagaagaggcctattcctccagctcaagggtggtctcagtagtcgttctggaaccaggcaaggtcaggttcttagcaccttgcagggtctcccccaggggacctctctttagccaccagccgccagacttactttacaatctcttccctctctcctctgattctctgagtttgctgttccctcttcctgggatgccctttcccctaatgtctgcctaaaaaaaaaatcttaaaacccagctgtcacccctaacacgactcccttagttaaccatggctggcactcatatacatttggtctaacaggcaccaatctaggtgcattacgcatcttaacctgttaaatcctcacgagcacgtggaagcagtgtcattttgtcacctgacggatgaggaaagaaaggaacatagaggttgaggaacttgcccaaggtaacacagcttgtgagtggctgagctgggattgaaactaagccatctgactccagggtccaaactcttagtggccatgctatcctatgtctccgggactgatctgcctcttatctggtggtaccatccttatctgtgcctgggtctgtccaccacaccaggctgggagctctgaggacatgtcttgttaacagtgtcctctgtactttagtctgaacctgtactcagtgaaggtcggttatatgaactcgtggtagaatgcctggaagctcaccaccagcacttccttgtgtctgaggtctgggtcctgtcccaatgtactgctatagatgggcggggagagataggtgtgcctgttttcggcttgactgggggaaggacctctcctgagagcggtgtccctgagaggtcacaggaagtgagtgggaatcactgatcagtcagcttcgtgctggacagcagagccctgggttcccaaggctgggacagggagtggggtagaactgggcccatcctgggtggctctgagccctgtgtgaggcatcaccgtctttttcgttgccattccccccacccagtcccccgcccccactctctgccgttgttcattcgttggggagcagttggcttccaggatactaggcaatgcagagagccacaaatgcagactggaggccttgaaaagagaagaggaagaaaaaggaggaaagaggggaggaggacaaagataggcagggcaaggcgagaaggagggaatgggagaggagtcacagggcctgggccttccggaccctcgggatgggacggtggcctctggctcacagcccagcgtggaccgacgcctggtgtccagccctgtgtggctgctaggacacaggaggccagcttggcccattgcagggagagctctgagccatcgaggtggttcagctgtcctggggagttcaggggaccccacaccaggcctccaaggcctcttctgcaggatggacgtgggctgctatttgacctcgggggctgcgggaggaggacatagcctcttcaggggctttcccccaaacttaccccagaaggaaactgtatctgccctcactccccagaggcagccagggagccagaaatatcttcaatgcatctctcctttagtcttaaaaaccccgggagtttaccgcgttctagtccataacatcctcgtgtttccttccatggaaaacatagaaaaaaacatgacgattttttccaaaactgccgtcaggtgcggtgccaggtgccacgtttatcccgggcctaccgccgcggtgccagtgaagccgcctgggattccgggcaaagtggggctgtggggggttcggccgagacggagggggccggggtggcgcgggcgccccctcgccgtcccgcccgcgggcgtcacctaagccgccgttgccatgggcccgc

SEQ ID NO: 20 - DNA, 3221 bp, synthetic construct
  source 1..3221: mol_type = other DNA
agacaccccagttatgggggctagggacccaaaagagacatccttctgccacccagagctgccctggcgaggtgcactatggggccgccgacagctgcgtggctgccgagggcggaaaggagaaactgtcatgtcccgatagggccgcgcgaggtctccatcctcgacaacgctaataacaaagacgtgtgctcctctttgcttggttccccccactcctttaaatcacagatttcacttcagtttatctgtgtcgctgtcacacgtggggtggctcccagtcagctggtttggcaaagtttctggatgattacggaataacatgtgtccccaacccgcagagcaggttgtgggggcaatgttgcattgaccagcgtcagagaacacacatcagaggcaagggtgggtgtgcaggagggagaaggcgcagaaggcagggctttagctcagcactctccctcctgccatgctctgcctgaccgttccctctctgagtcccaaacagccaggtagaggaggaagaaatggggctgagaccccagcacatcagtgattaagtcaggatcaggtgcggtttcctgctcaggtgctgagacagcaggcggtgtcctgcaaacaacaggaggcacctgaagctagcctggggggcccacgcccaggtgcggtgcattcagcagcacagccagagacagaccccaatgaccccgcctccctgtcggcagccagtgctctgcacagagccctgagcagcctctggacattagtcccagccccagcacggcccgtcccccacgctgatgtcaccgcacccagaccttggaggccccctccggctccgcctcctgggagaaggctctggagtgaggaggggagggcagcagtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccaggtaaggatgggctgcccactgtcctgggcattgggaggggtttggatgtggaggagtcatggacttgagctacctctagagcctctgccccacagccacttgctcctgggactgggcttcctgccacccttgagggctcagccaccacagccactgaatgaaactgtcccgagcctgggaagatggatgtgtgtcccctggaggagggaagagccaaggagcatgttgtccatcgaatcttctctgagctggggctggggttagtggcatcctggggccaggggaatagacatgctgtggtggcagagagaagagtccgttctctctgtctcctttgctttctctctgacactctttatctccgtttttggataagtcacttccttcctctatgccccaaatatcccatctgtgaaatgggagtatgaagccccaacagccagggttgtagtggggaagaggtaaaatcaggtatagacatagaaatacaaatacagtctatgccccctgttgtcagttggaaaagaaattaacttgaaggtggtctagttctcatttttagaaatgaaatgtctgtctggtcattttaaaatgtggcccttaaatttcacgccctcaccgctctcccccatcccttggagccccatgtctctagtgaaagcactggctctgcccccagccctcatggctcatgctggcatagggcgcctgctccacagcctgggcaccatcttcagacaagtgcccggtggcaactgcctgctggccctgttgaatccacatctccaccaggcatccagactagttcaggtctctggaaggaccgtgggtttgctgtgtcccagagctccagggcaggggtcagggctcggatgtcgggcagtgtcatgggcagaggatcgaatgccccggcggctctgaatgggcccttgtgaaaaattgatgcgcattctaggagacaggttgggagccagaggggcctcataccagggtctgtaggctggggctgccttttaagctccttcctgaggccgtctctgggtctggccctgtgctggacaaggctggagacaaggcaatgtctcagaccctctcccattggccacatcctgccctggatcaactcgccaactttgggggcagaggtgggactgacccttaccctgacaacataatgcatatagtcaaaatgggataaaggggaatatagaggctcttggcagcttgggagtggtcagggaaggcttcctggaggaggtatcatctgaactgagccatgaaccataagtggaaattcactagtcaaaatttcaggtagaagggccagtgtgtgaaggccaggagatggcaagagctggcgtatttcaggaacagtgagtcactgaggatgtccaagtataagggtaggaaagggagtgagcagtgagagaaaagaccgaggcatcagcaggggccagattgtgctgggcctagcggggcgggcccgggcccgggcccaggcccaggtgcggtgcattcagcagcacagccagagacagaccccaatgaccctgcctccccgtcagcagccagtgctctgcacagagccatcctgagggcagtgggtgctcttgagaggtttcaggcagggtgtgctgtgagcaggtcatgcccagcccttgaccttctgctcagtcaggcttgtccttgtcacccacattcctggggcagtccctaagctgagtgccggagattaagtcctagtcctaaatttgctctggctagctgtgtgaccctgggcaagtcttggtccctctctgggcccctttgccgtaggtccctggtggggccagacttgctactttctaggagccctttgggaatctctgaatgacagtggctgagagaagaattcagctgctctgggcagtggtgctggtgacagtggctgaggctcaggtcacacaggctgggcagtggtcagagggagagaagccaaggagggttcccttgagggaggaggagctggggctttgggaggagcccaggtgaccccagccaggctcaaggcttccagggctggcctgcccagaagcatgacatggtctctctccctgca

SEQ ID NO: 21 - DNA, 2032 bp, synthetic construct
  source 1..2032: mol_type = other DNA
aaagttctacttaagcatctacagctactagcaattattttctcaagaagcacattttatatggtagcactatttatagtaacagcattattagtcgatttcaataacacccacttcaatcaccaaacggagaattttctgcccaaaaaaaaaaaaatacattagatggccttttctttttctttgttctcatggatggttttcagattaggtttgttttttgttgttgcttttgtgtgtctttaacatttatttcagcagagcccaaacattcacacaagaataaagctctgaagtttctggcacgtaagatacgcccaagactattcctagacaccagcaaacagacgaggttgaagaagaaacaggtgaagaataatgtcttaaacatcattttagcaccctattctgccattctgtatctttataggatatgtccagtccagtcccagaaaatctactgcccaaggagttctagatgacaggttagatcaagccccggcaaatgtttgcgaaaacaacattcaactgcctctggacattgcaaagacatattccagggagctatctcttgggctcttttttaacatctctcctagaggatgagactcctttcgggattacagccactgggaaagtaaaagcaacagtagcatcaacaggttgcatcgcactcccgcgccccaggggcggtgcaggagagcgcccggagccagagccagggcgcgctgggcgcaagctggggcgcgcccagggccaatgagcttctgattggctctttgctgtgaatagactaggccgaggctaataggaccggagggggcgttcacctgggaattggcctcccccccttgccaaggacctccctgattgtatggggcggagtggggtgggggtgggggctggaaaggggaagcccaccctaactaaacggaggggcgggaggaggttaaactgaggaagccacgagcccgcagtaaagagagggcgatcgaggcaaaagggtaggtaacttgggacttctgtgccttgaaagtgttaaggattgcagggaatgcaggcgcctttcattttactttgggcaccctcccctggctgggcatttaatgaggagtcctactgtgtgtgttgaaactctgcataataccgtaaaatattctggccctaaattacactcaacggagagatttaggcagattacaatcctaattggccgcagggtgctggggaaaggggacagagagaattgggggatctggaatttggtgtgctatcactacaccgcagctctattttccattaagaaaaagatcatatgacgaagtcgaactaagagatctgaagtaaaaaatgaaaagatggagcaaaagtaagaaacatactctgagacgagtgggttttccccctttattccaacagcaatcttaaatgatggacgtcatgtagcagttatatatctatgaacatgcatgagagatttataaatacctgcatacataaatacaaacatcctattatacatgagaaatcgtaaatgcttgggcatcagaagtgggagctgtgatcctagcttgggggcagcacagggtaggcggccttctctctgctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaacc

SEQ ID NO: 22 - DNA, 1397 bp, synthetic construct
  source 1..1397: mol_type = other DNA
tccaaggtccggcaatgaagtttgcataacaaacgtttggcagctccctctctcacaccccattaacaagctgtaacatatagctgcaggttgctataatctcattaatattttggaaacttgaatattgagtatttctgagcgctcattccccatatgccagaccactcctgccatgctgactggttcctttctctccattattagcaattagcttctaccttccaaagtcagatccaagtatctaagatactaccaaaggcatcaactatgtatgcaagttaggcatgcttaatatcacccaaacaaacaaagagtcagcacttcttaaagtaatgaagatagataaatcgggttagttctttgggacaccgctgttgttttccagagtttttctatactttaagcagcttgttttatattctgtctttgccctcagccagctaacattttatttgttgagggttttggctcaccacacttttggaaacttatttgatttcacggggagctgaaggaagattgtttttggcaacaggcaagtttaacacgttcttcatggggcattgcgaatggcacatctaccagaaagggagggggagtaacttcctcgtgctgaaccagcaggagaccagagctttcctgaggtcttcctattgattttaaagatttaaaactgagccccaaagttgtaatgttattgaagtttgtcttggaatatacatctcctctgctaacttaaaagttcaagaaaggaaaggaaagaaatagaaccccttgctaactacaacctagactgagaggtgaagatcgcgggcaaagacaggtggtcactgaaacgtttgcagttcttttcttccgaaggcttaggacacagggtaaggaggagctaaaataaagccgagtgtacgtttagtcttctctgcaccccaggcctagtgtctccccaggcaaggagtcaccccctttgcttctggctcctaactgaaaaaggcaaaagggagtggagaatgggttaaatcccaggacacaggggagaggcaggggaggagagaagtcggaggaagataaaggaaaggacaggaaccaagaagcgtgggggtagtttgccgtaatgtgagtgtttcttaattagagagcggctgacaatagaggggctggcagaggctcctggccccggtgcggagcgtctggagcggagcacgcgctgtcagctggtgagcgcactcgctttcaggccgctccccggggagctgagcggccacatttaacaccgtcgtcaccctccccggcctcctcaacatcggcctcctcctcgtagacagccttgctcggccccccaccggcagagtttacagaagccagagcctctcgccgttcccccgcattcgcccggg

SEQ ID NO: 23 - DNA, 6272 bp, synthetic construct
  source 1..6272: mol_type = other DNA
ggcaattggggaggaggaaggcattgggagaaaacaacccgaaggaaaaagaagctggctctttgcaatgggagaaggggttcactcctgaactaggggcaggcctgcgcctgggcggacgctgtagggtgggaacgctcgataactggagagtcccagagaccaccaggcctttccgcaccaggaaaaaaaaaaaaaatctgctctgagaatcttcatctcggtccaccagggtgagtttgggcctggaaaatgggggaaagaaaagagagtagcgaaaggttttcgggttgcggcgccccgcgtgcagacgtctgctctccccagccgcccagcctgctgtggcagccacgctgtgcgcgtgtctttatccttcagttcaaaccagcccccacatcttgaagtaatcagcagccgagaagagacttgtccccctggcatctcaggttaaagaaggaggtccaggaaaatgtggtagagccattacaagaaacccgagccgcagtcatcagggactccaattgccatcgcattaggattgattagagaccagcagtaccgtaatgacccggaaagaggggcagccggggattattaatatcagaatgcgggggtggggggggagtgggtgggcaaagtgggtagggagaagtttcaaattactaggatttatatgtaagtgcaggtttacctgaggaaaccagatagtcagtgaatcacgctaacgataaatctaattgcatttcaaatttcacgaagcctgatttttttggagagtggagtaaaaccatcatgaaaccaagccaatatcttagtttttataaacacatagttttataagcttattgtaagaattcaagtgaagagatggggaggggtgctaaatgtctgctttccttgtcactgggcctccttaccatttggactccaaatagcttgatttttcttctcgtgggagtacatttttgagtcgctttggttcttccctccagggatatggggcaatggagtggacattttttcagagcctgcagaggagtaataaaccaaaatttaagtgaagcagtgatacctgggtagtggtttttgttttttgttttgttgaagacatgaatgatcaggatcccaaggagatgtggagaagagtggtttgtctccttcctccaaatatgagccaaaacaaatcagcccctgatggtctgggctgaaatgcagctcactggataattaactccatctttccagtccaccctaactgtttatttagtgtcaccctgcattaccgtcactagcactttcctctgaacaggaggagctgcaggctttcacctcccaaaaggaaattctttttagcttagacagatgtggtattcaggatgtacataagttgagaaaaaaaagttgattggaaattcaatggaagatagcggttttggagagtttcatttcataaacctgccctaaggagacattttaaaaaacagggaatggggggtggggcgcagagtccctttaattcttaagaattgtttcttgtgacaccttggctccactttccagagaggtagttggcatctgatgttgagtagggtcttctagctcaaacctggcgattggtcagagctttctcagagggcaatttgttaacccttttctcccctaggtggccaatacacctttgcccgccctccgtgcaggctacaccatctattgcccgccctccactccctgtgaaaggttctcgccaagtaaggcctactacaccgcccagacttgagggtctactttcaagagaacttacctgcgctgcctcagtgggtgacataagcacactctgtgaaacctcacagcgcccagtcccggagagaaaaaaacgagcgcccagagccgaggggaagggagagggagggaaggaggaagaaagaggaggatgagaagaggaagaggaaaaacaatgagaaagaagagagagagataaggagagagaatgagaaacaggttcacatacagacagaatgaacgagcgtcctctttcagatggagaaaagagttagagacacactctgggaaagacctgccacggggacacacgtagagggagagagagaataactcttccaaagacacacaccccagctctgttcccctccagtctgagctccctcccagtgcttgaccctcagtcggtccatcagtctgtgtgtgtttctctctttctcccttccctgacctcgagacaatacggagatggtattttcgggagccgcgcggggcggcagggttttcagcggcgagccaggctcacagctggctctgtttgtccaggtgtgggcggtggaggtggtggggggcgtcagtcgccctgaatcttgggccagcgcagcgttttcgcaccaaagaagtaggaggctctgctctcgagcttcccgcttccaggagaaaacttcctccctccatacatacataaatacacttggggaagagacaaatcttcattgttcagaaataagctgaggaggcccgcctgctcccctcagccaggcagagcgaggcagggagggaggcgggggccgcgcggatccggtgtcacgttgcagattcccactaaagataacgaaacgtgatctatttttcagaacagcaagtggatgggaaagaaccttgacaacacaaaactcatttactaatggactggttttgggtttctcttctctgagtccgcaaatctctctgcctgagccccaggcctattcagacctcgcggggagcggcgtcccccaggagcaggtgggctgctgctccggcgccacgccggccatcggagccggagttcccaagggtcgagggcacccggagtttccgactaagcagaggaaaaagaattttggcacttcagccggatttggggagtctccgcactgccccagagcgatgacacgaatttattgaagaaacacctctctatgccctccctttccaaatatttgtaggtagagtagaatcctcaccgtcgttctggatggtgaaagaattccaagaaagcggcccgggagcccctaactatcttgctacaagccagaagggaaacaagtgggtcccagctaggggtgtttgcatcaaggcctgtcaacccagccgagaaaccctagcaacagctaatgctgtcccactgaatgctttttcatcgactaaagcgcacgcggccgttacctgcacagaggaggcactggagagccgaaacctggcatcgggcgctccttgccagcggtgttccctcccgagccgcgctgcagcgagggcagcagccggctggctagagttgctcatttcctttaaatctccaacgcatctctgcttttcgtgcgatttgcattgacagcaaaggttttatgttacgagaacaactcaaacctttctgtgtgcgatttaaaatggatcatttagtttaggtggtttgtaaattcatgtcattttacctctttcagcggttttcagtgaaaggttccattttgggaaaccttatatatccgagccaataattaacatttataacaattggttcccctaaccgagggatcgaaatagcaggtagactccttggtataaagcgcatagcactttccctttccagttacgcgatctattggattaagcgggaggaaaaatgttggccggcccagtcacggcctccgaggaagagcacctcttttctgcccgggttcccgcaatcacaccttctacacgtcgtcttcccagatgacggcaatgtagtacgtcctggttccttttgaagacagataaaaagaaatgggcaggtaggtctggaggaagccgggaaaaaaaggtggtcctatttgtgtctaattgccatctctgctgtcatcctctgtaaatttatacctgcccttatttgtgcaggctatgcctcatttccttatacaaaattgttaattacaaagcacaccccatactcaacatactgtttgtcttttaagagacgtggttgtgcttaaagaccctccccccattgtaattgtgcgcacttgctccagactttagggtattaactgatttttagagccgggtttaagcctccgttccggtcctcacacagctgccatgtcaataaggactgtttcaagcagaggcattccttctgggagggcactgcagctccttaagacttcatgtgaaacatcataagcactaaaataagacagtgaggggaaattaccagacatggctctgggtttagcagtgccaggcttccagtttagaaagtcattgggcatactatatggcagaggctcttttgaaagcatgcacacctattaaatgcatttttttaaaaaaaaagcctaaggtaatgaaatatcagcatctgcttccaagatctaattgcatggaataagcaagagtttcaaggatgcagaaaacattatgttcagcagagaagcagcttcctgtctacttggaatctcccatgtggcagccctgaggtgggaaccctttatgagtttagaatgtatgttgcagggtgcactgtactgggtgcatcattttaaaaccatattaataataaaagaggaaaccaagacatgcttctccttctcccacttgcatctcaatatgcaaaaggttctgtagtgctgggctctttggctaaaggaatcaaaacatatttgaagaagacaagccagtttaaggagcctacagtttgttcttcaccactcactaagagggcacattgaaatcaaaagcatttttgaagtgtacttagaattctgttttgaacttatctaatcagagataagtatagtcataaagtcactctccttccaccatttggcttcccttatctccacgcattgttcttatctcctgctagtctcagctactgacaatagattatctttgtttagagaggctaattgttttcaaaatccttttaattattgctttggaactaggtgtgcaaatgaggcttaagataattacaacttcacttgtttgtgcagttttactaagcattctccacacataaacaaaacagcagatgatgggacttaaattgcagtgcatctctcattccctggcagcaaaataggccagcattgcaacacacacacacacacacatacacacacacacacacacacacacacctctttatttaatcttcttggagccgctgtcgttctcgctttgagggcagtgtccctctcagagcagggtaaggattatattacaaccggacggttattggagccgggcacagttttatttaatttacaccacatccagcgcctttcgtacgaggttcccaaagcatttttgctaatagcaagtaaagcctctcggcattcctcagtagcagaggtggcactgcaaaacacaaccgcacagatggggaaattgagtccagaagcttcaggaacctgctggtttcaatcgcagaacattaagataccaaatcacaaatgaacgctcggagtctctaggtgctcagcaagtatgctctaaacagctctctgcctggtctcaaatcatggaggtcttttgtgaacgagggaaacgctgtccgactgggagtacttgtgccctttggagggtggtgtgcagcagtggggggaggacagtggtagttggtggggggagggaggaggaaatagggttggactcctcgagctgaagccaacccgaagccactcttccagaggctgcatgcgcggaccctggtctgctcttgaattatttatcgcgttccataagctattcatcaatccattatttattcattccaacattaaagtaattaaatatgagctcaccgggagagggcaagtctgcaagggatgaggacaagagtacggagggagaggggaggaattaaggtttggagtcattgatttgtgcggagttgtgcgcgactccccttaggtgatactggcgacgggtcccgcgttgcagggtcctgacgcgctcacccactccctacctccccacacctccgcagcgcagctccctcagaccccaactggcttcccacctttcctcttccttctcctcccaccactccctacccgcgcagccccggcaacacccccaacccccagctccgcagcctcgccctccccgctctgggcggtgcggcctctctgcccagccgggcggcgcgctggctggccagccccggataggcgccgccggcagccaatcagcgtgccggggacgctgcgcgctttaaggccgctgcggggagaacagaaacc

SEQ ID NO: 24 - DNA, 2049 bp, synthetic construct
  source 1..2049: mol_type = other DNA
ataaatcgtaggaagacttaaagagttaaccgtttgtagatgttcctacttgcacataccacagggctaggtcaaatccaaactgccaaagaaagaaggcaagcaagttctccctgaagggcaggccaccatgtgaataaaacctccagacagatcatgttctaaatgtgccgcagcataccaaaaagtgaggaaaatacggaaacagaatcatgctgtttgacttctgcatcgtgttaactattttttttaaaaaactgctctaactactaaagaaggaaattatgcttagcataaaaatattccagcactgagtgattggtacgttttacttctctcttccagggaaactacttacacttattgattttaaacaccttcacctcagcagcgcatgtaagtgagacactgtaagttaactgtaccttaaagagatcagtatatagatggttctggcgaccaagattacaacaaaagacaaacaacacagaatttttaaagagctgaatagtcagtcaaggatgcatactctcagtctgggctagctcttttttggaaaggttgccaagaatgtccttattccctccagagttatttttcactccactacctctgactaggtgcatcgccagtgcaagaaaatgcgagaagcctctgtttctgttgcaagcaacacctggacctccaaggaaaatgagcactttcttgctaggaacagtctcaaacattttcaagggtgagtctccccttctccaggtttaaccctcttcctccgtagctggagaggctctcctactcgtgcccgccgccttcttcctcccggcgaggattccagggccacaaccagggcagctgcacgccaggccggcctgggtagccgggatgacaccagggtttgccaacgccaaacagggcgcagactccccggcgcgctccggactctccactcgcctaaacctgctctcaagtgaaaaggagtgggaggaggaggattgggagggggcggggggcggaggggatctgttaattcgaggtcctgggggtgtgggagcgggaggcggctccgctgatccctcctccctccccgagtcctgggctccacctcctccgcaggcccccagatatacacacacagccgcggctggacatgcacacccagtgccggctccgggcgcgcacacatgcaggtagccacactcacctgagccggtgcagagggcggcctgggctgggaaggggccccctgcgccgcgggagtggggagagcgggaggtgccacagcgcggacctgcgaactgaggtgatggcccgttttccttaactctcctgcagcacctccgcccctgcgccggctcggctctggccacagcagcgaggacagaggggccccgggcgcggcggactcgcgggcgcacgctcccaggagcgcgccgagaggggcgccgcggcgcaggaaacttgggcgaagttagttgcaagtgccaggcggctgccgagaagagcgaggcggggacgcggccggtgccaggcctgcggcgccgcgtggaggctgcgctgccctccgcggccggcgagctccgggagccctaagtcggcaggcgcgcctgaaggggctcggagcgaacagagccggcatggcccaggggacggccggacgcggctcctcggcgagccgggagaacttcctctgaggccgggcgaccaacgcttgctcaagcctcatccaccctccttcctcgccccacccgtcagtccagcagcgggagaggaagactcgagaggcaggtgctgcgggggacagctcccccctccaaaaaaaaaaaatcacccagccgattgggggtttcccatcggcgcaccctgcccggagccaagaagacaggctggtgctgctgtatttgtatttatatccattgctgcgctctgcgttctcgtggcacgcctggacactcctccgcctccccctcctcttcctcctccagggccacctccccgccttccccacccccatctgcttctgtcaaatgagaaagtcaccgaggagaaccc

SEQ ID NO: 25 - DNA, 1242 bp, synthetic construct
  source 1..1242: mol_type = other DNA
tgcaagaaccctcactggctgaactatcttgccagccccttattttgttttcatattaacctcttttttctagtaaaggagatgtttgctctcaaatttgcataggaatgtaatatttaatttaaaaagatgacccacatatgaccttataaggacagtaaaattaaacaaccggaaagataaagcgggccagttggctcagttctataaaaccagcccacaaggattgtcactattcttaggcttgcgcgggctacatgatgagttccaggactgcctggttacagaccgagactctctcaagagtccagataaacaacaacaaagggggcgaggtggaaatacaggggctgtaagaagtaaatatgatatctgcatgggaggctagccagagaagaaaaaattttcttccgtggttcaatcctccaagggctgaacaggaagttgacgcaggcaggtgaggagcacgagcctagatgggctgcggtgccacccttaatccccacaagcgagttcctccgcaattcgcctgtcccactctcaacttttcttcaactgactctttgctgtggtccctcgctgtggcagtggaaacaactaccactgcgaggtagggaatgtcatgaggggctacctgcagcccttggcttgcagggatgcagggatgcggtcggaacctgaggccccgcccttctcttgccccacgccattaggccacgcccctacccagcactccttcaaccacccccttccccggcgcctcatgaggtcccgcccctctcaaccctagctcttgaggcctccccttcacagccgccccggcgttccttgacttgaggccacgtccctctgctccttcattcccaagaccctacgctttgcgagtcctccctgtcctgctgcctaggaccccgcccctctcagcccttctgccccaagaccccgccccttaggctgttcccgcccactggccaatgaagacccgccctttctttagccgccccgccccggtcccacaaaatcccgcctccggccccgcctcccgcccccttgggcgctccgtagcagtgacgtgcgcaggctgggcactctgcagggctctctggccggcgggtggagaccgatccgggatctgtcccagcaggaagcgtatcccggccgccgtcgtgctgtcgtctccggtgctcgctctcggccgcggtgtcgcgcttgcccttcgcgcccgcagcccggcagcctctc